(19) United States
(12) Patent Application Publication
(10) Pub. No.: US 2002/0004511 A1
(43) Pub. Date: Jan. 10, 2002

(54) Thiophene derivatives useful as anticancer agents

(76) Inventors: Michael Joseph Luzzio, Groton, CT (US); Bingwei Vera Yang, Waterford, CT (US); Matthew Arnold Marx, Waterford, CT (US)

Correspondence Address: Paul H. Ginsburg, Pfizer Inc, 20th Floor, 235 East 42nd Street, New York, NY, 10017-5755, US

(21) Appl. No.: 09/891,087

(22) Filed: Jun. 25, 2001

Related U.S. Application Data
(60) Provisional application No. 60/214,373, filed on Jun. 28, 2000.

(51) Int. Cl. A61K 31/4743; A61K 31/519; C07D 493/04

(52) U.S. Cl. 514/301; 514/258; 514/443; 544/278; 546/114

(57) ABSTRACT

The invention relates to compounds of the formula 1

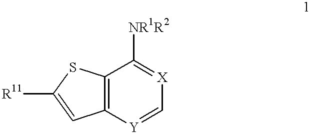

and to pharmaceutically acceptable salts and hydrates thereof, wherein X, Y, R¹, R² and R¹¹ are as defined herein. The invention also relates to pharmaceutical compositions containing the compounds of formula 1 and to methods of treating hyperproliferative disorders in a mammal by administering the compounds of formula 1.

This application claims the benefit of U.S. Provisional Application No. 60/214,373, filed Jun. 28, 2000, which is hereby incorporated in its entirety by reference.

### BACKGROUND OF THE INVENTION

This invention relates to novel thiophene derivatives that are useful in the treatment of hyperproliferative diseases, such as cancers, in mammals. This invention also relates to a method of using such compounds in the treatment of hyperproliferative diseases in mammals, especially humans, and to pharmaceutical compositions containing such compounds.

Compounds that are useful in the treatment of hyperproliferative diseases are also disclosed in the following patent applications: PCT international patent application number PCT/IB97/00675 (filed Jun. 11, 1997), U.S. provisional patent application No. 60/041846 (filed Apr. 9, 1997), U.S. provisional patent application No. 60/031862 (filed Nov. 27, 1996), U.S. provisional patent application No. 60/028881 (filed Oct. 17, 1996), PCT international patent application number PCT/IB97/00584 (filed May 22, 1997), U.S. patent application Ser. No. 08/653,786 (filed May 28, 1996), PCT international patent application publication number WO 96/40142 (published Dec. 19, 1996), PCT international patent application publication number WO 97/13771 (published Apr. 17, 1997), PCT international patent application publication number WO 95/23141 (published Aug. 31, 1995) and United States patent application having attorney reference number PC9882B (filed Feb. 10, 2000). Each of the foregoing United States and PCT international patent applications is incorporated herein by reference in its entirety.

It is known that a cell may become cancerous by virtue of the transformation of a portion of its DNA into an oncogene (i.e. a gene that upon activation leads to the formation of malignant tumor cells). Many oncogenes encode proteins which are aberrant tyrosine kinases capable of causing cell transformation. Alternatively, the overexpression of a normal proto-oncogenic tyrosine kinase may also result in proliferative disorders, sometimes resulting in a malignant phenotype.

Receptor tyrosine kinases are large enzymes that span the cell membrane and possess an extracellular binding domain for growth factors such as epidermal growth factor, a transmembrane domain, and an intracellular portion that functions as a kinase to phosphorylate specific tyrosine residue in proteins and hence to influence cell proliferation. The foregoing tyrosine kinases may be classified as growth factor receptor (e.g. EGFR, PDGFR, FGFR and erbB2) or non-receptor (e.g. c-src and bcr-abl) kinases. It is known that such kinases are often aberrantly expressed in common human cancers such as breast cancer, gastrointestinal cancer such as colon, rectal or stomach cancer, leukemia, and ovarian, bronchial or pancreatic cancer. Aberrant erbB2 activity has been implicated in breast, ovarian, non-small cell lung, pancreatic, gastric and colon cancers. It has also been shown that epidermal growth factor receptor (EGFR) is mutated or overexpressed in many human cancers such as brain, lung, squamous cell, bladder, gastric, breast, head and neck, oesophageal, gynecological and thyroid cancers. Thus, it is believed that inhibitors of receptor tyrosine kinases, such as the compounds of the present invention, are useful as selective inhibitors of the growth of mammalian cancer cells.

It has also been shown that EGFR inhibitors may be useful in the treatment of pancreatitis and kidney disease (such as proliferative glomerulonephritis and diabetes-induced renal disease), and may reduce successful blastocyte implantation and therefore may be useful as a contraceptive. See PCT international application publication number WO 95/19970 (published Jul. 27, 1995).

It is known that polypeptide growth factors such as vascular endothelial growth factor (VEGF) having a high affinity to the human kinase insert-domain-containing receptor (KDR) or the murine fetal liver kinase 1 (FLK-1) receptor have been associated with the proliferation of endothelial cells and more particularly vasculogenesis and angiogenesis. See PCT international application publication number WO 95/21613 (published Aug. 17, 1995). Agents, such as the compounds of the present invention, that are capable of binding to or modulating the KDR/FLK-1 receptor may be used to treat disorders related to vasculogenesis or angiogenesis such as diabetes, diabetic retinopathy, hemangioma, glioma, melanoma, Kaposi's sarcoma and ovarian, breast, lung, pancreatic, prostate, colon and epidermoid cancer.

### SUMMARY OF THE INVENTION

The present invention relates to compounds of the formula 1

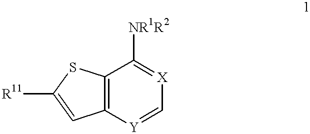

or a pharmaceutically acceptable salt, prodrug or hydrate thereof,

X is N, CH or C—CN;

Y is N, CH, CF, or N→O;

R¹ is H;

R² is 5 to 13 membered heterocyclic, wherein said R² group is optionally substituted by 1 to 5 R⁵ substituents,

each R⁵ is independently selected from halo, cyano, trifluoromethoxy, trifluoromethyl, —C(O)R⁸, —NR⁶C(O)R⁷, —C(O)NR⁶R⁷, —NR⁶R⁷, —(CH₂)ₜNR⁶R⁷, —OR⁹, —SO₂NR⁶R⁷, —NR⁹SO₂NR⁶R⁷, —SO₂R⁶, C₁-C₆ alkyl, C₂-C₆ alkenyl, —(CH₂)ⱼO(CH₂)_{q}NR⁶R⁷, (CH₂)ₜO(CH₂)_{q}OR⁹, —(CH₂)ₜOR⁹, —S(O)ⱼ(C₁-C₆ alkyl), —(CH₂)ₜ(C₆-C₁₀ aryl), —(CH₂)ₜ(5 to 10 membered heterocyclic), —(CH₂)ₜO(CH₂)_{q}(5 to 10 membered heterocyclic), —C(O)(CH₂)ₜ(5 to 10 membered heterocyclic), —(CH₂)ⱼNR⁷(CH₂)_{q}NR⁶R⁷, —(CH₂)ⱼNR⁷CH₂C(O)NR⁶R⁷, —(CH₂)ⱼNR⁹(CH₂)_{q}NR⁹C(O)R⁸, —(CH₂)ⱼNR⁷(CH₂)ₜO(CH₂)_{q}OR⁹, —(CH₂)ⱼNR⁷(CH₂)_{q}S(O)ⱼ(C₁-C₆ alkyl), —(CH₂)ⱼNR⁷(CH₂)ₜR⁶, —SO₂(CH₂)ₜ(C₆-C₁₀ aryl), and —SO₂(CH₂)ₜ(5 to 10 membered heterocyclic), wherein j is an integer from 0 to 2, t is an integer from 0 to 6, q is an integer from 2 to 6, the —(CH₂)_{q}— and —(CH₂)ₜ— moieties of the foregoing R⁵ groups optionally include a carbon-carbon double or triple bond where t is an integer from 2 to 6, and the alkyl, aryl and heterocyclic moieties of the foregoing R⁵ groups are optionally substituted by 1 to 3 substituents independently selected from halo, cyano, trifluoromethyl, —C(O)R⁸, —(CH₂)ₜOR⁹, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6;

each R⁶ and R⁷ is independently selected from H, C₁-C₆ alkyl, —(CH₂)ₜ(C₆-C₁₀ aryl), —(CH₂)ₜ(C₆-C₁₀ cycloalkyl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, and the alkyl, aryl and heterocyclic moieties of the foregoing R⁶ and R⁷ groups are optionally substituted by 1 to 3 substituents independently selected from halo, cyano, trifluoromethyl, —C(O)R⁸, —NR⁹C(O)R¹⁰, —C(O)NR⁹R¹⁰, —NR⁹R¹⁰, C₁-C₆ alkyl, —(CH₂)ₜ(C₆-C₁₀ aryl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, with the proviso that where R⁶ and R⁷ are both attached to the same nitrogen, then R⁶ and R⁷ are not both bonded to the nitrogen directly through an oxygen;

each R⁸ is independently selected from H, C₁-C₁₀ alkyl, —O(C₁-C₁₀ alkyl), —(CH₂)ₜ(C₆-C₁₀ aryl), and —(CH₂)ₜ(5 to 10 membered heterocyclic), wherein t is an integer from 0 to 6;

each R⁹ and R¹⁰ is independently selected from H and C₁-C₆ alkyl; and,

R¹¹ is selected from the group consisting of imidazolyl, oxazolyl, oxadiazolyl, isoxazolyl, thiazolyl and thiadiazolyl, wherein said imidazolyl, oxazolyl, oxadiazolyl, isoxazolyl, thiazolyl and thiadiazolyl are optionally substituted by 1 to 5 R⁵ groups with the proviso that compound 1 is not

[2-(3-Methyl-3H-imidazol-4-yl)-thieno[3,2-b]pyridin-7-yl]-(2-methyl-1H-indol-5-yl)-amine;

2-{1-Methyl-5-[7-(2-methyl-1H-indol-5-ylamino)-thieno[3,2-b]pyridin-2-yl]-1H-imidazol-2-yl}-propan-2-ol;

[2-(1-Methyl-1H-imidazol-2-yl)-thieno[3,2-b]pyridin-7-yl]-(2-methyl-1H-indol-5-yl)-amine;

(2-Methyl-1H-indol-5-yl)-(2-thiazol-2-yl-thieno[3,2-b]pyridin-7-yl)-amine; or

2-{2-[7-(2-Methyl-1H-indol-5-ylamino)-thieno[3,2-b]pyridin-2-yl]-thiazol-5-yl}-propan-2-ol.

Preferred compounds include those of formula 1, wherein X is CH.

Other preferred compounds include those of formula 1, wherein R¹¹ is imidazolyl, oxazolyl, or thiazolyl, wherein said imidazolyl, oxazolyl, and thiazolyl are optionally substituted by 1 to 5 R⁵ groups.

In one preferred embodiment, compounds include those of formula 1 wherein each R⁵ when present is independently selected from cyano, —C(O)R⁸, —NR⁶C(O)R⁷, —C(O)NR⁶R⁷, —NR⁶R⁷, —OR⁹, C₁-C₆ alkyl, —(CH₂)ⱼO(CH₂)_{q}NR⁶R⁷, —(CH₂)ₜO(CH₂)_{q}OR⁹, —(CH₂)ₜOR⁹, —(CH₂)ₜ(5 to 10 membered heterocyclic), —C(O)(CH₂)ₜ(5 to 10 membered heterocyclic), —(CH₂)ⱼNR⁷(CH₂)_{q}NR⁶R⁷, —(CH₂)ⱼNR⁷CH₂C(O)NR⁶R⁷, —(CH₂)ⱼNR⁷(CH₂)_{q}NR⁹C(O)R⁸, —(CH₂)ⱼNR⁷(CH₂)ₜO(CH₂)_{q}OR⁹, and —(CH₂)ⱼNR⁷(CH₂)ₜR⁶, wherein j is an integer from 0 to 2, t is an integer from 0 to 6, q is an integer from 2 to 6, the —(CH₂)_{q}— and —(CH₂)ₜ— moieties of the foregoing R⁵ groups optionally include a carbon-carbon double or triple bond where t is an integer from 2 to 6, and the alkyl, aryl and heterocyclic moieties of the foregoing R⁵ groups are optionally substituted by 1 to 3 substituents independently selected from halo, cyano, trifluoromethyl, —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

In the most preferred embodiment, compounds include those of formula 1, wherein each R⁵ when present is independently selected from —C(O)R⁸, —C(O)NR⁶R⁷, —NR⁶R⁷, —OR⁹, C₁-C₆ alkyl, —C(O)(CH₂)ₜ(5 to 10 membered heterocyclic), wherein t is an integer from 0 to 6, the —(CH₂)ₜ— moiety of the foregoing R⁵ group optionally includes a carbon-carbon double or triple bond when t is an integer from 2 to 6, and the alkyl and heterocyclic moieties of the foregoing R⁵ groups are optionally substituted by 1 to 3 substituents independently selected from halo, cyano, trifluoromethyl, —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

In a most preferred embodiment, compounds include those of formula 1, wherein each R⁵ when present is independently selected from —C(O)R⁸, —C(O)NR⁶R⁷, C₁-C₆ alkyl, —C(O)(CH₂)ₜ(5 to 10 membered heterocyclic), wherein t is an integer from 0 to 6, the —(CH₂)ₜ— moiety of the foregoing R⁵ group optionally includes a carbon-carbon double or triple bond when t is an integer from 2 to 6, and the alkyl and heterocyclic moieties of the foregoing R⁵ groups are optionally substituted by 1 to 3 substituents independently selected from —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

Other preferred compounds include those of formula 1, wherein R² is a group of the formula

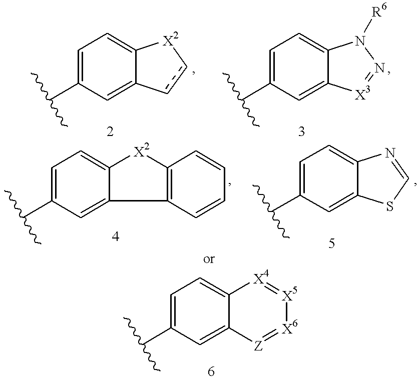

wherein X² is —S—, —N(R⁶)— or O, and X³, X⁴, X⁵, X⁶, and Z is N or CH, the dashed line in formula 2 represents an optional double bond, and the above R² groups of formulas 2, 4 and 6 are optionally substituted by 1 to 5 R⁵ substituents and the R² groups of formulas 3 and 5 are optionally substituted by 1 to 3 R⁵ substituents.

Specifically preferred compounds include those wherein R² group is a group of formula 2, wherein said group is optionally substituted by 1 to 3 R⁵ substituents.

Specific embodiments of the present invention include the following compounds:

{1-Methyl-5-[7-(2-methyl-1H-indol-5-ylamino)-thieno[3,2-b]pyridin-2-yl]-1H-imidazol-2-yl}-morpholin-4-yl-methanone;

{1-Methyl-5-[7-(2-methyl-1H-indol-5-ylamino)-thieno[3,2-b]pyridin-2-yl]-1H-imidazol-2-yl}-(4-methyl-piperazin-1-yl)-methanone;

1-Methyl-5-[7-(2-methyl-1H-indol-5-ylamino)-thieno[3,2-b]pyridin-2-yl]-1H-imidazole-2-carboxylic acid dimethylamide;

1-Methyl-5-[7-(2-methyl-1H-indol-5-ylamino)-thieno[3,2-b]pyridin-2-yl]-1H-imidazole-2-carboxylic acid methylamide;

2-{1-Methyl-5-[7-(2-methyl-1H-indol-5-ylamino)-thieno[3,2-b]pyridin-2-yl]-1H-imidazol-2-yl}-propane-1,2-diol;

1-Methyl-5-[7-(2-methyl-1H-indol-5-ylamino)-thieno[3,2-b]pyridin-2-yl]-1H-imidazole-2-carboxylic acid amide;

2-{2-[7-(2-Methyl-1H-indol-5-ylamino)-thieno[3,2-b]pyridin-2-yl]-thiazol-4-yl}-propan-2-ol; pharmaceutically acceptable salts of said compounds; solvates of said compounds; and prodrugs of said compounds.

The present invention also relates to a compound of the formula 1

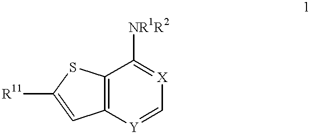

or a pharmaceutically acceptable salt, prodrug or hydrate thereof, wherein X, Y, R¹, R², R⁵, R⁶, R⁷, R⁸, R⁹, R¹⁰, and R¹¹ are as defined above, with the proviso that compound 1 is not

[2-(3-Methyl-3H-imidazol-4-yl)-thieno[3,2-b]pyridin-7-yl]-(2-methyl-1H-indol-5-yl)-amine;

2-{1-Methyl-5-[7-(2-methyl-1H-indol-5-ylamino)-thieno[3,2-b]pyridin-2-yl]-1H-imidazol-2-yl}-propan-2-ol;

[2-(1-Methyl-1H-imidazol-2-yl)-thieno[3,2-b]pyridin-7-yl]-(2-methyl-1H-indol-5-yl)-amine;

(2-Methyl-1H-indol-5-yl)-(2-thiazol-2-yl-thieno[3,2-b]pyridin-7-yl)-amine;

2-{2-[7-(2-Methyl-1H-indol-5-ylamino)-thieno[3,2-b]pyridin-2-yl]-thiazol-5-yl}-propan-2-ol;

[2-(2-Ethoxy-thiazol-5-yl)-thieno[3,2-b]pyridin-7-yl]-(2-methyl-1H-indol-5-yl)-amine;

(2-Methyl-1H-indol-5-yl)-[2-(4-methyl-thiazol-2-yl)-thieno[3,2-b]pyridin-7-yl]-amine;

[2-(3-Methoxymethyl-3H-imidazol-4-yl)-thieno[3,2-b]pyridin-7-yl]-(2-methyl-1H-indol-5-yl)-amine;

{2-[5-(4-Methoxy-phenyl)-oxazol-2-yl]-thieno[3,2-b]pyridin-7-yl}-(2-methyl-1H-indol-5-yl)-amine; or

2-{4-Methyl-2-[7-(2-methyl-1H-indol-5-ylamino)-thieno[3,2-b]pyridin-2-yl]-thiazol-5-yl}-propan-2-ol.

In one preferred embodiment of the present invention is directed to a compound having the formula 1

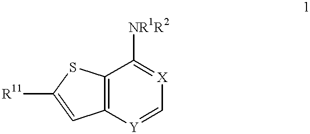

or a pharmaceutically acceptable salt, prodrug or hydrate thereof, wherein X is CH; Y is N; R¹ is H;

R² is 5 to 13 membered heterocyclic, wherein said R² group is optionally substituted by 1 to 5 R⁵ substituents;

each R⁵ is independently selected from cyano, —C(O)R⁸, —NR⁶C(O)R⁷, —C(O)NR⁶R⁷, —NR⁶R⁷, —OR⁹, —NR⁹SO₂NR⁶R⁷, —SO₂R⁶, C₁-C₆ alkyl, —(CH₂)ⱼO(CH₂)_{q}NR⁶R⁷, —(CH₂)ₜO(CH₂)_{q}OR⁹, —(CH₂)ₜOR⁹, —(CH₂)ₜ(5 to 10 membered heterocyclic), —C(O)(CH₂)ₜ(5 to 10 membered heterocyclic), —(CH₂)ⱼNR⁷(CH₂)_{q}NR⁶R⁷, —(CH₂)ⱼNR⁷CH₂C(O)NR⁶R⁷, —(CH₂)ⱼNR⁷(CH₂)_{q}NR⁹C(O)R⁸, —(CH₂)ⱼNR⁷(CH₂)ₜO(CH₂)_{q}OR⁹, and —(CH₂)ⱼNR⁷(CH₂)ₜR⁶, wherein j is an integer from 0 to 2, t is an integer from 0 to 6, q is an integer from 2 to 6, the —(CH₂)_{q}— and —(CH₂)ₜ— moieties of the foregoing R⁵ groups optionally include a carbon-carbon double or triple bond where t is an integer from 2 to 6, and the alkyl, aryl and heterocyclic moieties of the foregoing R⁵ groups are optionally substituted by 1 to 3 substituents independently selected from halo, cyano, trifluoromethyl, —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6; R⁶, R⁷, R⁸, R⁹, R¹⁰, and R¹¹ are as defined above, with the proviso that compound 1 is not

[2-(3-Methyl-3H-imidazol-4-yl)-thieno[3,2-b]pyridin-7-yl]-(2-methyl-1H-indol-5-yl)-amine;

2-{1-Methyl-5-[7-(2-methyl-1H-indol-5-ylamino)-thieno[3,2-b]pyridin-2-yl]-1H-imidazol-2-yl}-propan-2-ol;

[2-(1-Methyl-1H-imidazol-2-yl)-thieno[3,2-b]pyridin-7-yl]-(2-methyl-1H-indol-5-yl)-amine;

(2-Methyl-1H-indol-5-yl)-(2-thiazol-2-yl-thieno[3,2-b]pyridin-7-yl)-amine; or

2-{2-[7-(2-Methyl-1H-indol-5-ylamino)-thieno[3,2-b]pyridin-2-yl]-thiazol-5-yl}-propan-2-ol.

In one preferred embodiment of the present invention is directed to a compound having the formula 1

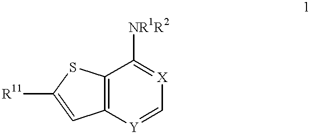

or a pharmaceutically acceptable salt, prodrug or hydrate thereof, wherein X is CH; Y is N; R¹ is H;

R² is 5 to 13 membered heterocyclic, wherein said R² group is optionally substituted by 1 to 5 R⁵ substituents,

each R⁵ is independently selected from cyano, —C(O)R⁸, —NR⁶C(O)R⁷, —C(O)NR⁶R⁷, —NR⁶R⁷, —OR⁹, —NR⁹SO₂NR⁶R⁷, —SO₂R⁶, C₁-C₆ alkyl, —(CH₂)ⱼO(CH₂)_{q}NR⁶R⁷, —(CH₂)ₜO(CH₂)_{q}OR⁹, —(CH₂)ₜOR⁹, —(CH₂)ₜ(5 to 10 membered heterocyclic), —C(O)(CH₂)ₜ(5 to 10 membered heterocyclic), —(CH₂)ⱼNR⁷(CH₂)_{q}NR⁶R⁷, —(CH₂)ⱼNR⁷CH₂C(O)NR⁶R⁷, —(CH₂)ⱼNR⁷(CH₂)_{q}NR⁹C(O)R⁸, —(CH₂)ⱼNR⁷(CH₂)ₜO(CH₂)_{q}OR⁹, and —(CH₂)ⱼNR⁷(CH₂)ₜR⁶, wherein j is an integer from 0 to 2, t is an integer from 0 to 6, q is an integer from 2 to 6, the —(CH₂)_{q}— and —(CH₂)ₜ— moieties of the foregoing R⁵ groups optionally include a carbon-carbon double or triple bond where t is an integer from 2 to 6, and the alkyl, aryl and heterocyclic moieties of the foregoing R⁵ groups are optionally substituted by 1 to 3 substituents independently selected from halo, cyano, trifluoromethyl, —C(O)R⁸, —NR⁶C(O)R⁷, —C(O)NR⁶R⁷, (CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6; R⁶, R⁷, R⁸, R⁹, R¹⁰, and R¹¹ are as defined above, with the proviso that compound 1 is not

[2-(3-Methyl-3H-imidazol-4-yl)-thieno[3,2-b]pyridin-7-yl]-(2-methyl-1H-indol-5-yl)-amine;

2-{1-Methyl-5-[7-(2-methyl-1H-indol-5-ylamino)-thieno[3,2-b]pyridin-2-yl]-1H-imidazol-2-yl}-propan-2-ol;

[2-(1-Methyl-1H-imidazol-2-yl)-thieno[3,2-b]pyridin-7-yl]-(2-methyl-1H-indol-5-yl)-amine;

(2-Methyl-1H-indol-5-yl)-(2-thiazol-2-yl-thieno[3,2-b]pyridin-7-yl)-amine;

2-{2-[7-(2-Methyl-1H-indol-5-ylamino)-thieno[3,2-b]pyridin-2-yl]-thiazol-5-yl}-propan-2-ol;

[2-(2-Ethoxy-thiazol-5-yl)-thieno[3,2-b]pyridin-7-yl]-(2-methyl-1H-indol-5-yl)-amine;

(2-Methyl-1H-indol-5-yl)-[2-(4-methyl-thiazol-2-yl)-thieno[3,2-b]pyridin-7-yl]-amine;

[2-(3-Methoxymethyl-3H-imidazol-4-yl)-thieno[3,2-b]pyridin-7-yl]-(2-methyl-1H-indol-5-yl)-amine;

{2-[5-(4-Methoxy-phenyl)-oxazol-2-yl]-thieno[3,2-b]pyridin-7-yl-(2-methyl-1H-indol-5-yl)-amine; or

2-{4-Methyl-2-[7-(2-methyl-1H-indol-5-ylamino)-thieno[3,2-b]pyridin-2-yl]-thiazol-5-yl}-propan-2-ol.

One embodiment of the present invention is directed to a compound having the formula 1

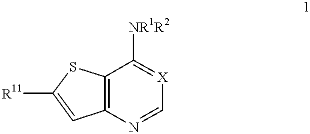

or a pharmaceutically acceptable salt, prodrug or hydrate thereof,

wherein X is CH;

R¹ is H;

R² is

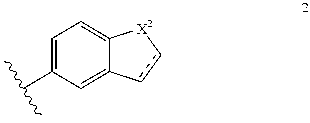

X² is —N(R⁶)—, the dashed line in formula 2 represents an optional double bond, and the above R² group of formula 2 is optionally substituted by 1 to 5 R⁵ substituents;

each R⁵ is independently selected from —C(O)R⁸, —C(O)NR⁶R⁷, —NR⁶R⁷, —OR⁹, C₁-C₆ alkyl, —C(O)(CH₂)ₜ(5 to 10 membered heterocyclic), wherein t is an integer from 0 to 6, the —(CH₂)ₜ— moiety of the foregoing R⁵ group optionally includes a carbon-carbon double or triple bond when t is an integer from 2 to 6, and the alkyl and heterocyclic moieties of the foregoing R⁵ groups are optionally substituted by 1 to 3 substituents independently selected from halo, cyano, trifluoromethyl, —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6;

each R⁶ and R⁷ is independently selected from H, C₁-C₆ alkyl, —(CH₂)ₜ(C₆-C₁₀ aryl), —(CH₂)ₜ(C₆-C₁₀ cycloalkyl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, and the alkyl, aryl and heterocyclic moieties of the foregoing R⁶ and R⁷ groups are optionally substituted by 1 to 3 substituents independently selected from halo, cyano, trifluoromethyl, —C(O)R⁸, —NR⁹C(O)R¹⁰, —C(O)NR⁹R¹⁰, —NR⁹R¹⁰, C₁-C₆ alkyl, —(CH₂)ₜ(C₆-C₁₀ aryl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, with the proviso that where R⁶ and R⁷ are both attached to the same nitrogen, then R⁶ and R⁷ are not both bonded to the nitrogen directly through an oxygen;

each R⁸ is independently selected from H, C₁-C₁₀ alkyl, —O(C₁-C₁₀ alkyl), —(CH₂)ₜ(C₆-C₁₀ aryl), and —(CH₂)ₜ(5 to 10 membered heterocyclic), wherein t is an integer from 0 to 6;

each R⁹ and R¹⁰ is independently selected from H and C₁-C₆ alkyl; and,

R¹¹ is selected from the group consisting of imidazolyl, oxazolyl, or thiazolyl, wherein said imidazolyl, oxazolyl, or thiazolyl are optionally substituted by 1 to 5 R⁵ groups with the proviso that compound 1 is not

[2-(3-Methyl-3H-imidazol4-yl)-thieno[3,2-b]pyridin-7-yl]-(2-methyl-1H-indol-5-yl)-amine;

2-{1-Methyl-5-[7-(2-methyl-1H-indol-5-ylamino)-thieno[3,2-b]pyridin-2-yl]-1H-imidazol-2-yl}-propan-2-ol;

[2-(1-Methyl-1H-imidazol-2-yl)-thieno[3,2-b]pyridin-7-yl]-(2-methyl-1H-indol-5-yl)-amine;

(2-Methyl-1H-indol-5-yl)-(2-thiazol-2-yl-thieno[3,2-b]pyridin-7-yl)-amine; or

2-{2-[7-(2-Methyl-1H-indol-5-ylamino)-thieno[3,2-b]pyridin-2-yl]-thiazol-5-yl}-propan-2-ol.

Another embodiment of the invention is directed to a compound having the formula 1

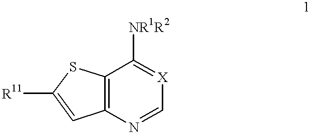

or a pharmaceutically acceptable salt, prodrug or hydrate thereof,

wherein X is CH;

R¹ is H;

R² is

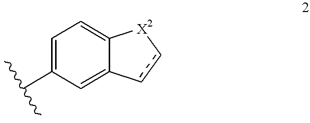

X² is —N(R⁶)—, the dashed line in formula 2 represents an optional double bond, and the above R² group of formula 2 is optionally substituted by 1 to 5 R⁵ substituents;

each R⁵ is independently selected from —C(O)R⁸, —C(O)NR⁶R⁷, —NR⁶R⁷, —OR⁹, C₁-C₆ alkyl, —C(O)(CH₂)ₜ(5 to 10 membered heterocyclic), wherein t is an integer from 0 to 6, the —(CH₂)ₜ— moiety of the foregoing R⁵ group optionally includes a carbon-carbon double or triple bond when t is an integer from 2 to 6, and the alkyl and heterocyclic moieties of the foregoing R⁵ groups are optionally substituted by 1 to 3 substituents independently selected from halo, cyano, trifluoromethyl, —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6;

each R⁶ and R⁷ is independently selected from H, C₁-C₆ alkyl, —(CH₂)ₜ(C₆-C₁₀ aryl), —(CH₂)ₜ(C₆-C₁₀ cycloalkyl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, and the alkyl, aryl and heterocyclic moieties of the foregoing R⁶ and R⁷ groups are optionally substituted by 1 to 3 substituents independently selected from halo, cyano, trifluoromethyl, —C(O)R⁸, —NR⁹C(O)R¹⁰, —C(O)NR⁹R¹⁰, —NR⁹R¹⁰, C₁-C₆ alkyl, —(CH₂)ₜ(C₆-C₁₀ aryl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, with the proviso that where R⁶ and R⁷ are both attached to the same nitrogen, then R⁶ and R⁷ are not both bonded to the nitrogen directly through an oxygen;

each R⁸ is independently selected from H, C₁-C₁₀ alkyl, —O(C₁-C₁₀ alkyl), —(CH₂)ₜ(C₆-C₁₀ aryl), and —(CH₂)ₜ(5 to 10 membered heterocyclic), wherein t is an integer from 0 to 6;

each R⁹ and R¹⁰ is independently selected from H and C₁-C₆ alkyl; and,

R¹¹ is selected from the group consisting of imidazolyl, oxazolyl, or thiazolyl, wherein said imidazolyl, oxazolyl, or thiazolyl are optionally substituted by 1 to 5 R⁵ groups with the proviso that compound 1 is not

[2-(3-Methyl-3H-imidazol-4-yl)-thieno[3,2-b]pyridin-7-yl]-(2-methyl-1H-indol-5-yl)-amine;

2-{1-Methyl-5-[7-(2-methyl-1H-indol-5-ylamino)-thieno[3,2-b]pyridin-2-yl]-1H-imidazol-2-yl}-propan-2-ol;

[2-(1-Methyl-1H-imidazol-2-yl)-thieno[3,2-b]pyridin-7-yl]-(2-methyl-1H-indol-5-yl)-amine;

(2-Methyl-1H-indol-5-yl)-(2-thiazol-2-yl-thieno[3,2-b]pyridin-7-yl)-amine;

2-{2-[7-(2-Methyl-1H-indol-5-ylamino)-thieno[3,2-b]pyridin-2-yl]-thiazol-5-yl}-propan-2-ol;

[2-(2-Ethoxy-thiazol-5-yl)-thieno[3,2-b]pyridin-7-yl]-(2-methyl-1H-indol-5-yl)-amine;

(2-Methyl-1H-indol-5-yl)-[2-(4-methyl-thiazol-2-yl)-thieno[3,2-b]pyridin-7-yl]-amine;

[2-(3-Methoxymethyl-3H-imidazol-4-yl)-thieno[3,2-b]pyridin-7-yl]-(2-methyl-1H-indol-5-yl)-amine;

{2-[5-(4-Methoxy-phenyl)-oxazol-2-yl]-thieno[3,2-b]pyridin-7-yl}-(2-methyl-1H-indol-5-yl)-amine; or

2-{4-Methyl-2-[7-(2-methyl-1H-indol-5-ylamino)-thieno[3,2-b]pyridin-2-yl]-thiazol-5-yl}-propan-2-ol.

The invention also relates to a compound having the formula 1

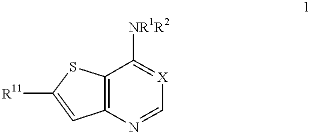

or a pharmaceutically acceptable salt, prodrug or hydrate thereof,

wherein X is CH;

R¹ is H;

R² is

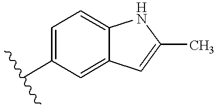

wherein each R⁵ is independently selected from —C(O)R⁸, —C(O)NR⁶R⁷, C₁-C₆ alkyl, —C(O)(CH₂)ₜ(5 to 10 membered heterocyclic), wherein t is an integer from 0 to 6;

each R⁶ and R⁷ is independently selected from H, C₁-C₆ alkyl, —(CH₂)ₜ(C₆-C₁₀ aryl), —(CH₂)ₜ(C₆-C₁₀ cycloalkyl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, and the alkyl, aryl and heterocyclic moieties of the foregoing R⁶ and R⁷ groups are optionally substituted by 1 to 3 substituents independently selected from halo, cyano, trifluoromethyl, —C(O)R⁸, —NR⁹C(O)R¹⁰, —C(O)NR⁹R¹⁰, —NR⁹R¹⁰, C₁-C₆ alkyl, —(CH₂)ₜ(C₆-C₁₀ aryl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, with the proviso that where R⁶ and R⁷ are both attached to the same nitrogen, then R⁶ and R⁷ are not both bonded to the nitrogen directly through an oxygen;

each R⁸ is independently selected from H, C₁-C₁₀ alkyl, —(CH₂)ₜ(C₆-C₁₀ aryl), and —(CH₂)ₜ(⁵ to 10 membered heterocyclic), wherein t is an integer from 0 to 6;

each R⁹ and R¹⁰ is independently selected from H and C₁-C₆ alkyl; and,

R¹¹ is imidazolyl, thiazolyl or oxazolyl, wherein said imidazolyl, thiazolyl or oxazolyl are optionally substituted by 1 to 5 R⁵ groups with the proviso that compound 1 is not

[2-(3-Methyl-3H-imidazol-4-yl)-thieno[3,2-b]pyridin-7-yl]-(2-methyl-1H-indol-5-yl)-amine;

2-{1-Methyl-5-[7-(2-methyl-1H-indol-5-ylamino)-thieno[3,2-b]pyridin-2-yl]-1H-imidazol-2-yl}-propan-2-ol;

[2-(1-Methyl-1H-imidazol-2-yl)-thieno[3,2-b]pyridin-7-yl]-(2-methyl-1H-indol-5-yl)-amine;

(2-Methyl-1H-indol-5-yl)-(2-thiazol-2-yl-thieno[3,2-b]pyridin-7-yl)-amine; or

2-{2-[7-(2-Methyl-1H-indol-5-ylamino)-thieno[3,2-b]pyridin-2-yl]-thiazol-5-yl}-propan-2-ol.

The invention further relates to a compound having the formula 1

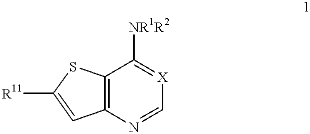

or a pharmaceutically acceptable salt, prodrug or hydrate thereof,

wherein X is CH;

R¹ is H

R² is

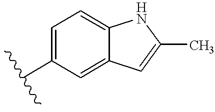

wherein each R⁵ is independently selected from —C(O)R⁸, —C(O)NR⁶R⁷, C₁-C₆ alkyl, —C(O)(CH₂)ₜ(5 to 10 membered heterocyclic), wherein t is an integer from 0 to 6;

wherein each R⁶ and R⁷ is independently selected from H, C₁-C₆ alkyl, —(CH₂)ₜ(5 to 10 membered heterocyclic), and —(CH₂)ₜOR⁹, wherein t is an integer from 0 to 6, with the proviso that where R⁶ and R⁷ are both attached to the same nitrogen, then R⁶ and R ⁷ are not both bonded to the nitrogen directly through an oxygen;

each R⁸ is independently selected from H, C₁-C₁₀ alkyl, —(CH₂)ₜ(C₆-C₁₀ aryl), and —(CH₂)ₜ(5 to 1 0 membered heterocyclic), wherein t is an integer from 0 to 6;

each R⁹ and R¹⁰ is independently selected from H and C₁-C₆ alkyl; and,

R¹¹ is thiazolyl wherein said thiazolyl is optionally substituted by 1 to 5 R⁵ groups with the proviso that compound 1 is not

(2-Methyl-1H-indol-5-yl)-(2-thiazol-2-yl-thieno[3,2-b]pyridin-7-yl)-amine; or

2-{2-[7-(2-Methyl-1H-indol-5-ylamino)-thieno[3,2-b]pyridin-2-yl]-thiazol-5-yl}-propan-2-ol.

Another embodiment of the invention relates to a compound having the formula 1

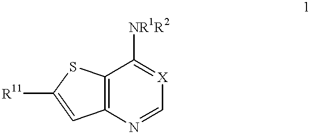

or a pharmaceutically acceptable salt, prodrug or hydrate thereof,

wherein X is CH;

R¹ is H;

R² is

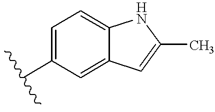

wherein each R⁵ is independently selected from —C(O)R⁸, —C(O)NR⁶R⁷, C₁-C₆ alkyl, —C(O)(CH₂)ₜ(5 to 10 membered heterocyclic), wherein t is an integer from 0 to 6;

wherein each R⁶ and R⁷ is independently selected from H, C₁-C₆ alkyl, —(CH₂)ₜ(5 to 10 membered heterocyclic), and —(CH₂)ₜOR⁹, wherein t is an integer from 0 to 6, with the proviso that where R⁶ and R⁷ are both attached to the same nitrogen, then R⁶ and R⁷ are not both bonded to the nitrogen directly through an oxygen;

each R⁸ is independently selected from H, C₁-C₁₀ alkyl, —(CH₂)ₜ(C₆-C₁₀ aryl), and —(CH₂)ₜ(5 to 10 membered heterocyclic), wherein t is an integer from 0 to 6;

each R⁹ and R¹⁰ is independently selected from H and C₁-C₆ alkyl; and,

R¹¹ is imidazolyl wherein said imidazolyl is optionally substituted by 1 to 5 R⁵ groups with the proviso that compound 1 is not

[2-(3-Methyl-3H-imidazol-4-yl)-thieno[3,2-b]pyridin-7-yl]-(2-methyl-1H-indol-5-yl)-amine;

2-{1-Methyl-5-[7-(2-methyl-1H-indol-5-ylamino)-thieno[3,2-b]pyridin-2-yl]-1H-imidazol-2-yl}-propan-2-ol; or

[2-(1-Methyl-1H-imidazol-2-yl)-thieno[3,2-b]pyridin-7-yl]-(2-methyl-1H-indol-5-yl)-amine.

Another embodiment of the invention relates to a compound having the formula 1

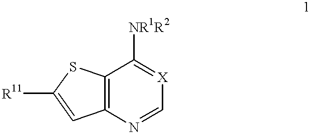

or a pharmaceutically acceptable salt, prodrug or hydrate thereof,

wherein X is CH;

R¹ is H;

R² is

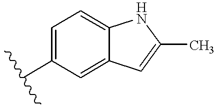

wherein each R⁵ is independently selected from —C(O)R⁸, —C(O)NR⁶R⁷, C₁-C₆ alkyl, —C(O)(CH₂)ₜ(5 to 10 membered heterocyclic), wherein t is an integer from 0 to 6;

wherein each R⁶ and R⁷ is independently selected from H, C₁-C₆ alkyl, —(CH₂)ₜ(5 to 10 membered heterocyclic), and —(CH₂)ₜOR⁹, wherein t is an integer from 0 to 6, with the proviso that where R⁶ and R⁷ are both attached to the same nitrogen, then R⁶ and R⁷ are not both bonded to the nitrogen directly through an oxygen;

each R⁸ is independently selected from H, C₁-C₁₀ alkyl, —(CH₂)ₜ(C₆-C₁₀ aryl), and —(CH₂)ₜ(5 to 10 membered heterocyclic), wherein t is an integer from 0 to 6;

each R⁹ and R¹⁰ is independently selected from H and C₁-C₆ alkyl; and,

R¹¹ is oxazolyl wherein said oxazolyl is optionally substituted by 1 to 5 R⁵ groups.

One embodiment of the present invention is directed to a compound of formula 1

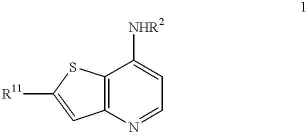

or a pharmaceutically acceptable salt, prodrug or hydrate thereof,

R² is

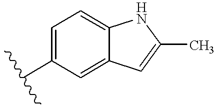

wherein each R⁵ is independently selected from —C(O)R⁸, —C(O)NR⁶R⁷, C₁-C₆ alkyl, —C(O)(CH₂)ₜ(5 to 10 membered heterocyclic), wherein t is an integer from 0 to 6;

wherein each R⁶ and R⁷ is independently selected from H, C₁-C₆ alkyl, —(CH₂)ₜ(5 to 10 membered heterocyclic), and —(CH₂)ₜOR⁹, wherein t is an integer from 0 to 6 or R⁶ and R⁷ when both are attached to the same nitrogen may be taken together to form a 5 to 10 membered heterocyclic, with the proviso that where R⁶ and R⁷ are both attached to the same nitrogen, then R⁶ and R⁷ are not both bonded to the nitrogen directly through an oxygen;

each R⁸ is independently selected from H, C₁-C₁₀ alkyl, —(CH₂)ₜ(C₆-C₁₀ aryl), and —(CH₂)ₜ(5 to 10 membered heterocyclic), wherein t is an integer from 0 to 6;

each R⁹ and R¹⁰ is independently selected from H and C₁-C₆ alkyl; and,

R¹¹ is selected from the group consisting imidazolyl, oxazolyl, or thiazolyl and wherein said imidazolyl, oxazolyl, and thiazolyl are optionally substituted by 1 to 5 R⁵ groups with the proviso that compound 1 is not

[2-(3-Methyl-3H-imidazol-4-yl)-thieno[3,2-b]pyridin-7-yl]-(2-methyl-1H-indol-5-yl)-amine;

2-{1-Methyl-5-[7-(2-methyl-1H-indol-5-ylamino)-thieno[3,2-b]pyridin-2-yl]-1H-imidazol-2-yl}-propan-2-ol;

[2-(1-Methyl-1H-imidazol-2-yl)-thieno[3,2-b]pyridin-7-yl]-(2-methyl-1H-indol-5-yl)-amine;

[2-(2-Ethoxy-thiazol-5-yl)-thieno[3,2-b]pyridin-7-yl]-(2-methyl-1H-indol-5-yl)-amine;

(2-Methyl-1H-indol-5-yl)-[2-(4-methyl-thiazol-2-yl)-thieno[3,2-b]pyridin-7-yl]-amine;

[2-(3-Methoxymethyl-3H-imidazol-4-yl)-thieno[3,2-b]pyridin-7-yl]-(2-methyl-1H-indol-5-yl)-amine;

{2-[5-(4-Methoxy-phenyl)-oxazol-2-yl]-thieno[3,2-b]pyridin-7-yl}-(2-methyl-1H-indol-5-yl)-amine; or

2-{4-Methyl-2-[7-(2-methyl-1H-indol-5-ylamino)-thieno[3,2-b]pyridin-2-yl]-thiazol-5-yl}-propan-2-ol.

Preferred compounds include those of formula 1, wherein R² is 2-methyl-1H-indol-5-ylamino.

Preferred compounds include those of formula 1, wherein R¹¹ is thiazolyl and said thiazolyl is optionally substituted by 1 to 5 R⁵ groups.

The invention also relates to a pharmaceutical composition for the treatment of a hyperproliferative disorder in a mammal which comprises a therapeutically effective amount of a compound of formula 1, or a pharmaceutically acceptable salt, prodrug or hydrate thereof, and a pharmaceutically acceptable carrier. In one embodiment, said pharmaceutical composition is for the treatment of cancer such as brain, lung, squamous cell, bladder, gastric, pancreatic, breast, head, neck, renal, kidney, ovarian, prostate, colorectal, oesophageal, gynecological or thyroid cancer. In another embodiment, said pharmaceutical composition is for the treatment of a non-cancerous hyperproliferative disorder such as benign hyperplasia of the skin (e.g., psoriasis) or prostate (e.g., benign prostatic hypertropy (BPH)).

The invention also relates to a pharmaceutical composition for the treatment of pancreatitis or kidney disease (including proliferative glomerulonephritis and diabetes-induced renal disease) in a mammal which comprises a therapeutically effective amount of a compound of formula 1, or a pharmaceutically acceptable salt, prodrug or hydrate thereof, and a pharmaceutically acceptable carrier.

The invention also relates to a pharmaceutical composition for the prevention of blastocyte implantation in a mammal which comprises a therapeutically effective amount of a compound of formula 1, or a pharmaceutically acceptable salt, prodrug or hydrate thereof, and a pharmaceutically acceptable carrier.

The invention also relates to a pharmaceutical composition for treating a disease related to vasculogenesis or angiogenesis in a mammal which comprises a therapeutically effective amount of a compound of formula 1, or a pharmaceutically acceptable salt, prodrug or hydrate thereof, and a pharmaceutically acceptable carrier. In one embodiment, said pharmaceutical composition is for treating a disease selected from the group consisting of tumor angiogenesis, chronic inflammatory disease such as rheumatoid arthritis, atherosclerosis, skin diseases such as psoriasis, excema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

The invention also relates to a method of treating a hyperproliferative disorder in a mammal which comprises administering to said mammal a therapeutically effective amount of the compound of formula 1, or a pharmaceutically acceptable salt, prodrug or hydrate thereof. In one embodiment, said method relates to the treatment of cancer such as brain, squamous cell, bladder, gastric, pancreatic, breast, head, neck, oesophageal, prostate, colorectal, lung, renal, kidney, ovarian, gynecological or thyroid cancer. In another embodiment, said method relates to the treatment of a non-cancerous hyperproliferative disorder such as benign hyperplasia of the skin (e.g., psoriasis) or prostate (e.g., benign prostatic hypertropy (BPH)).

The invention also relates to a method for the treatment of a hyperproliferative disorder in a mammal which comprises administering to said mammal a therapeutically effective amount of a compound of formula 1, or a pharmaceutically acceptable salt, prodrug or hydrate thereof, in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

The invention also relates to a method of treating pancreatitis or kidney disease in a mammal which comprises administering to said mammal a therapeutically effective amount of a compound of formula 1, or a pharmaceutically acceptable salt, prodrug or hydrate thereof.

The invention also relates to a method of preventing blastocyte implantation in a mammal which comprises administering to said mammal a therapeutically effective amount of a compound of formula 1, or a pharmaceutically acceptable salt, prodrug or hydrate thereof.

The invention also relates to a method of treating diseases related to vasculogenesis or angiogenesis in a mammal which comprises administering to said mammal an effective amount of a compound of formula 1, or a pharmaceutically acceptable salt, prodrug or hydrate thereof. In one embodiment, said method is for treating a disease selected from the group consisting of tumor angiogenesis, chronic inflammatory disease such as rheumatoid arthritis, atherosclerosis, skin diseases such as psoriasis, excema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

Patients that can be treated with a compounds of formula 1, and the pharmaceutically acceptable salts, prodrugs and hydrates of said compounds, according to the methods of this invention include, for example, patients that have been diagnosed as having psoriasis, BPH, lung cancer, bone cancer, pancreatic cancer, skin cancer, cancer of the head and neck, cutaneous or intraocular melanoma, uterine cancer, ovarian cancer, rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphonas, cancer of the bladder, cancer of the kidney or ureter (e.g., renal cell carcinoma, carcinoma of the renal pelvis), or neoplasms of the central nervous system (e.g., primary CNS lymphona, spinal axis tumors, brain stem gliomas or pituitary adenomas).

This invention also relates to a pharmaceutical composition for inhibiting abnormal cell growth in a mammal which comprises an amount of a compound of formula 1, or a pharmaceutically acceptable salt or solvate or prodrug thereof, in combination with an amount of a chemotherapeutic, wherein the amounts of the compound, salt, solvate, or prodrug, and of the chemotherapeutic are together effective in inhibiting abnormal cell growth. Many chemotherapeutics are presently known in the art. In one embodiment, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, e.g. anti-androgens.

This invention further relates to a method for inhibiting abnormal cell growth in a mammal which method comprises administering to the mammal an amount of a compound of formula 1, or a pharmaceutically acceptable salt or solvate or prodrug thereof, in combination with radiation therapy, wherein the amount of the compound, salt, solvate or prodrug is in combination with the radiation therapy effective in inhibiting abnormal cell growth in the mammal. Techniques for administering radiation therapy are known in the art, and these techniques can be used in the combination therapy described herein. The administration of the compound of the invention in this combination therapy can be determined as described herein.

It is believed that the compounds of formula 1 can render abnormal cells more sensitive to treatment with radiation for purposes of killing and/or inhibiting the growth of such cells. Accordingly, this invention further relates to a method for sensitizing abnormal cells in a mammal to treatment with radiation which comprises administering to the mammal an amount of a compound of formula 1 or pharmaceutically acceptable salt, prodrug or solvate thereof, which amount is effective in sensitizing abnormal cells to treatment with radiation. The amount of the compound, salt, or solvate in this method can be determined according to the means for ascertaining effective amounts of such compounds described herein.

This invention also relates to a pharmaceutical composition for inhibiting abnormal cell growth in a mammal, including a human, comprising an amount of a compound of the formula 1 as defined above, or a pharmaceutically acceptable salt, prodrug or solvate thereof, that is effective in inhibiting farnesyl protein transferase, and a pharmaceutically acceptable carrier.

This invention further relates to a pharmaceutical composition for inhibiting abnormal cell growth in a mammal comprising an amount of a compound of formula 1, or a pharmaceutically acceptable salt or solvate or prodrug thereof, that is effective in inhibiting abnormal cell growth, and a pharmaceutically acceptable carrier.

This invention also relates to a method of and to a pharmaceutical composition for inhibiting abnormal cell growth in a mammal which comprises an amount of a compound of formula 1, a pharmaceutically acceptable salt or solvate thereof, a prodrug thereof, or an isotopically-labelled derivative thereof, and an amount of one or more substances selected from anti-angiogenesis agents, signal transduction inhibitors, and antiproliferative agents.

This invention also relates to a pharmaceutical composition for inhibiting abnormal cell growth in a mammal, including a human, comprising an amount of a compound of formula 1 as defined above, or a pharmaceutically acceptable salt or solvate thereof, that is effective in inhibiting farnesyl protein transferase, and a pharmaceutically acceptable carrier.

This invention also relates to a method of and to a pharmaceutical composition for inhibiting abnormal cell growth in a mammal which comprises an amount of a compound of formula 1, a pharmaceutically acceptable salt or solvate thereof, a prodrug thereof, or an isotopically-labelled derivative thereof, and an amount of one or more substances selected from anti-angiogenesis agents, signal transduction inhibitors, and antiproliferative agents.

Anti-angiogenesis agents, such as MMP-2 (matrix-metalloprotienase 2) inhibitors, MMP-9 (matrix-metalloprotienase 9) inhibitors, and COX-II (cyclooxygenase II) inhibitors, can be used in conjunction with a compound of formula 1 and pharmaceutical compositions described herein. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Some specific examples of MMP inhibitors useful in the present invention are AG-3340, RO 32-3555, RS 13-0830, and the compounds recited in the following list:

3-[[4-(4-fluoro-phenoxy)-benzenesulfonyl]-(1-hydroxycarbamoyl-cyclopentyl)-amino]-propionic acid;

3-exo-3-[4-(4-fluoro-phenoxy)-benzenesulfonylamino]-8-oxa-bicyclo[3.2.1]octane-3-carboxylic acid hydroxyamide;

(2R, 3R) 1-[4-(2-chloro-4-fluoro-benzyloxy)-benzenesulfonyl]-3-hydroxy-3-methyl-piperidine-2-carboxylic acid hydroxyamide;

4-[4-(4-fluoro-phenoxy)-benzenesulfonylamino]-tetrahydro-pyran-4-carboxylic acid hydroxyamide;

3-[[4-(4-fluoro-phenoxy)-benzenesulfonyl]-(1-hydroxycarbamoyl-cyclobutyl)-amino]-propionic acid;

4-[4-(4-chloro-phenoxy)-benzenesulfonylamino]-tetrahydro-pyran-4-carboxylic acid hydroxyamide;

(R) 3-[4-(4-chloro-phenoxy)-benzenesulfonylamino]-tetrahydro-pyran-3-carboxylic acid hydroxyamide;

(2R, 3R) 1-[4-(4-fluoro-2-methyl-benzyloxy)-benzenesulfonyl]-3-hydroxy-3-methyl-piperidine-2-carboxylic acid hydroxyamide;

3-[[4-(4-fluoro-phenoxy)-benzenesulfonyl]-(1-hydroxycarbamoyl-1-methyl-ethyl)-amino]-propionic acid;

3-[[4-(4-fluoro-phenoxy)-benzenesulfonyl]-(4-hydroxycarbamoyl-tetrahydro-pyran-4-yl)-amino]-propionic acid;

3-exo-3-[4-(4-chloro-phenoxy)-benzenesulfonylamino]-8-oxa-bicyclo[3.2.1]octane-3-carboxylic acid hydroxyamide;

3-endo-3-[4-(4-fluoro-phenoxy)-benzenesulfonylamino]-8-oxa-bicyclo[3.2.1]octane-3-carboxylic acid hydroxyamide; and

(R) 3-[4-(4-fluoro-phenoxy)-benzenesulfonylamino]-tetrahydro-furan-3-carboxylic acid hydroxyamide;

and pharmaceutically acceptable salts and solvates of said compounds.

Other anti-angiogenesis agents, including other COX-II inhibitors and other MMP inhibitors, can also be used in the present invention.

A compound of formula 1 can also be used with signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors, such as VEGF receptors and molecules that can inhibit VEGF; and erbB2 receptor inhibitors, such as organic molecules or antibodies that bind to the erbB2 receptor, for example, HERCEPTIN™ (Genentech, Inc. of South San Francisco, Calif., USA).

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), and such substances can be used in the present invention as described herein. EGFR-inhibiting agents include, but are not limited to, the monoclonal antibodies C225 and anti-EGFR 22Mab (ImClone Systems Incorporated of New York, N.Y., USA), the compounds ZD-1839 (AstraZeneca), BIBX-1382 (Boehringer Ingelheim), MDX-447 (Medarex Inc. of Annandale, N.J., USA), and OLX-103 (Merck & Co. of Whitehouse Station, N.J., USA), VRCTC-310 (Ventech Research) and EGF fusion toxin (Seragen Inc. of Hopkinton, Mass.). These and other EGFR-inhibiting agents can be used in the present invention.

VEGF inhibitors, for example SU-5416 and SU-6668 (Sugen Inc. of South San Francisco, Calif., USA), can also be combined with the compound of the present invention. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are incorporated herein in their entireties by reference. Other examples of some specific VEGF inhibitors useful in the present invention are IM862 (Cytran Inc. of Kirkland, Wash., USA); anti-VEGF monoclonal antibody of Genentech, Inc. of South San Francisco, Calif.; and angiozyme, a synthetic ribozyme from Ribozyme (Boulder, Colo.) and Chiron (Emeryville, Calif.). These and other VEGF inhibitors can be used in the present invention as described herein.

ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), can furthermore be combined with the compound of the invention, for example those indicated in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), which are all hereby incorporated herein in their entireties by reference. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are incorporated in their entireties herein by reference. The erbB2 receptor inhibitor compounds and substance described in the aforementioned PCT applications, U.S. patents, and U.S. provisional applications, as well as other compounds and substances that inhibit the erbB2 receptor, can be used with the compound of the present invention in accordance with the present invention.

The compounds of the invention can also be used with other agents useful in treating abnormal cell growth or cancer, including, but not limited to, agents capable of enhancing antitumor immune responses, such as CTLA4 (cytotoxic lymphocite antigen 4) antibodies, and other agents capable of blocking CTLA4; and anti-proliferative agents such as other farnesyl protein transferase inhibitors, and the like. Specific CTLA4 antibodies that can be used in the present invention include those described in U.S. Provisional Application 60/113,647 (filed Dec. 23, 1998) which is incorporated by reference in its entirety, however other CTLA4 antibodies can be used in the present invention.

The compounds of the invention can also be used with other agents useful in treating abnormal cell growth or cancer, including, but not limited to, Primomastat (Agouron Pharmaceuticals, Inc.), Marimastat (British Biotech), Neovastat (Aeterna), Thalidomide (Celegene), Vitaxin (Medimmune), TNP470 (TAP Holdings), IMC-1C11 (ImClone Systems), CA4P (Oxigene) and Endostatin (EntreMed).

The subject invention also includes isotopically-labelled compounds, which are identical to those recited in formula 1 but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula 1 of this invention and prodrugs thereof can generally be prepared by carrying out the procedures disclosed in the Schemes and/or in the Examples below, by substituting a readily available isotopically labelled reagent for a non-isotopically labelled reagent.

The compounds of formula 1 and their pharmaceutically acceptable salts and solvates can each independently also furthermore be used in a palliative neo-adjuvant/adjuvant therapy in alleviating the symptoms associated with the diseases recited herein as well as the symptoms associated with abnormal cell growth. Such therapy can be a monotherapy or can be in a combination with chemotherapy and/or immunotherapy.

The terms “abnormal cell growth” and “hyperproliferative disorder” are used interchangeably in this application.

“Abnormal cell growth”, as used herein, refers to cell growth that is independent of normal regulatory mechanisms (e.g., loss of contact inhibition), including the abnormal growth of normal cells and the growth of abnormal cells. This includes, but is not limited to, the abnormal growth of: (1) tumor cells (tumors), both benign and malignant, expressing an activated Ras oncogene; (2) tumor cells, both benign and malignant, in which the Ras protein is activated as a result of oncogenic mutation in another gene; (3) benign and malignant cells of other proliferative diseases in which aberrant Ras activation occurs. Examples of such benign proliferative diseases are psoriasis, benign prostatic hypertrophy, human papilloma virus (HPV), and restinosis. “Abnormal cell growth” also refers to and includes the abnormal growth of cells, both benign and malignant, resulting from activity of the enzyme farnesyl protein transferase.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

The term “halo”, as used herein, unless otherwise indicated, means fluoro, chloro, bromo or iodo. Preferred halo groups are fluoro, chloro and bromo.

The term “alkyl”, as used herein, unless otherwise indicated, means saturated monovalent hydrocarbon radicals having straight, cyclic or branched moieties. Said “alkyl” group may include an optional carbon-carbon double or triple bond where said alkyl group comprises at least two carbon atoms. It is understood that for cyclic moieties at least three carbon atoms are required in said alkyl group.

The term “alkoxy”, as used herein, unless otherwise indicated, means O-alkyl groups wherein “alkyl” is as defined above.

The term “aryl”, as used herein, unless otherwise indicated, means an organic radical derived from an aromatic hydrocarbon by removal of one hydrogen, such as phenyl or naphthyl.

The term “5 to 10 membered heterocyclic” or “5 to 13 membered heterocyclic”, as used herein, unless otherwise indicated, means aromatic and non-aromatic heterocyclic groups containing one to four heteroatoms each selected from O, S and N, wherein each heterocyclic group has from 5 to 10 or 5 to 13 atoms in its ring system. The heterocyclic groups include benzo-fused ring systems and ring systems substituted with one or two oxo (═O) moieties such as pyrrolidin-2-one. An example of a 5 membered heterocyclic group is thiazolyl, an example of a 10 membered heterocyclic group is quinolinyl and an example of a 13 membered heterocyclic group is a carbazole group. Examples of non-aromatic heterocyclic groups are pyrrolidinyl, tetrahydrofuranyl, tetrahydrothienyl, tetrahydropyranyl, tetrahydrothiopyranyl, piperidino, morpholino, thiomorpholino, thioxanyl, piperazinyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, benzo[1,3]dioxolyl, quinazolinyl, quinoxalinyl, naphthyridinyl, and furopyridinyl. The foregoing groups, as derived from the compounds listed above, may be C-attached or N-attached where such is possible. For instance, a group derived from pyrrole may be pyrrol-1-yl (N-attached) or pyrrol-3-yl (C-attached).

The phrase “pharmaceutically acceptable salt(s)”, as used herein, unless otherwise indicated, includes salts of acidic or basic groups which may be present in the compounds of formula 1. The compounds of formula 1 that are basic in nature are capable of forming a wide variety of salts with various inorganic and organic acids. The acids that may be used to prepare pharmaceutically acceptable acid addition salts of such basic compounds of formula 1 are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts.

Those compounds of the formula 1 that are acidic in nature, are capable of forming base salts with various pharmacologically acceptable cations. Examples of such salts include the alkali metal or alkaline earth metal salts and particularly, the sodium and potassium salts.

The compounds of the present invention have asymmetric centers and therefore exist in different enantiomeric and diastereomeric forms. This invention relates to the use of all optical isomers and stereoisomers of the compounds of the present invention, and mixtures thereof, and to all pharmaceutical compositions and methods of treatment that may employ or contain them. The compounds of formula 1 may also exist as tautomers. This invention relates to the use of all such tautomers and mixtures thereof.

This invention also encompasses pharmaceutical compositions containing and methods of treating proliferative disorders or abnormal cell growth through administering prodrugs of compounds of the formula 1. Compounds of formula 1 having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula 1. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

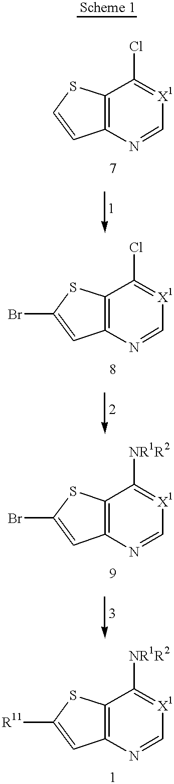

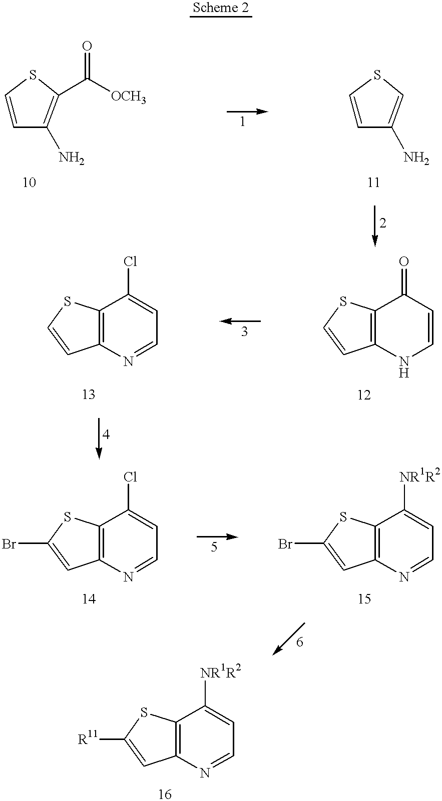

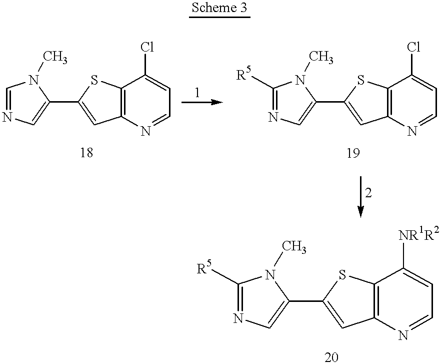

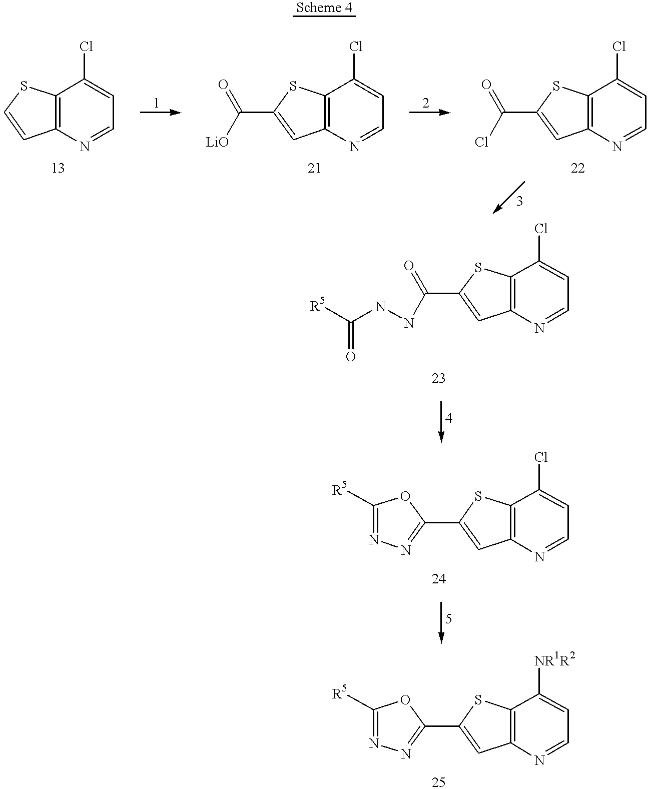

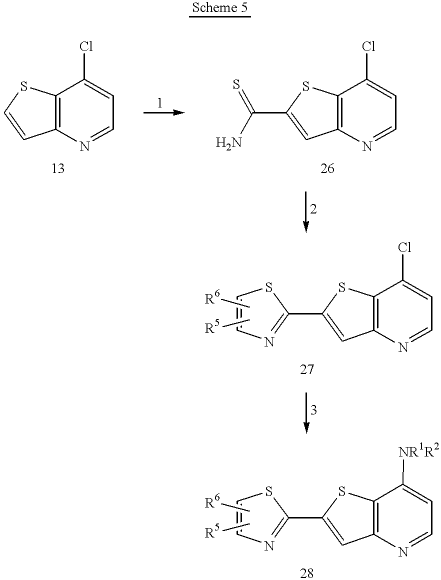

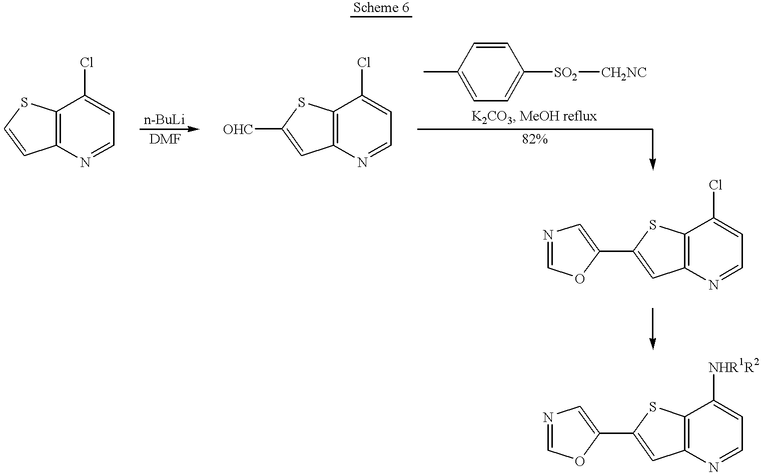

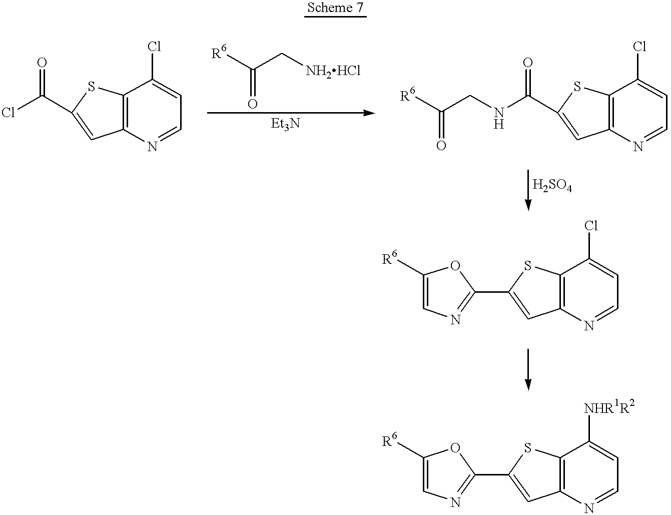

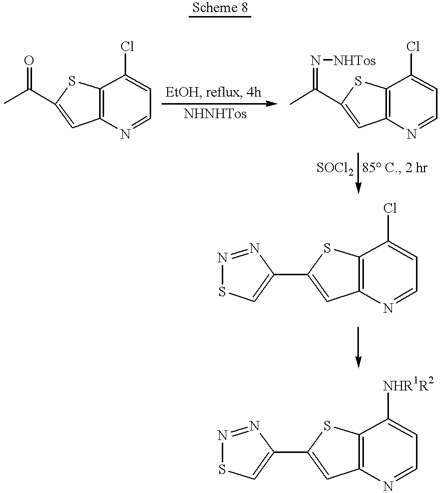

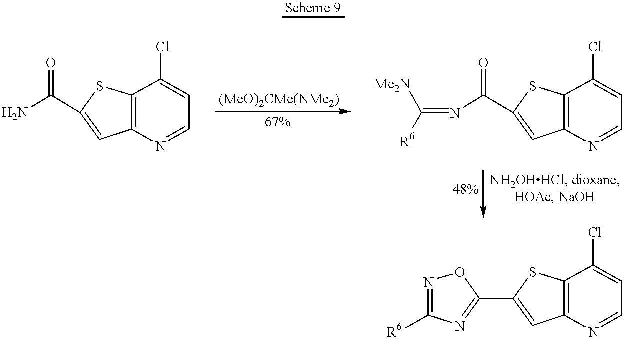

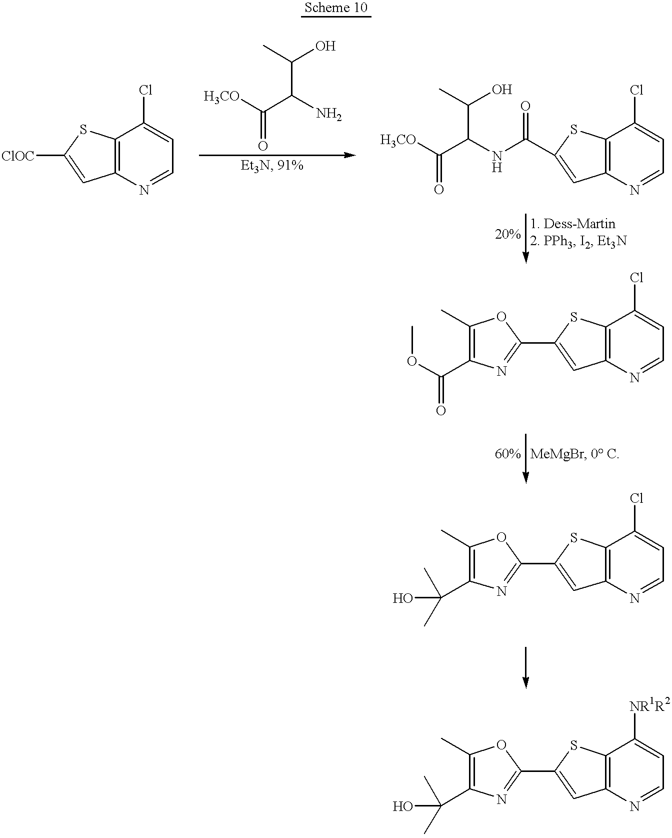

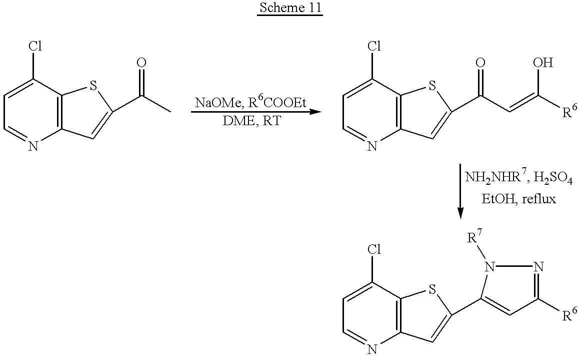

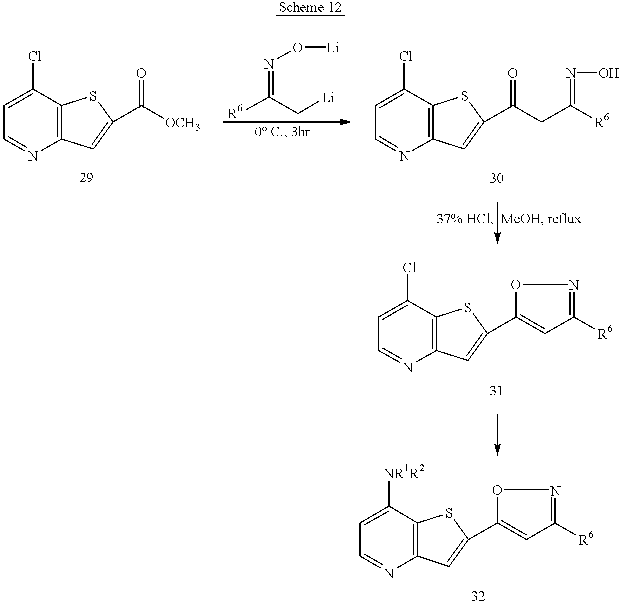

### DETAILED DESCRIPTION OF THE INVENTION

The preparation of the compounds of the present invention is illustrated in Schemes 1-12

The compounds of the present invention are readily prepared according to synthetic methods familiar to those skilled in the art. Scheme 1 illustrates a general synthetic procedure for preparing the compounds of the present invention. The compound of formula 7 (in which X¹ is as defined above) may be prepared by one or more procedures described in published PCT international applications numbers WO 95/19774 (published Jul. 27, 1995), WO 95/19970 (published Jul. 27, 1995), and WO 97/13771 (published Apr. 17, 1997). In addition, 4-chlorothieno[3,2-d]pyrimidine is commercially available, such as from Maybridge Chemical Co. Ltd. A preferred method of preparing 4-chlorothieno[3,2-d]pyridine is described below with reference to steps 1-3 of Scheme 2.

In step 1 of Scheme 1, the compound of formula 7 may be converted to the corresponding bromo derivative of formula 8 by treating the starting compound with lithium diisopropylamine or n-butyllithium, and then 1,2-dibromo-1,1,2,2-tetrafluoroethane or bromine in a non-polar solvent, such as tetrahydrofuran (THF), at a temperature of about −78° C. for a period of about 15 minutes to one-half hour and then gradually warming the mixture to room temperature (20-25° C.).

In step 2 of Scheme 1, the compound of formula 8 may be coupled with a compound of formula HNR¹R², wherein R¹ and R² are as defined above, optionally in the presence of a base, such as pyridine, triethylamine or sodium hydride, and optionally in the presence of pyridine hydrochloride as a catalyst, under an inert atmosphere, such as dry nitrogen gas, in a solvent, such as a C₁-C₆ alcohol, dimethylformamide (DMF), 1,2-dichloroethane (DCE), N-methylpyrrolidin-2-one (NMP), chloroform, acetonitrile, tetrahydrofuran (THF), dimethylsulfoxide (DMSO), 1,4-dioxane or pyridine, or a mixture of two or more of the foregoing solvents, preferably a mixture of t-butyl alcohol and DCE, at a temperature of from ambient to reflux temperature, preferably 80-125° C., for a period of about 2 hours to 72 hours to provide the compound of formula 9. The foregoing reaction is preferably done in a sealed tube.

Where the compound of formula HNR¹R² is an optionally substituted indole or indoline moiety, such compounds can be prepared according to one or more methods known to those skilled in the art. Such methods are described in PCT international patent application publication number WO 95/23141, referred to above, and in W. C. Sumpter and F. M. Miller, “Heterocyclic Compounds with Indole and Carbazole Systems,” in volume 8 of “The Chemistry of Heterocyclic Compounds”, Interscience Publishers Inc., New York (1954). Optional substituents can be included as appropriate before or after the coupling step illustrated in Scheme 1. Prior to the coupling step, primary and secondary amino moieties (other than said amine of formula HNR¹R²) are preferably protected using a nitrogen protecting group known to those skilled in the art. Such protecting groups and their use are described in T. W. Greene and P. G. M. Wuts, “Protective Groups in Organic Synthesis,” Second Edition, John Wiley & Sons, New York, 1991.

In step 3 of Scheme 1, the compound of formula 9 may be converted to the compound of formula 1 by coupling the starting compound with a compound of the formula R¹¹—B(OH)₂ (wherein R¹¹ is as defined above) in the presence of 1,4-bis(diphenylphosphino)butane and a palladium catalyst, such as bis(benzonitrile)-palladium(II) chloride, a base, such as sodium or potassium carbonate, and a solvent, such as toluene, ethanol, THF, DMF, or dimethoxyethane (DME), preferably a mixture of toluene, ethanol and THF, at a temperature within the range of about 50-110° C. for a period of about 1 to 24 hours. This step is analogous to the Suzuki coupling procedure described in N. Miyaura, A. Suzuki, Chem. Rev. 1995, 95, 2457.

In the alternative, steps 2 and 3 of Scheme 1 may be reversed. That is, the R¹¹ group may be introduced into the compound of formula 7 followed by the coupling of the resulting compound with the compound of formula HNR¹R² as described above.

In another procedure, step 3 of Scheme 1 may be achieved by reacting the compound of formula 9 with a compound of the formula (trialkylstannyl)-R¹¹ (wherein R¹¹ is as defined above), such as (tributylstannyl)-R¹¹, in the presence of copper iodide and trans-benzyl(chloro)bis(triphenylphosphine)palladium(II) in DMF at a temperature of about 90° C. for a period of about 14 hours. The starting compound for this procedure, specifically (tributylstannyl)-R¹¹, may be prepared from R¹¹—Br by at least three separate procedures. In a first procedure, R¹¹—Br may be treated with (tributylstannyl)-chloride and n-butyllithium in THF or DMF to provide (tributylstannyl)-R¹¹. In a second procedure, R¹¹—Br may be treated with Bu₃Sn—SnBu₃, wherein Bu represents butyl, and sodium metal to provide (tributylstannyl)-R¹¹. And in a third procedure, R¹¹—Br may be treated with Bu₃Sn—SnBu₃, wherein Bu represents butyl, and Pd(PPh₃)₄, wherein Ph represents phenyl, in toluene to provide (tributylstannyl)-R¹¹.

Following or before step 3 of Scheme 1, the R¹¹ group may be modified to introduce one or more R⁵ groups (wherein R⁵ is as defined above). In a one preferred method, where R¹¹ is a heteroaryl group that includes an aldehyde group, the aldehyde may be converted to a preferred aminomethyl group. In this process, the starting compound that includes an aldehyde on the R¹¹ group is reacted with an amine of the formula HNR⁶R⁷ (wherein R⁶ and R⁷ are as defined above) in the presence of a reducing agent, such as sodium cyanoborohydride or sodium borohydride, in a solvent comprising acetic acid and ethanol or methanol at a temperature in the range of 0-100° C., preferably room temperature. This process converts the aldehyde to a moiety of the formula R⁶R⁷NCH₂—.″) Other methods of modifying the compounds of formula 1 will be obvious to those skilled in the art.

Scheme 2 illustrates a procedure for preparing the compounds of formula 1 wherein X is CH. In step 1 of Scheme 2, the compound of formula 10 (3-amino-thiophene-2-carboxylic acid methyl ester) is dissolved in sodium hydroxide and refluxed for about 2 hours. The solution is then cooled to 0° C. and acidified to pH 5 with concentrated HCl at which time a precipitate will form. The precipitate is separated and treated with propanol and oxalic acid, and the solution is stirred at about 38° C. for approximately 45 minutes to provide the compound of formula 11 (thiophen-3-ylamine). In step 2 of Scheme 2, the compound of formula 11 is dissolved in triethyl orthoformate and stirred at room temperature until dissolution is complete. 2,2-Dimethyl-[1,3]dioxane-4,6-dione is then added portionwise at room temperature, with a precipitate forming upon completion of the addition. The mixture is then heated at 85° C. overnight. The resulting precipitate, which is an intermediate (2,2-dimethyl-5-(thiophen-3-ylaminomethylene)-[1,3]dioxane-4,6-dione), is then separated and washed. The intermediate is added to dowtherm A (heated to 260° C.), and the resulting mixture is heated for 30 minutes and then cooled to room temperature to provide the compound of formula 12. In step 3 of Scheme 2, the compound of formula 12 is added to oxalyl chloride in a mixture of methylene chloride and DMF and heated to reflux for approximately two hours to provide the compound of formula 13. The compound of formula 13 may be converted to the compound of formula 14 as described above with respect to step 1 of Scheme 1. The compound of formula 14 may be converted to the compound of formula 15 as described above with respect to step 2 of Scheme 1. The compound of formula 15 may be converted to the compound of formula 16 as described above with respect to step 3 of Scheme 1.

Scheme 3 illustrates a procedure for preparing the compounds of formula 1 wherein X is CH and R¹¹ is substituted Imidiazole. The compound of formula 18 is prepared as described in WO 99/2440, hereby incorporated by reference. Examples 1-24 provide representative Examples of this synthetic scheme.

Scheme 4 illustrates a procedure for preparing the compounds of formula 1 wherein X is CH and R¹¹ is substituted oxadiazole. A representative Example of Scheme 3 is detailed in Example 25.

Scheme 5 illustrates a procedure for preparing the compounds of formula 1 wherein X is CH and R¹¹ is substituted thiazol. Examples 26-32 provide a variety of Examples employing the synthetic scheme illustrated in Scheme 5.

Scheme 6 illustrates a procedure for preparing the compounds of formula 1 wherein X is CH and R¹¹ is oxazole. Example 42 employs this synthetic scheme.

Scheme 7 illustrates a procedure for preparing the compounds of formula 1 wherein X is CH and R¹¹ is a substituted oxazole. Example 43 employs this synthetic scheme.

Scheme 8 illustrates a procedure for preparing compounds of the formula 1 wherein X is CH and R¹¹ is a thiadiazole. In this procedure, treatment of the methyl ketone with tosyl hydrazine under dehydrating conditions affords the tosyl hydrazone. Cyclization is achieved by treatment of the hydrazone with thionyl chloride, and the HNR¹R² group is introduced as described above.

Scheme 9 illustrates a procedure for preparing compounds of the formula 1 wherein X is CH and R¹¹ is a 1,2,4-oxadiazole. Treatment of the amide with an acetal such as dimethylamino acetone dimethyl acetal affords a product that can be treated with ammonium hydroxide to afford the oxadiazole. Introduction of the group HNR¹R² proceeds as described above.

Scheme 10 illustrates a procedure for the synthesis of optionally substituted oxazole derivatives. In this case, addition of threonine methyl ester to the acid chloride affords a product that can be oxidized, for example with the Dess-Martin periodinane, and cyclized upon treatment with iodine and triphenylphosphine. Introduction of the group HNR¹R² is carried out as described above, and the ester substituent can be transformed—either before or after the introduction of the group HNR¹R²—by methods known to one skilled in the art to a variety of optional groups. For example, treatment of the ester with methyl magnesium bromide at low temperature in an inert solvent such as THF affords the tertiary alcohol shown in the scheme.

Scheme 11 illustrates a procedure for the synthesis of optionally substituted pyrazole derivatives. Reaction of the methyl ketone with base and an ester affords 1,3-diketones. Subsequent reaction with an optionally substituted hydrazine derivative affords pyrazole-substituted thienopyridines. Introduction of the group HNR¹R² then proceeds as described above.

Scheme 12 illustrates a procedure for the synthesis of optionally substituted isoxazole derivatives. Reaction of the methyl ester of formula 29 with a bis-metallated oxime (which may be optionally substituted, for example with an R⁶ substitutent) affords a compound of formula 30. Treatment of the compound of formula 30 with a mineral acid such as 37% HCl in a solvent, such as methanol, affords cyclized product of formula 31. Introduction of the group HNR¹R² then proceeds as described above resulting in the compound of formula 32.

The compounds of the present invention may have asymmetric carbon atoms. Such diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomric mixture by reaction with an appropriate optically active compound (e.g., alcohol), separating the diastereomers and converting (e.g., hydrolyzing) the individual diastereomers to the corresponding pure enantiomers. All such isomers, including diastereomer mixtures and pure enantiomers are considered as part of the invention.

The compounds of formula 1 that are basic in nature are capable of forming a wide variety of different salts with various inorganic and organic acids. Although such salts must be pharmaceutically acceptable for administration to animals, it is often desirable in practice to initially isolate the compound of formula 1 from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of formula 1 that are acidic in nature, are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of formula 1. Such non-toxic base salts include those derived from such pharmacologically acceptable cations as sodium, potassium calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

The compounds of the present invention are potent inhibitors of the erbB family of oncogenic and protooncogenic protein tyrosine kinases such as epidermal growth factor receptor (EGFR), erbB2, HER3, or HER4 and thus are all adapted to therapeutic use as antiproliferative agents (e.g., anticancer) in mammals, particularly in humans. The compounds of the present invention are also inhibitors of angiogenesis and/or vasculogenesis. In particular, the compounds of the present invention are useful in the prevention and treatment of a variety of human hyperproliferative disorders such as malignant and benign tumors of the liver, kidney, bladder, breast, gastric, ovarian, colorectal, prostate, pancreatic, lung, vulval, thyroid, hepatic carcinomas, sarcomas, glioblastomas, head and neck, and other hyperplastic conditions such as benign hyperplasia of the skin (e.g., psoriasis) and benign hyperplasia of the prostate (e.g., BPH). It is, in addition, expected that a compound of the present invention may possess activity against a range of leukemias and lymphoid malignancies.

The compounds of the present invention may also be useful in the treatment of additional disorders in which aberrant expression ligand/receptor interactions or activation or signalling events related to various protein tyrosine kinases, are involved. Such disorders may include those of neuronal, glial, astrocytal, hypothalamic, and other glandular, macrophagal, epithelial, stromal, and blastocoelic nature in which aberrant function, expression, activation or signalling of the erbB tyrosine kinases are involved. In addition, the compounds of the present invention may have therapeutic utility in inflammatory, angiogenic and immunologic disorders involving both identified and as yet unidentified tyrosine kinases that are inhibited by the compounds of the present invention.

The in vitro activity of the compounds of formula 1 in inhibiting the receptor tyrosine kinase (and thus subsequent proliferative response, e.g., cancer) may be determined by the following procedure. The activity of the compounds of formula 1 in vitro, can be determined by the amount of inhibition of the phosphorylation of an exogenous substrate (e.g., Lys₃-Gastrin or polyGluTyr (4:1) random copolymer (I. Posner et al., J. Biol. Chem. 267 (29), 20638-47 (1992)) on tyrosine by epidermal growth factor receptor kinase by a test compound relative to a control. Affinity purified, soluble human EGF receptor (96 ng) is obtained according to the procedure in G. N. Gill, W. Weber, Methods in Enzymology 146, 82-88 (1987) from A431 cells (American Type Culture Collection, Rockville, Md.) and preincubated in a microfuge tube with EGF (2 μg/ml) in phosphorylation buffer+vanadate (PBV: 50 mM HEPES, pH 7.4; 125 mM NaCl; 24 mM MgCl₂; 100 μM sodium orthovanadate), in a total volume of 10 μl, for 20-30 minutes at room temperature. The test compound, dissolved in dimethylsulfoxide (DMSO), is diluted in PBV, and 10 μl is mixed with the EGFR/EGF mix, and incubated for 10-30 minutes at 30° C. The phosphorylation reaction is initiated by addition of 20 μl ³³P-ATP/ substrate mix (120 μM Lys₃-Gastrin (sequence in single letter code for amino acids, KKKGPWLEEEEEAYGWLDF), 50 mM Hepes pH 7.4, 40 μM ATP, 2 μCi γ-[³³P]-ATP) to the EGFr/EGF mix and incubated for 20 minutes at room temperature. The reaction is stopped by addition of 10 μl stop solution (0.5 M EDTA, pH 8; 2mM ATP) and 6 μl 2N HCl. The tubes are centrifuged at 14,000 RPM, 4° C., for 10 minutes. 35 μl of supernatant from each tube is pipetted onto a 2.5 cm circle of Whatman P81 paper, bulk washed four times in 5% acetic acid, 1 liter per wash, and then air dried. This results in the binding of substrate to the paper with loss of free ATP on washing. The [³³P] incorporated is measured by liquid scintillation counting. Incorporation in the absence of substrate (e.g., lys₃-gastrin) is subtracted from all values as a background and percent inhibition is calculated relative to controls without test compound present. Such assays, carried out with a range of doses of test compounds, allow the determination of an approximate IC₅₀ value for the in vitro inhibition of EGFR kinase activity.

The activity of the compounds of formula 1 in vivo, can be determined by the amount of inhibition of tumor growth by a test compound relative to a control. The tumor growth inhibitory effects of various compounds are measured according to the methods of Corbett T. H., et al. “Tumor Induction Relationships in Development of Transplantable Cancers of the Colon in Mice for Chemotherapy Assays, with a Note on Carcinogen Structure”, Cancer Res., 35, 2434-2439 (1975) and Corbett, T. H., et al., “A Mouse Colon-tumor Model for Experimental Therapy”, Cancer Chemother. Rep. (Part 2)”, 5, 169-186 (1975), with slight modifications. Tumors are induced in the left flank by s.c. injection of 1×10⁶ log phase cultured tumor cells (human MDA-MB-468 breast or human HN5 head and neck carcinoma cells) suspended in 0.10 ml RPMI 1640. After sufficient time has elapsed for the tumors to become palpable (2-3 mm in diameter) the test animals (athymic mice) are treated with active compound (formulated by dissolution in DMSO typically at a concentration of 50 to 100 mg/mL followed by 1:9 dilution into saline or, alternatively, 1:9 dilution into 0.1% Pluronic™ P105 in 0.9% saline) by the intraperitoneal (ip) or oral (po) routes of administration twice daily (i.e., every 12 hours) for 5 consecutive days. In order to determine an anti-tumor effect, the tumor is measured in millimeters with Vernier calipers across two diameters and the tumor size (mg) is calculated using the formula: Tumor weight=(length×[width]²)/2, according to the methods of Geran, R. I., et al. “Protocols for Screening Chemical Agents and Natural Products Against Animal Tumors and Other Biological Systems”, Third Edition, Cancer Chemother. Rep., 3, 1-104 (1972). Results are expressed as percent inhibition, according to the formula: Inhibition (%)=(TuW_{control}−TuWₜₑₛₜ)/TuW_{control}×100%. The flank site of tumor implantation provides reproducible dose/response effects for a variety of chemotherapeutic agents, and the method of measurement (tumor diameter) is a reliable method for assessing tumor growth rates.

Other methods of assessing the activity of the compounds of the present invention are referred to in PCT international application publication number WO 95/21613 (published Aug. 17, 1995) which is incorporated herein by reference.

The in vitro activity of the compounds of formula 1 in inhibiting the KDR/VEGF receptor may be determined by the following procedure.

The ability of the compounds of the present invention to inhibit tyrosine kinase activity may be measured using a recombinant enzyme in an assay that measures the ability of compounds to inhibit the phosphorylation of the exogenous substrate, polyGluTyr (PGT, Sigma™, 4:1). The kinase domain of the human KDR/VEGF receptor (amino acids 805-1350) is expressed in Sf9 insect cells as a glutathione S-transferase (GST)-fusion protein using the baculovirus expression system. The protein is purified from the lysates of these cells using glutathione agarose affinity columns. The enzyme assay is performed in 96-well plates that are coated with the PGT substrate (0.625 μg PGT per well). Test compounds are diluted in dimethylsulfoxide (DMSO), and then added to the PGT plates so that the final concentration of DMSO in the assay is 1.6% (v/v). The recombinant enzyme is diluted in phosphorylation buffer (50 mM Hepes, pH 7.3, 125 mM NaCl, 24 mM MgCl₂). The reaction is initiated by the addition of ATP to a final concentration of 10 μM. After a 30 minute incubation at room temperature with shaking, the reaction is aspirated, and the plates are washed with wash buffer (PBS-containing 0.1% Tween-20). The amount of phosphorylated PGT is quantitated by incubation with a HRP-conjugated (HRP is horseradish peroxidase) PY-54 antibody (Transduction Labs), developed with TMB peroxidase (TMB is 3,3′,5,5′-tetramethylbenzidine), and the reaction is quantitated on a BioRad™ Microplate reader at 450 nM. Inhibition of the kinase enzymatic activity by the test compound is detected as a reduced absorbance, and the concentration of the compound that is required to inhibit the signal by 50% is reported as the IC₅₀ value for the test compound.

To measure the ability of the compounds to inhibit KDR tyrosine kinase activity for the full length protein that exists in a cellular context, the porcine aortic endothelial (PAE) cells transfected with the human KDR (Waltenberger et al., J. Biol. Chem. 269:26988, 1994) may be used. Cells are plated and allowed to attach to 96-well dishes in the same media (Ham's F12) with 10% v/v FBS (fetal bovine serum). The cells are then washed, re-fed with serum depleted media (0.1% v/v FBS) that contains 0.1% (v/v) bovine serum albumin (BSA), and allowed to incubate for 16-24 hours. Immediately prior to dosing with compound, the cells are re-fed with the serum depleted media (0.1% v/v FBS) (without BSA). Test compounds, dissolved in DMSO, are diluted into the media (final DMSO concentration 0.5% (v/v)). At the end of a 2 hour incubation, VEGF₁₆₅ (50 ng/ml final) is added to the media for an 8 minute incubation. The cells are washed and lysed in 50μ lysis buffer containing 20 mM Tris-HCL (pH 8), 150 mM NaCl, 1% v/v NP40, 2 mM NaVO₄, 500 μM EDTA, 1 mM PMSF, and 1 tablet/25 ml EDTA free complete® Protease Inhibitor Table, Roche. The cell lysates is then diluted to a final volume of 150 μl in PBS/1 mM NaVO₄. The extent of phosphorylation of KDR is measured using an ELISA assay. Reactibind Goat-anti Rabbit plates (Pierce) are blocked with Superblock buffer (Pierce) prior to addition of the anti-flk-1 C-20 antibody (0.5 μg per well, Santa Cruz). Any unbound antibody is washed off the plates prior to addition of 100 μl cell lysate. After a 2 hour incubation of the lysates with the flk-1 antibody, the KDR associated phosphotyrosine is quantitated by development with the HRP-conjugated PY-54 antibody and TMB, as described above. The ability of the compounds to inhibit the VEGF-stimulated autophosphorylation reaction by 50%, relative to VEGF-stimulated controls is reported as the IC₅₀ value for the test compound.

The ability of the compounds to inhibit mitogenesis in human endothelial cells is measured by their ability to inhibit ³H-thymidine incorporation into HUVE cells (human umbilical vein endothelial cells, Clonetics™). This assay has been well described in the literature (Waltenberger J et al. J. Biol. Chem. 269: 26988, 1994; Cao Y et al. J. Biol. Chem. 271: 3154, 1996). Briefly, 10⁴ cells are plated in collagen-coated 24-well plates and allowed to attach. Cells are re-fed in serum-free media, and 24 hours later are treated with various concentrations of compound (prepared in DMSO, final concentration of DMSO in the assay is 0.2% v/v), and 2-30 ng/ml VEGF₁₆₅. During the last 3 hours of the 24 hour compound treatment, the cells are pulsed with ³H thymidine (NEN, 1 μCi per well). The media are then removed, and the cells washed extensively with ice-cold Hank's balanced salt solution, and then 2 times with ice cold trichloroacetic acid (10% v/v). The cells are lysed by the addition of 0.2 ml of 0.1 N NaOH, and the lysates transferred into scintillation vials. The wells are then washed with 0.2 ml of 0.1 N HCl, and this wash is then transferred to the vials. The extent of ³H thymidine incorporation is measured by scintillation counting. The ability of the compounds to inhibit incorporation by 50%, relative to control (VEGF treatment with DMSO vehicle only) is reported as the IC₅₀ value for the test compound.

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the active compound administered will be dependent on the subject being treated, the severity of the disorder or condition, the rate of administration and the judgement of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The active compound may be applied as a sole therapy or may involve one or more other anti-tumour substances, for example those selected from, for example, mitotic inhibitors, for example vinblastine; alkylating agents, for example cis-platin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-[N-(3,4-dihydro-2-methyl4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-trifluoromethyl) propionanilide). Such conjoint treatment may be achieved by way of the simultaneous, sequential or separate dosing of the individual components of the treatment.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The examples and preparations provided below further illustrate and exemplify the compounds of the present invention and methods of preparing such compounds. It is to be understood that the scope of the present invention is not limited in any way by the scope of the following examples and preparations.

Where HPLC chromatography is referred to in the preparations and examples below, the general conditions used, unless otherwise indicated, are as follows. The column used is a ODS Hypersil column (manufactured by Hewlett Packard) of 150 mm length and 4.0 mm interior diameter. The samples are run on a Hewlett Packard-1050 system. A gradient solvent method is used running 100 percent ammonium acetate/acetic acid buffer (0.2 M) to 100 percent acetonitrile over 10 minutes. The system then proceeds on a wash cycle with 100 percent acetonitrile for 1.5 minutes and then 100 percent buffer solution for 3 minutes. The flow rate over this period is a constant 3 ml minute.

### EXAMPLE 1

### 1-Methyl-5-[7-(2-methyl-1H-indol-5-ylamino)-thieno[3,2-b]pyridin-2-yl]-1H-imidazole-2-carboxylic Acid Methyl Ester

A. A solution of 7-chloro-2-(3-methyl-3H-imidazol-4-yl)-thieno[3,2-b]pyridine (250 mg, 1.0 mmol) in anhydrous tretrahydrofuran (THF) (30 mL) was cooled to −78° C. via dry ice/acetone bath. n-butyllithium (“n-BuLi”) (2.5 M in hexanes, 440 uL, 1.1 mmol) was added slowly. The solution was stirred at −78° C. for 30 minutes. Methyl chloroformate (190 mg, 2.0 mmol) was added dropwise. The heterogeneous reaction mixture was stirred at −78° C. for 30 minutes and the dry ice/acetone bath was removed. After warming to room temperature, the reaction mixture was treated with methanol. The crude material was concentrated onto 1 gram of silica gel and purified by chromatography through a Biotash Flash40M cartridge, eluting with dichloromethane/methanol (100/3 v/v) to afford 5-(7-chloro-thieno[3,2-b]pyridin-2-yl)-1-methyl-1H-imidazole-2-carboxylic acid methyl ester as a white solid (185 mg, 60%). MS: 308, 310 (MH+); HPLC Rf: 4.77 min; HPLC purity: 95%.

B. 5-(7-chloro-thieno[3,2-b]pyridin-2-yl)-1-methyl-1H-imidazole-2-carboxylic acid methyl ester (185 mg, 0.60 mmol) and 2-methyl-1H-indol-5-ylamine (105 mg, 0.72 mmol) was heated at reflux in ethanol for 48 hours. The solution was cooled to room temperature, concentrated onto silica gel, and purified by flash chromatography eluting with dichloromethane/methanol/triethylamine (100/6/1 v/v/v) to afford the title compound as a yellow solid (210 mg, 86%). MS: 418 (MH+); HPLC Rf: 4.84 min; HPLC purity:99%.

### EXAMPLE 2

### 1-Methyl-5-[7-(2-methyl-1H-indol-5-ylamino)-thieno[3,2-b]pyridin-2-yl]-1H-imidazole-2-carbonitrile

A. A solution of 7-chloro-2-(3-methyl-3H-imidazol-4-yl)-thieno[3,2-b]pyridine (250 mg, 1.0 mmol) in anhydrous THF (30 mL) was cooled to −78° C. via dry ice/acetone bath. n-BuLi (2.5 M in hexanes, 440 uL, 1.1 mmol) was added slowly, and the resulting solution was stirred at −78° C. for 30 minutes. p-Toluenesulfonyl cyanide (363 mg, 2.0 mmol) was added, and the reaction mixture was stirred at −78° C. for 30 minutes. The reaction mixture was allowed to warm to room temperature and was diluted with methanol. The crude material was concentrated onto 1 gram of silica gel and the purified by flash chromatography, eluting with dichloromethane/methanol (100/3 v/v) to give 5-(7-chloro-thieno[3,2-b]pyridin-2-yl)-1-methyl-1H-imidazole-2-carbonitrile as a white solid (242 mg, 88%). MS: 275, 277 (MH+), HPLC Rf: 5.06 min; HPLC purity: 97%.

B. The title compound (144 mg, 75%) was prepared from 5-(7-chloro-thieno[3,2-b]pyridin-2-yl)-1-methyl-1H-imidazole-2-carbonitrile and 2-methyl-1H-indol-5-ylamine by a procedure analogous to Example 1. MS: 385 (MH+); HPLC Rf: 4.72 min; HPLC purity: 90%.

### EXAMPLE 3

### {1-Methyl-5-[7-(2-methyl-1H-indol-5-ylamino)-thieno[3,2-b]pyridin-2-yl]-1H-imidazol-2-yl}-morpholin-4-yl-methanone

A. A solution of morpholine (92 mg, 0.30 mmol) in benzene (15 mL) was cooled to 0 C. Trimethylaluminum solution (2.0M in toulene, 300 uL, 0.60 mmol) was added dropwise. The resulting solution was warmed to room temperature and stirred for 1 hour. 5-(7-Chloro-thieno[3,2-b]pyridin-2-yl)-1-methyl-1H-imidazole-2-carboxylic acid methyl ester was added and the reaction mixture was heated at reflux for 4 hours. After cooling the reaction to room temperature 1 M hydrochloric acid (“HCl”) was added slowly, dropwise to quench the reaction. The reaction mixture was poured into 50 mL of 1 M HCl solution and extracted with 50 mL of ethyl acetate to remove organic impurities. The aqueous layer was taken to pH=8 with saturated sodium bicarbonate solution and was extracted with ethyl acetate. The combined organic phases from the second extraction were washed with brine and dried over sodium sulfate. After removing solvent in vacuo, [5-(7-chloro-thieno[3,2-b]pyridin-2-yl)-1-methyl-1H-imidazol-2-yl]-morpholin-4-yl-methanone was obtained as a white solid (98 mg, 0.27 mmol, 90%). MS: 363, 365 (MH+); HPLC Rf: 4.47 min; HPLC purity 99%.

B. [5-(7-Chloro-thieno[3,2-b]pyridin-2-yl)-1-methyl-1H-imidazol-2-yl]-morpholin-4-yl-methanone (98 mg, 0.27 mmol) and 2-methyl-1H-indol-5-ylamine (44 mg, 0.30 mmol) was heated at reflux in ethanol for 48 hours. The reaction mixture was cooled to room temperature and concentrated onto silica gel. Purification by flash chromatography through a short pad of silica gel eluting with dichloromethane/methanol/triethylamine (100/6/1) afforded the title compound as a yellow solid (113 mg, 90%). MS: 473 (MH+); HPLC Rf: 4.01 min; HPLC purity: 95%.

### EXAMPLE 4

### {1-Methyl-5-[7-(2-methyl-1H-indol-5-ylamino)-thieno[3,2-b]pyridin-2-yl]-1H-imidazol-2-yl}-piperidin-1-yl-methanone

A. [5-(7-Chloro-thieno[3,2-b]pyridin-2-yl)-1-methyl-1H-imidazol-2-yl]-piperidin-1-yl-methanone was prepared from 5-(7-chloro-thieno[3,2-b]pyridin-2-yl)-1-methyl-1H-imidazole-2-carboxylic acid methyl ester and piperidine by a procedure analogous to Example 3. MS: 361, 363 (MH+); HPLC Rf: 5.24 min; HPLC purity 97%.

B. The title compound was prepared from [5-(7-chloro-thieno[3,2-b]pyridin-2-yl)-1-methyl-1H-imidazol-2-yl]-piperidin-1-yl-methanone and 2-methyl-1H-indol-5-ylamine by a procedure analogous to Example 3. MS: 471 (MH+); HPLC Rf: 4.86 min; HPLC purity: 96%.

### EXAMPLE 5

### {1-Methyl-5-[7-(2-methyl-1H-indol-5-ylamino)-thieno[3,2-b]pyridin-2-yl]-1H-imidazol-2-yl}-(4-methyl-piperazin-1-yl)-methanone

A. [5-(7-Chloro-thieno[3,2-b]pyridin-2-yl)-1-methyl-1H-imidazol-2-yl]-(4-methyl-piperazin-1-yl)-methanone was prepared from 5-(7-chloro-thieno[3,2-b]pyridin-2-yl)-1-methyl-1H-imidazole-2-carboxylic acid methyl ester and N-methyl piperazine by a procedure analogous to Example 3. MS: 376, 378 (MH+); HPLC Rf: 4.00 min; HPLC purity: 95%.

B. The title compound was prepared from [5-(7-chloro-thieno[3,2-b]pyridin-2-yl)-1-methyl-1H-imidazol-2-yl]-(4-methyl-piperazin-1-yl)-methanone and 2-methyl-1H-indol-5-ylamine by a procedure analogous to Example 3. MS: 486 (MH+); HPLC Rf: 3.79 min; HPLC purity: 99%.

### EXAMPLE 6

### [2-(2-Isopropenyl-3-methyl-3H-imidazol-4-yl)-thieno[3,2-b]pyridin-7-yl]-(2-methyl-1H-indol-5-yl)-amine

A. To a suspension of 2-[5-(7-chloro-thieno[3,2-b]pyridin-2-yl)-1-methyl-1H-imidazol-2-yl]-propan-2-ol (WO 99/24440) (155 mg, 0.50 mmol) in toluene (10 mL), thionyl chloride solution (2.0 M in hexanes, 2.5 mL, 5 mmol) was added. The reaction mixture was heated at reflux for 2 hours and the solvent was removed in vacuo to give 7-chloro-2-(2-isopropenyl-3-methyl-3H-imidazol-4-yl)-thieno[3,2-b]pyridine (145 mg, 99%). MS 290, 292 (MH+), HPLC Rf: 5.29 min; HPLC purity 99%.

B. The title compound (121 mg, 60%) was prepared from 7-chloro-2-(2-isopropenyl-3-methyl-3H-imidazol-4-yl)-thieno[3,2-b]pyridine and 2-methyl-1H-indol-5-ylamine by a procedure analogous to Example 1. MS: 400 (MH+); HPLC Rf: 4.93 min; HPLC purity: 98%.

### EXAMPLE 7

### [2-(2-Aminomethyl-3-methyl-3H-imidazol-4-yl)-thieno[3,2-b]pyridin-7-yl]-(2-methyl-1H-indol-5-yl)-amine

1-Methyl-5-[7-(2-methyl-1H-indol-5-ylamino)-thieno[3,2-b]pyridin-2-yl]-1H-imidazole-2-carbonitrile (192 mg, 0.50 mmol) was dissolved in anhydrous THF and triethyl amine (75 uL, 0.75 mmol) was added. The solution was stirred at room temperature for 20 minutes and became light red. Lithium aluminum hydride solution (1.0 M in ether, 2.5 mL, 2.5 mmol) was added dropwise. The reaction mixture was stirred at room temperature for 30 minutes then quenched with ethyl acetate. The reaction mixture was partitioned between ethyl acetate and 1M HCl, and the organic layer was extracted with an additional portion of 1M HCl. The combined aqueous layers were treated with saturated sodium bicarbonate solution to an ultimate pH of 8. The resulting aqueous solution was extracted with ethyl acetate. The combined organic phases were washed with brine and dried over sodium sulfate. Evaporation of solvent in vacuo gave the title compound as a yellow solid (136 mg, 70%). MS: 389 (MH+); HPLC Rf: 3.16 min; HPLC purity: 95%.

### EXAMPLE 8

### 1-Methyl-5-[7-(2-methyl-1H-indol-5-ylamino)-thieno[3,2-b]pyridin-2-yl]-1H-imidazole-2-carboxylic acid [3-(4-methyl-piperazin-1-yl)-propyl]-amide

A. 5-(7-Chloro-thieno[3,2-b]pyridin-2-yl)-1-methyl-1H-imidazole-2-carboxylic acid [3-(4-methyl-piperazin-1-yl)-propyl]-amide was prepared from 5-(7-chloro-thieno[3,2-b]pyridin-2-yl)-1-methyl-1H-imidazole-2-carboxylic acid methyl ester and 1-(3-aminopropyl)-4-methylpiperazine by a procedure analogous to Example 3. MS: 433, 435 (MH+), HPLC Rf: 4.21 min; HPLC purity 99%.

B. The title compound was prepared from 5-(7-chloro-thieno[3,2-b]pyridin-2-yl)-1-methyl-1H-imidazole-2-carboxylic acid [3-(4-methyl-piperazin-1-yl)-propyl]-amide and 2-methyl-1H-indol-5-ylamine by a procedure analogous to Example 3. MS: 543 (MH+); HPLC Rf: 3.72 min; HPLC purity: 99%.

### EXAMPLE 9

### 1-Methyl-5-[7-(2-methyl-1H-indol-5-ylamino)-thieno[3,2-b]pyridin-2-yl]-1H-imidazole-2-carboxylic acid (pyridin-2-ylmethyl)-amide

A. 5-(7-Chloro-thieno[3,2-b]pyridin-2-yl)-1-methyl-1H-imidazole-2-carboxylic acid (pyridin-2-ylmethyl)-amide was prepared from 5-(7-chloro-thieno[3,2-b]pyridin-2-yl)-1-methyl-1H-imidazole-2-carboxylic acid methyl ester and 2-aminomethylpyridine by a procedure analogous to Example 3. MS 384, 386 (MH+); HPLC Rf: 5.24 min; HPLC purity 98%.

B. The title compound was prepared from 5-(7-chloro-thieno[3,2-b]pyridin-2-yl)-1-methyl-1H-imidazole-2-carboxylic acid (pyridin-2-ylmethyl)-amide and 2-methyl-1H-indol-5-ylamine by a procedure analogous to Example 3. MS: 494 (MH+); HPLC Rf: 4.72 min; HPLC purity: 95%.

### EXAMPLE 10

### 1-Methyl-5-[7-(2-methyl-1H-indol-5-ylamino)-thieno[3,2-b]pyridin-2-yl]-1H-imidazole-2-carboxylic Acid (2-morpholin-4-yl-ethyl)-amide

A. 5-(7-Chloro-thieno[3,2-b]pyridin-2-yl)-1-methyl-1H-imidazole-2-carboxylic acid (2-morpholin-4-yl-ethyl)-amide was prepared from 5-(7-chloro-thieno[3,2-b]pyridin-2-yl)-1-methyl-1H-imidazole-2-carboxylic acid methyl ester and 2-(4-morpholino)ethylamine by a procedure analogous to Example 3. MS: 406, 408 (MH+), HPLC Rf 3.15 min; HPLC purity 95%.

B. The title compound was prepared from 5-(7-chloro-thieno[3,2-b]pyridin-2-yl)-1-methyl-1H-imidazole-2-carboxylic acid (2-morpholin-4-yl-ethyl)-amide and 2-methyl-1H-indol-5-ylamine by a procedure analogous to Example 3. MS: 516 (MH+); HPLC Rf: 3.84 min; HPLC purity: 99 %.

### EXAMPLE 11

### 1-Methyl-5-[7-(2-methyl-1H-indol-5-ylamino)-thieno[3,2-b]pyridin-2-yl]-1H-imidazole-2-carboxylic Acid (2-pyridin-4-yl-ethyl)-amide

A. 5-(7-Chloro-thieno[3,2-b]pyridin-2-yl)-1-methyl-1H-imidazole-2-carboxylic acid (2-pyridin-4-yl-ethyl)-amide was prepared from 5-(7-chloro-thieno[3,2-b]pyridin-2-yl)-1-methyl-1H-imidazole-2-carboxylic acid methyl ester and 2-(4-pyridynyl)ethylamine by a procedure analogous to Example 3. MS: 398, 400 (MH+), HPLC Rf: 4.98 min; HPLC purity: 98%.

B. The title compound was prepared from 5-(7-chloro-thieno[3,2-b]pyridin-2-yl)-1-methyl-1H-imidazole-2-carboxylic acid (2-pyridin-4-yl-ethyl)-amide and 2-methyl-1H-indol-5-ylamine by a procedure analogous to Example 3. MS: 508 (MH+); HPLC Rf: 4.68 min; HPLC purity: 99%.

### EXAMPLE 12

### 1-Methyl-5-[7-(2-methyl-1H-indol-5-ylamino)-thieno[3,2-b]pyridin-2-yl]-l H-imidazole-2-carboxylic Acid (2-piperidin-1-yl-ethyl)-amide

A. 5-(7-Chloro-thieno[3,2-b]pyridin-2-yl)-1-methyl-1H-imidazole-2-carboxylic acid (2-piperidin-1-yl-ethyl)-amide was prepared from 5-(7-chloro-thieno[3,2-b]pyridin-2-yl)-1-methyl-1H-imidazole-2-carboxylic acid methyl ester and 2-(1-piperidinyl)ethylamine by a procedure analogous to Example 3. MS: 404, 406 (MH+); HPLC Rf: 4.98 min; HPLC purity: 95%.

B. The title compound was prepared from 5-(7-chloro-thieno[3,2-b]pyridin-2-yl)-1-methyl-1H-imidazole-2-carboxylic acid (2-piperidin-1-yl-ethyl)-amide and 2-methyl-1H-indol-5-ylamine by a procedure analogous to Example 3. MS: 514 (MH+); HPLC Rf: 4.01 min; HPLC purity: 97%.

### EXAMPLE 13

### 1-Methyl-5-[7-(2-methyl-1H-indol-5-ylamino)-thieno[3,2-b]pyridin-2-yl]-1H-imidazole-2-carboxylic Acid pyridin-2-ylamide

A. 5-(7-Chloro-thieno[3,2-b]pyridin-2-yl)-1-methyl-1H-imidazole-2-carboxylic acid pyridin-2-ylamide was prepared from 5-(7-chloro-thieno[3,2-b]pyridin-2-yl)-1-methyl-1H-imidazole-2-carboxylic acid methyl ester and 2-aminopyridine by a procedure analogous to Example 3. MS: 370, 372 (MH+); HPLC Rf: 6.38 min; HPLC purity 97%.

B. The title compound was prepared from 5-(7-chloro-thieno[3,2-b]pyridin-2-yl)-1-methyl-1H-imidazole-2-carboxylic acid pyridin-2-ylamide and 2-methyl-1H-indol-5-ylamine by a procedure analogous to Example 3. MS: 480 (MH+); HPLC Rf: 5.62 min; HPLC purity: 92%.

### EXAMPLE 14

### 1-Methyl-5-[7-(2-methyl-1H-indol-5-ylamino)-thieno[3,2-b]pyridin-2-yl]-1H-imidazole-2-carboxylic Acid (3-morpholin-4-yl-propyl)-amide

A. 5-(7-Chloro-thieno[3,2-b]pyridin-2-yl)-1-methyl-1H-imidazole-2-carboxylic acid (3-morpholin-4-yl-propyl)-amide was prepared from 5-(7-chloro-thieno[3,2-b]pyridin-2-yl)-1-methyl-1H-imidazole-2-carboxylic acid methyl ester and 3-(4-morpholino)propylamine by a procedure analogous to Example 3. MS: 420, 422 (MH+); HPLC Rf 4.24 min; HPLC purity 98%.

B. The title compound was prepared from 5-(7-chloro-thieno[3,2-b]pyridin-2-yl)-1-methyl-1H-imidazole-2-carboxylic acid (3-morpholin-4-yl-propyl)-amide and 2-methyl-1H-indol-5-ylamine by a procedure analogous to Example 3. MS: 530 (MH+); HPLC Rf: 3.33 min; HPLC purity: 92%.

### EXAMPLE 15

### 1-Methyl-5-[7-(2-methyl-1H-indol-5-ylamino)-thieno[3,2-b]pyridin-2-yl]-1H-imidazole-2-carboxylic Acid Dimethylamide

A. 5-(7-Chloro-thieno[3,2-b]pyridin-2-yl)-1-methyl-1H-imidazole-2-carboxylic acid dimethylamide was prepared from 5-(7-chloro-thieno[3,2-b]pyridin-2-yl)-1-methyl-1H-imidazole-2-carboxylic acid methyl ester and dimethylamine by a method analogous to Example 3. MS: 321, 323 (MH+), HPLC Rf 4.37 min; HPLC purity 98%.

B. The title compound was prepared from 5-(7-Chloro-thieno[3,2-b]pyridin-2-yl)-1-methyl-1H-imidazole-2-carboxylic acid dimethylamide and 2-methyl-1H-indol-5-ylamine by a procedure analogous to Example 3. MS: 431 (MH+); HPLC Rf: 4.05 min; HPLC purity: 95%.

### EXAMPLE 16

### 1-Methyl-5-[7-(2-methyl-1H-indol-5-ylamino)-thieno[3,2-b]pyridin-2-yl]-1H-imidazole-2-carboxylic Acid Methylamide

A. 5-(7-Chloro-thieno[3,2-b]pyridin-2-yl)-1-methyl-1H-imidazole-2-carboxylic acid methylamide was prepared from 5-(7-chloro-thieno[3,2-b]pyridin-2-yl)-1-methyl-1H-imidazole-2-carboxylic acid methyl ester and methylamine by a method analogous to Example 3. MS: 307, 309 (MH+); HPLC Rf: 4.732 min; HPLC purity 98%

B. The title compound was prepared from 5-(7-chloro-thieno[3,2-b]pyridin-2-yl)-1-methyl-1H-imidazole-2-carboxylic acid methylamide and 2-methyl-1H-indol-5-ylamine by a procedure analogous to Example 3. MS: 417 (MH+); HPLC Rf: 4.35 min; HPLC purity: 99%.

### EXAMPLE 17

### 1-Methyl-5-[7-(2-methyl-1H-indol-5-ylamino)-thieno[3,2-b]pyridin-2-yl]-1H-imidazole-2-carboxylic Acid (2-pyridin-2-yl-ethyl)-amide

A. 5-(7-Chloro-thieno[3,2-b]pyridin-2-yl)-1-methyl-1H-imidazole-2-carboxylic acid (2-pyridin-2-yl-ethyl)-amide was prepared from 5-(7-chloro-thieno[3,2-b]pyridin-2-yl)-1-methyl-1H-imidazole-2-carboxylic acid methyl ester and 2-(2-pyridinyl)ethylamine by a method analogous to Example 3. MS: 398, 400 (MH+); HPLC Rf: 5.23;HPLC purity 95%

B. The title compound was prepared from 5-(7-Chloro-thieno[3,2-b]pyridin-2-yl)-1-methyl-1H-imidazole-2-carboxylic acid (2-pyridin-2-yl-ethyl)-amide and 2-methyl-1H-indol-5-ylamine by a procedure analogous to Example 3. MS: 508 (MH+); HPLC Rf: 4.73 min; HPLC purity: 98%.

### EXAMPLE 18

### 2-{1-Methyl-5-[7-(2-methyl-1H-indol-5-ylamino)-thieno[3,2-b]pyridin-2-yl]-1H-imidazol-2-yl}-propane-1,2-diol

A. A solution of 7-chloro-2-(3-methyl-3H-imidazol-4-yl)-thieno[3,2-b]pyridine (250 mg, 1.0 mmol) in anhydrous THF (30 mL) was cooled to −78° C. via dry ice/acetone bath. n-BuLi (2.5 M in hexanes, 440 uL, 1.1 mmol) was added slowly, and the solution was stirred at −78° C. for 30 minutes. 1-(tert-butyldimethylsilyloxy)-2-propane (376 mg, 2.0 mmol) was added dropwise. The resulting heterogeneous reaction mixture was stirred at −78° C. for 30 minutes and the dry ice/acetone bath was removed. After warming to room temperature, the reaction mixture was diluted with methanol. The crude material was concentrated onto 1 gram of silica gel powder by removing the solvent in vacuo, and the residue was purified by flash chromatography eluting with dichloromethane/methanol (100/3 v/v) to give 2-[5-(7-chloro-thieno[3,2-b]pyridin-2-yl)-1-methyl-1H-imidazol-2-yl]-propane-1,2-diol as a white solid (220 mg, 0.50 mmol, 50%). MS: 439, 451; HPLC Rf: 7.64 min; HPLC purity 93%.

B. The title compound (152 mg, 70%) was prepared from 2-[5-(7-chloro-thieno[3,2-b]pyridin-2-yl)-1-methyl-1H-imidazol-2-yl]-propane-1,2-diol and 2-methyl-1H-indol-5-ylamine by a procedure analogous to Example 1. MS: 434 (MH+); HPLC Rf: 3.75 min; HPLC purity: 98%.

### EXAMPLE 19

### 1-Methyl-5-[7-(2-methyl-1H-indol-5-ylamino)-thieno[3,2-b]pyridin-2-yl]-1H-imidazole-2-carboxylic Acid Amide

A. 5-(7-Chloro-thieno[3,2-b]pyridin-2-yl)-1-methyl-1H-imidazole-2-carboxylic acid amide was prepared from 5-(7-chloro-thieno[3,2-b]pyridin-2-yl)-1-methyl-1H-imidazole-2-carboxylic acid methyl ester and ammonia by a method analogous to Example 3. MS: 293, 295 (MH+), HPLC Rf: 4.38; HPLC purity 98%

B. The title compound was prepared from 5-(7-chloro-thieno[3,2-b]pyridin-2-yl)-1-methyl-1H-imidazole-2-carboxylic acid methylamide and 2-methyl-1H-indol-5-ylamine by a procedure analogous to Example 3. MS: 403 (MH+); HPLC Rf: 3.98 min; HPLC purity: 95%.

### EXAMPLE 20

### 1-Methyl-5-[7-(2-methyl-1H-indol-5-ylamino)-thieno[3,2-b]pyridin-2-yl]-1H-imidazole-2-carboxylic Acid (pyridin-4-ylmethyl)-amide

A. 5-(7-Chloro-thieno[3,2-b]pyridin-2-yl)-1-methyl-1H-imidazole-2-carboxylic acid pyridin-4-ylmethylamide was prepared from 5-(7-chloro-thieno[3,2-b]pyridin-2-yl)-1-methyl-1H-imidazole-2-carboxylic acid methyl ester and 4-aminomethyl pyridine by a method analogous to Example 3. MS: 384, 386 (MH+); HPLC Rf: 4.95 min; HPLC purity 95%.

B. The title compound was prepared from 5-(7-chloro-thieno[3,2-b]pyridin-2-yl)-1-methyl-1H-imidazole-2-carboxylic acid pyridin-4-ylmethylamide and 2-methyl-1H-indol-5-ylamine by a procedure analogous to Example 3. MS: 494 (MH+); HPLC Rf: 4.44 min; HPLC purity: 85%.

### EXAMPLE 21

### 1-Methyl-5-[7-(2-methyl-1H-indol-5-ylamino)-thieno[3,2-b]pyridin-2-yl]-1H-imidazole-2-carboxylic Acid Ethylamide

A. 5-(7-Chloro-thieno[3,2-b]pyridin-2-yl)-1-methyl-1H-imidazole-2-carboxylic acid ethylamide was prepared from 5-(7-chloro-thieno[3,2-b]pyridin-2-yl)-1-methyl-1H-imidazole-2-carboxylic acid methyl ester and ethylamine by a method analogous to Example 3. MS: 321, 323 (MH+); HPLC Rf: 5.25 min; HPLC purity 85%.

B. The title compound was prepared from 5-(7-chloro-thieno[3,2-b]pyridin-2-yl)-1-methyl-1H-imidazole-2-carboxylic acid ethylamide and 2-methyl-1H-indol-5-ylamine by a procedure analogous to Example 3. MS: 431 (MH+); HPLC Rf: 4.64 min; HPLC purity: 95%.

### EXAMPLE 22

### 1-Methyl-5-[7-(2-methyl-1H-indol-5-ylamino)-thieno[3,2-b]pyridin-2-yl]-1H-imidazole-2-carboxylic Acid Methyl-pyridin-3-ylmethyl-amide

A. 5-(7-Chloro-thieno[3,2-b]pyridin-2-yl)-1-methyl-1H-imidazole-2-carboxylic acid methyl-pyridin-3-ylmethyl-amide was prepared from 5-(7-chloro-thieno[3,2-b]pyridin-2-yl)-1-methyl-1H-imidazole-2-carboxylic acid methyl ester and 3-methylaminomethyl pyridine by a method analogous to Example 3. MS: 398, 400 (MH+); HPLC Rf: 4.80 min; HPLC purity 90%.

B. The title compound was prepared from 5-(7-chloro-thieno[3,2-b]pyridin-2-yl)-1-methyl-1H-imidazole-2-carboxylic acid methyl-pyridin-4-ylmethyl-amide and 2-methyl-1H-indol-5-ylamine by a procedure analogous to Example 3. MS: 508 (MH+); HPLC Rf: 4.45 min; HPLC purity: 85%.

### EXAMPLE 23

### 1-Methyl-5-[7-(2-methyl-1H-indol-5-ylamino)-thieno[3,2-b]pyridin-2-yl]-1H-imidazole-2-carboxylic Acid Diethylamide

A. 5-(7-Chloro-thieno[3,2-b]pyridin-2-yl)-1-methyl-1H-imidazole-2-carboxylic acid diethylamide was prepared from 5-(7-chloro-thieno[3,2-b]pyridin-2-yl)-1-methyl-1H-imidazole-2-carboxylic acid methyl ester and diethylamine by a method analogous to Example 3. MS: 349, 351 (MH+), HPLC Rf: 5.27; HPLC purity: 85%.

B. The title compound was prepared from 5-(7-chloro-thieno[3,2-b]pyridin-2-yl)-1-methyl-1H-imidazole-2-carboxylic acid diethylamide and 2-methyl-1H-indol-5-ylamine by a procedure analogous to Example 3. MS: 459 (MH+); HPLC Rf: 4.80 min; HPLC purity: 90%.

### EXAMPLE 24

### 1-Methyl-5-[7-(2-methyl-1H-indol-5-ylamino)-thieno[3,2-b]pyridin-2-yl]-1H-imidazole-2-carboxylic Acid Ethyl-methyl-amide

A. 5-(7-Chloro-thieno[3,2-b]pyridin-2-yl)-1-methyl-1H-imidazole-2-carboxylic acid ethyl-methylamide was prepared from 5-(7-chloro-thieno[3,2-b]pyridin-2-yl)-1-methyl-1H-imidazole-2-carboxylic acid methyl ester and N-methylethylamine by a method analogous to Example 3. MS: 335, 337 (MH+); HPLC Rf: 4.81 min; HPLC purity: 95%.

B. The title compound was prepared from 5-(7-chloro-thieno[3,2-b]pyridin-2-yl)-1-methyl-1H-imidazole-2-carboxylic acid ethyl-methylamide and 2-methyl-1H-indol-5-ylamine by a procedure analogous to Example 3. MS: 445 (MH+); HPLC Rf: 4.42 min; HPLC purity: 95%.

### EXAMPLE 25

### (2-methyl-1H-indol-5-yl)-[2-(5-methyl-[1,3,4]oxadiazol-2-yl)-thieno[3,2-b]pyridin-7-yl]-amine

A. 60.00 g (355 mmol) 7-chloro-thieno[3,2-b]pyridine was suspended in one liter of dry THF, and the reaction mixture was cooled to −78° C. To this was added 156 mL (39.0 mmol) of 2.5M n-buLi solution (in hexanes). The reaction mixture was allowed to stir at −78° C. for two hours. Gaseous CO₂ was bubbled through the reaction solution for twenty-five minutes. The solvent was removed under reduced pressure, and the off-white solid was suspended in ethyl ether and filtered to obtain lithium 7-chloro-thieno[3,2-b]pyridine-2-carboxylate in a 98% yield as an off-white solid.

C₈H₃ClLiNO₂S: ¹H NMR (d₆-DMSO): 8.60 (d, 1H, J=5.2 Hz), 7.66 (S, 1 H), 7.53 (d, 1 H, J=5.2 Hz) ppm.

B. 60.0 g (282 mmol) Lithium 7-chloro-thieno[3,2-b]pyridine-2-carboxylate was taken into a mixture of 600 mL dry dichloromethane and 6.00 mL dimethyl formamide(DMF). To this was added 30.0 mL (423 mmol) of thionyl chloride. The reaction mixture was heated to reflux for six hours. The reaction mixture was then allowed to cool to room temperature, and volatiles were removed under reduced pressure followed by the removal of excess thionyl chloride with as a toluene azeotrope to afford 7-chloro-thieno[3,2-b]pyridine-2-carbonyl chloride in 97% yield as a brown solid.

C₈H₃Cl₂NOS: ¹H NMR (CD₃OD): 9.12 (d, 1 H, J=6.2 Hz), 8.41 (s, 1 H), 8.25 (d, 1 H, J=6.2 Hz) ppm.

C. 2.50 g (10.5 mmol) 7-Chloro-thieno[3,2-b]pyridine-2-carbonyl chloride was taken into 30.0 mL dry dichloromethane. 1.55 g (20.9 mmol) acetic hydrazide was added portion-wise as a solid. The reaction mixture was allowed to stir at room temperature under nitrogen. After three hours, the reaction mixture had turned to a slurry. The solvents were removed under reduced pressure to give an off-white solid which was purified by flash chromatography (99:1:0.1 tricholormethane (“CHCl₃”):methanol (“CH₃OH”):ammonium hydroxide (“NH₄OH”)) to give 7-chloro-thieno[3,2-b]pyridine-2-carboxylic acid N′-acetyl-hydrazide in 85% yield as an off-white solid.

C₁₀H₈ClN₃O₂S: APCI m/z: 269.9/271.9 (MH+); 267.9/269.9 (MH−). ¹H NMR (d₆-DMSO): 10.84 (s, 1 H), 10.07 (s, 1 H), 8.72 (d, 1 H, J=5.0 Hz), 8.35 (s, 1 H), 7.70 (d, 1 H, J=5.0 Hz), 1.92 (s, 3 H) ppm.

D. 1.00 g (3.71 mmol) 7-Chloro-thieno[3,2-b]pyridine-2-carboxylic acid N′-acetyl-hydrazide was taken into 66.0 mL thionyl chloride with 6.60 mL DMF. The reaction mixture was heated to 85° C. for two hours at which time the reaction mixture had attained complete solution and turned to a slightly yellow color. The thionyl chloride was removed under reduced pressure via a toluene azeotrope to give an off-white solid that was purified over silica gel (98:2:0.20 CHCl₃:CH₃OH:NH₄OH) to obtain 7-chloro-2-(5-methyl-[1,3,4]oxadiazol-2-yl)-thieno[3,2-b]pyridine in 58% yield as an off-white solid.

C₁₀H₆ClN₃OS: APCI m/z: 252.0/254.0 (MH+); ¹H NMR (d₆-DMSO): 8.75 (d, 1H, J=5.2 Hz), 8.28 (s, 1 H), 7.73 (d, 1 H, J=5.2 Hz), 2.47 (s, 3 H) ppm.

E. 250 mg (0.993 mmol) 7-Chloro-2-(5-methyl-[1,3,4]oxadiazol-2-yl)-thieno[3,2-b]pyridine was taken into 2.00 mL ethanol and 400 uL dichloroethane with 145 mg (0.993 mmol) 2-methyl-1H-indol-5-yl amine. The reaction mixture was heated to 90° C. overnight. The reaction mixture was allowed to cool to room temperature and loaded directly onto silica gel through evaporation. The title compound was obtained in pure form through column chromatography (98:2:0.20 CHCl₃:CH₃OH:NH₄OH) in a 16% yield as a bright yellow solid.

C₁₉H₁₅N₅OS: APCI m/z: 362.0 (MH+); ¹H NMR (d₆-DMSO): 8.86 (s, 1 H), 8.22 (d, 1 H, J=5.4 Hz), 7.90 (s, 1 H), 7.64 (d, 1 H, J=8.70 Hz), 7.28 through 7.26 (comp., 2 H), 6.90 (d, 1 H, J=8.70 Hz), 6.63 (d, 1 H, 5.4 Hz), 6.10 (s, 1 H), 2.55 (s, 3 H), 2.36 (s, 3 H) ppm.

### EXAMPLE 26

### 2-[7-(2-Methyl-1H-indol-5-ylamino)-thieno[3,2-b]pyridin-2-yl-thiazole-4-carboxylic Acid Ethyl Ester

A. 60.00 g (360 mmol) 7-chloro-thieno[3,2-b]pyridine was taken into 600 mL dry THF. The reaction solution was degassed for ten minutes before cooling to −78° C. 170 mL (432 mmol) n-buLi was added drop-wise at such a rate that the temperature never exceeded −65° C. The reaction solution turned dark and then developed a yellow precipitate. The reaction mixture was allowed to stir at −78° C. for three hours. 43.2 mL (468 mmol) methoxymethyl isothiocyanate in 400 mL THF was slowly added. The reaction mixture turned dark after the addition was complete. The reaction mixture was allowed to stir at −78° C. for three hours then was warmed to room temperature and quenched with 150 mL saturated ammonium chloride (NH₄Cl) solution, resulting in a bright yellow color. The THF was removed under reduced pressure, and 7-chloro-thieno[3,2-b]pyridine-2-carbothioic acid methoxymethyl-amide was obtained through filtration in 95% yield as a yellow-orange solid.

C₁₀H₉ClN₂OS₂: APCI m/z: 273.0/274.9 (MH+); ¹H NMR (d₆-DMSO): 8.60 (d, 1H, J=5.0 Hz), 8.13 (s, 1 H), 7.55 (d, 1 H, J=5.0 Hz), 4.97 (s, 2 H), 2.46 (s, 3 H).

B. 93.86 g (344 mmol) 7-Chloro-thieno[3,2-b]pyridine-2-carbothioic acid methoxymethyl-amide was taken into 900 mL THF. To this was slowly added 344 mL (344 mmol) 1 N HCl (aq). The reaction was heated at reflux for three days. The reaction mixture was allowed to cool to 0° C. and quenched with 300 mL concentrated NH₄OH, and the THF was removed under reduced pressure. The solid yellow residue was triturated with ethyl acetate and 7-chloro-thieno[3,2-b]pyridine-2-carbothioic acid amide was obtained through filtration in an 85% yield.

C₈H₅ClN₂S₂: APCI m/z: 228.9/230.9 (MH+); ¹H NMR (d₆-DMSO): 10.24 (s, 1 H), 9.94 (s, 1 H), 8.65 (d, 1 H, J=5.0 Hz), 8.19 (s, 1 H), 7.62 (d, 1 H, J=5.0 Hz) ppm.

C. 5.00 g (21.9 mmol) 7-Chloro-thieno[3,2-b]pyridine-2-carbothioic acid amide was suspended in 60.0 mL THF with 4.11 mL (32.8 mmol) ethyl bromo pyruvate. The reaction mixture was allowed to stir at room temperature under nitrogen overnight. The reaction mixture was then cooled to 0° C., and 30 mL trifluoroacetic anhydride was added. Complete solution of the reaction mixture was accompanied by a dark color change. The reaction mixture was allowed to stir at room temperature for four hours before cooling to 0° C. The reaction was quenched with 30 mL concentrated NH₄OH, and the THF was removed under reduced pressure. The dark, oily residue was partitioned between H₂O and ethyl acetate. Aqueous work-up afforded a brown oil. 2-(7-chloro-thieno[3,2-b]pyridin-2-yl)-thiazole-4-carboxylic acid ethyl ester was obtained in 67% yield through column chromatography (99.5:0.5:0.05 CHCl₃:CH₃OH:NH₄OH) as a pale yellow solid.

C₁₃H₉ClN₂O₂S₂: APCI m/z: 325.0/327.0 (MH+), 232.9/325.9 (MH−); ¹H NMR (d₆-DMSO): 8.67 (d, 1 H, J=5.0 Hz), 8.37 (s, 1 H), 7.62 (d, 1 H, J=5.0 Hz), 3.98 (q, 2H, J=7.0 Hz), 1.13 (t, 3 H, J=7.0 Hz) ppm.

D. The title compound was obtained in a 19% yield as a bright yellow solid by a procedure analogous to Example 25 using 2-(7-chloro-thieno[3,2-b]pyridin-2-yl)-thiazole-4-carboxylic acid ethyl ester as the substrate.

C₂₂H₁₈N₄O₂S₂: APCI m/z: 435.0 (MH+); ¹H NMR (CD₃OD): 8.50 (s, 1 H), 8.19 (d, 1 H, J=6.2 Hz), 7.92 (s, 1 H), 7.43 (s, 1 H), 7.38 (d, 1 H, J=8.4 Hz), 7.03 (d, 1 H, J=6.2 Hz), 6.20 (s, 1 H), 4.43 (q, 2 H, J=7.1 Hz), 2.47 (s, 3 H), 1.43 (t, 3 H, J=7.1 Hz) ppm.

### EXAMPLE 27

### {2-[7-(2-Methyl-1H-indol-5-amino)-thieno[3,2-b]pyridin-2-yl]-thiazol-4-yl}-acetic Acid Ethyl Ester

A. 100 mg (0.437 mmol) 7-Chloro-thieno[3,2-b]pyridine-2-carbothioic acid amide was taken into 500 mL ethanol with 108 mg (0.656 mmol) 4-chloro ethyl acetoacetate. The reaction mixture was heated to reflux for two days. The resulting yellow solution was loaded onto silica gel under reduced pressure and purified though column chromatography (99.5:0.5:0.05 CHCl₃:CH₃OH:NH₄OH) to obtain 2-(7-chloro-thieno[3,2-b]pyridin-2-yl)-thiazol-4-yl]-acetic acid ethyl ester as a yellow solid in 57% yield.

C₁₄H₁₁ClN₂O₂S₂: APCI m/z: 339.1/341.1 (MH+); ¹H NMR (CD₃OD): 8.58 (d, 1 H, J=5.0 Hz), 7.96 (s, 1 H), 7.56 (s, 1 H), 7.48 (d, 1 H, J=5.0 Hz), 4.19 (q, 2 H, J=7.1 Hz), 3.89 (s, 2 H), 1.27 (t, 3 H, J=7.1 Hz) ppm.

B. The title compound was obtained in a 25% yield as a bright yellow solid using a method analogous to Example 25 using 2-(7-chloro-thieno[3,2-b]pyridin-2-yl)-thiazol-4-yl]-acetic acid ethyl ester as a substrate.

C₂₃H₂₀H₄O₂S₂: APCI m/z: 450.2 (MH+); ¹H NMR (CD₃OD): 8.12 (d, 1 H, J=5.8), 7.76 (s, 1 H), 7.47 (s, 1 H), 7.30 (d, 1 H, J=8.7 Hz), 6.97 (d, 1 H, J=8.7 Hz), 6.64 (d, 1 H, J=5.4Hz), 6.13 (s, 1 H), 4.17 (q, 2 H, J=7.1 Hz), 3.85 (s, 2 H), 2.42 (s, 3 H), 1.25 (t, 3 H, J=7.1 Hz) ppm.

### EXAMPLE 28

### 4-Methyl-2-[7-(2-methyl-1H-indol-5-ylamino)-thieno[3,2-b]pyridin-2-yl]-thiazole-5-carboxylic Acid Ethyl Ester

A. 2-(7-Chloro-thieno[3,2-b]pyridin-2-yl)-4-methyl-thiazole-5-carboxylic acid ethyl ester was prepared in 36% yield as a yellow solid by a procedure analogous to Example 28 using ethyl 2-chloroacetoacetate as the coupling reagent.

C₁₄H₁₁ClN₂O₂S₂: APCI m/z 339.1/341.0 (MH+); ¹H NMR (CDCl₃): 8.68 (d, 1 H , J=5.4 Hz), 8.24 (s, 1 H), 7.51 (d, 1 H, J=5.4 Hz), 4.36 (q, 2 H, J=7.1 Hz), 2.77 (s, 3 H), 1.38 (t, 3 H, J=7.1 Hz) ppm.

B. The title compound was made in 14% yield as a bright yellow solid through a protocol similar to that of Example 25 using 2-(7-chloro-thieno[3,2-b]pyridin-2-yl)-4-methyl-thiazole-5-carboxylic acid ethyl ester as the substrate.

C₂₃H₂₀H₄O₂S₂: APCI m/z: 448.9 (MH+); ¹H NMR (CD₃OD): 8.19 (d, 1 H, J=7.1 Hz), 7.91 (s, 1 H), 7.44 (s, 1 H), 7.40 (d, 1 H, J=8.7 Hz), 7.02 (d, 1 H, J=8.7 Hz), 6.83 (d, 1 H, J=7.1 Hz), 6.21 (s, 1 H), 4.35 (q, 2 H, J=7.1 Hz), 2.69 (s, 3 H), 2.45 (s, 3 H), 1.36 (t, 3 H, J=7.1 Hz) ppm.

### EXAMPLE 29

### {4-Methyl-2-[7-(2-methyl-1H-indol-5-ylamino)-thieno[3,2-b]pyridin-2-yl]-thiazol-5-yl}-methanol

A. 100 mg (0.233 mmol) 4-Methyl-2-[7-(2-methyl-1H-indol-5-ylamino)-thieno[3,2-b]pyridin-2-yl]-thiazole-5-carboxylic acid ethyl ester was taken into 1.00 mL THF and cooled to 0° C. To this was added 379 uL (0.379 mmol) lithium aluminum hydride (LAH) solution (1.0 M in THF). The reaction mixture was removed from the ice bath and allowed to stir at room temperature for one hour. The reaction mixture was again cooled to 0° C. and 1.00 mL H₂O was added to quench the reaction mixture. Ethyl acetate and saturated sodium bicarbonate were added. Aqueous work-up gave a yellow solid. This solid was purified over silica gel (98:2:0.20 CHCl₃:CH₃OH:NH₄OH) to obtain the title compound in an 11% yield as a yellow solid.

C₂₁H₁₈N₄OS₂: APCI m/z 407.2 (MH+); ¹H NMR (CD₃OD): 8.10 (d, 1 H, J=5.4 Hz), 7.69 (s, 1 H), 7.34 (s, 1 H), 7.29 (d, 1 H, J=8.3), 6.96 (d, 1 H, J=8.3), 6.61 (d, 1 H, J=5.4 Hz), 6.12 (s, 1 H), 4.87 (s, 2 H), 2.42 (s, 3 H), 2.37 (s, 3 H) ppm.

### EXAMPLE 30

### 2-{2-[7-(2-Methyl-1H-indol-5-ylamino)-thieno[3,2-b]pyridin-2-yl]-thiazol-4-yl}-propan-2-ol

A. 220 mg (0.677 mmol) 2-(7-Chloro-thieno[3,2-b]pyridin-2-yl)-thiazole-4-carboxylic acid ethyl ester was taken into 3.0 mL dry THF and cooled to −78° C. To this was added 564 uL (1.69 mmol) methyl magnesium bromide solution (3.0 M in THF) dropwise. The reaction mixture was allowed to stir at −78° C. for four hours then was removed from the cooling bath and quenched with 1.00 mL saturated NH₄Cl solution. Ethyl acetate and saturated sodium hydrogen carbonate (“NaHCO₃”) were added. Aqueous work-up gave a brown solid that yielded 2-[2-(7-chloro-thieno[3,2-b]pyridin-2-yl)-thiazol-4-yl]-propan-2-ol in 48% yield after chromatography (98:2:0.20 CHCl₃:CH₃OH:NH₄OH) as a yellow solid.

C₁₃H₁₁ClN₂OS₂: APCI m/z 311.1/313.1 (MH+).

B. The title compound was obtained in a procedure similar to that Example 25 using 2-[2-(7-chloro-thieno[3,2-b]pyridin-2-yl)-thiazol-4-yl]-propan-2-ol as the substrate in a 17% yield as a bright yellow solid.

C₂₂H₂₀H₄OS₂: APCI m/z: 421.3 (MH+); ¹H NMR (CD₃OD): 8.11 (d, 1 H, J=5.4 Hz), 7.74 (s, 1 H), 7.41 (s, 1 H). 7.35 (s, 1 H), 7.30 (d, 1 H, J=8.3 Hz), 6.97 (d, 1 H, J=8.3 Hz), 6.62 (d, 1 H, J=5.4 Hz), 6.13 (s, 1 H), 2.42 (s, 3 H), 1.58 (s, 6 H).

### EXAMPLE 31

### (2-methyl-1H-indol-5-yl)-(2-4 pyrrolidin-1-ylmethyl-thiazole-2-yl)-thieno[3,2-b]pyridin-7-yl)-amine

A. 2-(7-Chloro-thieno[3,2-b] pyridine-2-yl)-thiazol-4-yl-methanol was made from 2-(7-chloro-thieno[3,2-b]pyridin-2-yl)-thiazole-4-carboxylic acid ethyl ester in a manner similar to Example 29. C₁₁H₇ClN₂OS₂: APCI m/z: 283.0/284.9 (MH+), HPLC Rf: 5.03 min.

B. 7-Chloro-2-(4-chloromethyl-thiazole-2-yl)-thieno[3,2-b] pyridine was made from 2-(7-chloro-thieno[3,2-b] pyridine-2-yl)-thiazol-4-yl-methanol in a manner similar to Example 25. C₁₁H₆Cl₂N₂S₂: APCI m/z: 300.9/302.9/304.9 (MH+); ¹H NMR (CDCl₃): 8.59 (d, 1H), 8.00 (s, 1 H), 7.44 (s, 1H), 7.36 (d, 1H), 4.73 (s,1 H).

C. 7-Chloro-2-(4-chloromethyl-thiazole-2-yl)-thieno[3,2-b] pyridine was taken into 2.00 mL ethanol and 400 uL dichloroethane with pyrrolidine and warmed until dissolution was complete. The reaction mixture was allowed to stir overnight and then loaded directly onto silica gel through evaporation. The product, 7-chloro-2-(4-pyrrolidine-1-ylmethyl-thiazol-2-yl)-thieno[3,2-b] pyridine, was isolated via column chromatography (98:2:0.20 CHCl₃:CH₃OH:NH₃OH). C₁₅H₁₄ClN₃S₂: APCI m/z: 336.0/337.1 (MH+); ¹H NMR (CDCl₃): 8.57, (d,1 H), 7.89 (s, 1H), 7.45, (s, 1H), 7.28, (d,1H), 3.96, (s, 2H), 2.80, (s, 4H), 1.88, (s, 4H).

D. (2-methyl-1H-indol-5-yl)-(2-4 pyrrolidin-1-ylmethyl-thiazole-2-yl)-thieno[3,2-b]pyridin-7-yl)-amine was made in a manner similar to Example 25 from 2-methyl-1H-indol-5-yl amine and 7-chloro-2-(4-pyrrolidine-1-ylmethyl-thiazol-2-yl)-thieno[3,2-b] pyridine. C₂₄H₂₃N₅S₂: APCI m/z: 446.2 (MH+), ): ¹H NMR (CD₃OD): 8.11, (d,1 H), 7.88 (s, 1H), 7.45 (s, 1H), 7.33, (d, 1H), 7.28, (d,1H), 6.96, (d,1H), 6.95, (d,1H), 6.62, (d,1H), 6.12, (s, 1H), 3.80, (s,1H), 2.42, (s,3H), 2.65 (vbm, 4H), 1.80 (vbm, 4H).

### EXAMPLE 32

### (2-Methyl-1-H-indol-5-yl)-(2-(4-morpholin-4-ylmethyl-thiazol-2-yl)-thieno[3,2-b]pyridin-7-yl)-amine

A. 7-Chloro-2-(4-morpholin4-ylmethyl-thiazole-2yl)-thieno[3,2-b]pyridine was made from morpholine and 7-chloro-2-(4-chloromethyl-thiazole-2-yl)-thieno[3,2-b] pyridine in a manner similar to Example 31. C₁₅H₁₄ClN₃OS₂: APCI m/z: 352.0/354.0 (MH+), ¹H NMR (CDCl₃): ): 8.57, (bd,1H), 7.90 (s, 1H), 7.29 (s, 1H), 7.28, (d, 1H), 4.72, (s,1H), 3.89, (vbs,4H), 2.88, (vbs, 4H).

B. The title compound was prepared in a manner similar to Example 25 from 2-methyl-1H-indol-5-yl amine and 7-chloro-2-(4-morpholin-4-ylmethyl-thiazole-2yl)-thieno[3,2-b]pyridine. C₂₄H₂₃N₅OS₂: APCI m/z: 461.0/463.0 (MH+), ¹H NMR (CDCl₃): ): 8.57, (bd,1H), 7.90 (s, 1H), 7.29 (s, 1H), 7.28, (d, 1H), 4.72, (bs,4H), 3.89, (bs,4H), 2.88, (vbs, 2H), 2.34, (vbs, 3H).

### EXAMPLE 33

### (2-(7-(2-Methyl-1H-indol-5-ylamino)-thieno[3,2-b]pyridin-2-yl)-thiazol-4-yl)-methanol

A. The title compound was made in a manner analogous to Example 25 from 2-methyl-1H-indol-5-yl amine and 2-(7-chloro-thieno[3,2-b] pyridine-2-yl)-thiazol-4-yl-methanol. C₂₀H₁₆N₄OS₂: APCI m/z: 393.1/395.2 (MH+); HPLC Rf: 4.09 min.

### EXAMPLE 34

### 1-Methyl-5-[7-(2-methyl-1H-indol-5-ylamino)-thieno[3,2-b]pyridin-2-yl]-1H-imidazole-2-carboxylic Acid cyclohexyl-methyl-amide

A. 5-(7-Chloro-thieno[3,2-b]pyridin-2-yl)-1-methyl-1H-imidazole-2-carboxylic acid cyclohexyl-methyl-amide was prepared from 5-(7-chloro-thieno[3,2-b]pyridin-2-yl)-1-methyl-1H-imidazole-2-carboxylic acid methyl ester and cyclohexylmethylamine by a method analogous to Example 3. MS: 389, 391(MH+); HPLC Rf: 6.19 min; HPLC purity 95%

B. The title compound was prepared from 5-(7-chloro-thieno[3,2-b]pyridin-2-yl)-1-methyl-1H-imidazole-2-carboxylic acid cyclohexyl-methyl-amide and 2-methyl-1H-indol-5-ylamine by a procedure analogous to Example 3. MS: 499 (MH+); HPLC Rf: 5.70 min; HPLC purity: 95%.

### EXAMPLE 35

### 1-Methyl-5-[7-(2-methyl-1H-indol-5-ylamino)-thieno[3,2-b]pyridin-2-yl]-1H-imidazole-2-carboxylic Acid (2-dimethylamino-ethyl)-methyl-amide

A. 5-(7-Chloro-thieno[3,2-b]pyridin-2-yl)-1-methyl-1H-imidazole-2-carboxylic acid (2-dimethylamino-ethyl)-methyl-amide was prepared from 5-(7-chloro-thieno[3,2-b]pyridin-2-yl)-1-methyl-1H-imidazole-2-carboxylic acid methyl ester and N-(2-dimethylaminoethyl)methylamine by a method analogous to Example 3. MS: 378, 380 (MH+); HPLC Rf: 4.20 min; HPLC purity 98%

B. The title compound was prepared from 5-(7-chloro-thieno[3,2-b]pyridin-2-yl)-1-methyl-1H-imidazole-2-carboxylic acid (2-dimethylamino-ethyl)-methyl-amide and 2-methyl-1H-indol-5-ylamine by a procedure analogous to Example 3. MS: 488 (MH+); HPLC Rf: 4.09 min; HPLC purity: 99%.

### EXAMPLE 36

### (3,4-Dihydro-1H-isoquinolin-2-yl)-{1-methyl-5-[7-(2-methyl-1H-indol-5-ylamino)-thieno[3,2-b]pyridin-2-yl]-1H-imidazol-2-yl}- methanone

A. [5-(7-Chloro-thieno[3,2-b]pyridin-2-yl)-1-methyl-1H-imidazol-2-yl]-(3,4-dihydro-1H-isoquinolin-2-yl)-methanone was prepared from 5-(7-chloro-thieno[3,2-b]pyridin-2-yl)-1-methyl-1H-imidazole-2-carboxylic acid methyl ester and 3,4-dihydro-1 H-isoquinoline by a method analogous to Example 3. MS: 409, 411 (MH+); HPLC Rf: 6.29 min; HPLC purity 95%

B. The title compound was prepared from [5-(7-chloro-thieno[3,2-b]pyridin-2-yl)-1-methyl-1H-imidazol-2-yl]-(3,4-dihydro-1H-isoquinolin-2-yl)-methanone and 2-methyl-1H-indol-5-ylamine by a procedure analogous to Example 3. MS: 519 (MH+); HPLC Rf: 5.78 min; HPLC purity: 99%.

### EXAMPLE 37

### 1-Methyl-5-[7-(2-methyl-1H-indol-5-ylamino)-thieno[3,2-b]pyridin-2-yl]-1H-imidazole-2-carboxylic acid (2-dimethylamino-ethyl)-amide

A. [5-(7-Chloro-thieno[3,2-b]pyridin-2-yl)-1-methyl-1H-imidazole-2-carboxylic acid (2-dimethylamino-ethyl)-amide was prepared from 5-(7-chloro-thieno[3,2-b]pyridin-2-yl)-1-methyl-1H-imidazole-2-carboxylic acid methyl ester and N-2-dimethylaminoethylamine by a method analogous to Example 3. MS: 364, 366 (MH+); HPLC Rf: 4.22 min; HPLC purity 97%

B. The title compound was prepared from 5-(7-chloro-thieno[3,2-b]pyridin-2-yl)-1-methyl-1H-imidazole-2-carboxylic acid (2-dimethylamino-ethyl)-amide and 2-methyl-1H-indol-5-ylamine by a procedure analogous to Example 3. MS: 474 (MH+); HPLC Rf: 3.69 min; HPLC purity: 99%.

### EXAMPLE 38

### {1-Methyl-5-[7-(2-methyl-1H-indol-5-ylamino)-thieno[3,2-b]pyridin-2-yl]-1H-imidazol-2-yl}-pyrrolidin-1-yl-methanone

A. [5-(7-Chloro-thieno[3,2-b]pyridin-2-yl)-1-methyl-1H-imidazol-2-yl]-pyrrolidin-1-yl-methanone was prepared from 5-(7-chloro-thieno[3,2-b]pyridin-2-yl)-1-methyl-1H-imidazole-2-carboxylic acid methyl ester and pyrrolidine by a method analogous to Example 3. MS: 347, 349 (MH+); HPLC Rf: 5.08 min; HPLC purity 99%

B. The title compound was prepared from [5-(7-chloro-thieno[3,2-b]pyridin-2-yl)-1-methyl-1H-imidazol-2-yl]-pyrrolidin-1-yl-methanone and 2-methyl-1H-indol-5-ylamine by a procedure analogous to Example 3. MS: 457 (MH+); HPLC Rf: 4.53 min; HPLC purity: 95%.

### EXAMPLE 39

### {1-Methyl-5-[7-(2-methyl-1H-indol-5-ylamino)-thieno[3,2-b]pyridin-2-yl]1H-imidazol-2-yl}-2-carboxylic Acid (2,2,2-trifluoroethyl)-amide

A. [5-[7-(Chloro)-thieno[3,2-b]pyridin-2-yl]-1-methyl-1H-imidazol-2-yl}-2-carboxylic acid (2,2,2-trifluoroethyl)-amide was prepared from 5-(7-chloro-thieno[3,2-b]pyridin-2-yl)-1-methyl-1H-imidazole-2-carboxylic acid methyl ester and 2,2,2-trifluoroethylamine by a method analogous to Example 3. MS: 375, 377 (MH+); HPLC Rf: 5.98 min; HPLC purity 92%

B. The title compound was prepared from [5-(7-chloro-thieno[3,2-b]pyridin-2-yl)-1-methyl-1H-imidazol-2-yl]-2-carboxylic acid (2,2,2-trifluoroethyl)-amide and 2-methyl-1H-indol-5-ylamine by a procedure analogous to Example 3. MS: 485 (MH+); HPLC Rf: 4.61 min; HPLC purity: 94%.

### EXAMPLE 40

### 3-Methyl-2-[7-(2-methyl-1H-indol-5-ylamino)-thieno[3,2-b]pyridin-2-yl]-3H-imidazole-4-carboxylic Acid Dimethylamide

A. A solution of 7-chloro-2-(1-methyl-1H-imidazol-2-yl)-thieno[3,2-b]pyridine (250 mg, 1.0 mmol) in anhydrous THF (30 mL) was cooled to −78 C via dry ice/acetone bath. n-BuLi solution (2.5 M in hexanes, 440 uL, 1.1 mmol) was added slowly. The solution was stirred at −78 C. for 30 minutes. Methyl chloroformate (190 mg, 2.0 mmole) was added dropwise, resulting in a precipitation of white solids. The reaction mixture was stirred at −78 C. for 30 minutes and the dry ice/acetone bath was removed. After warming to room temperature, the reaction mixture was diluted with methanol. The crude material was concentrated on 1 gram of silica gel by removing the solvent in vacuo. The residue was purified eluting dichloromethane:methanol (100:3) through a Biotage FLASH40M cartridge. 2-(7-Chloro-thieno[3,2-b]pyridin-2-yl)-3-methyl-3H-imidazole-4-carboxylic acid methyl ester was obtained as a white solid (108 mg, 0.35 mmol, 35%).

B. 2-(7-Chloro-thieno[3,2-b]pyridin-2-yl)-3-methyl-3H-imidazole-4-carboxylic acid dimethylamide was prepared from dimethylamine and 2-(7-chloro-thieno[3,2-b]pyridin-2-yl)-3-methyl-3H-imidazole-4-carboxylic acid methyl ester by a procedure analogous to Example 3. MS: 321, 232 (MH+); HPLC Rf: 4.27 min.; HPLC purity 91%.

C. The title compound was prepared from 2-(7-chloro-thieno[3,2-b]pyridin-2-yl)-3-methyl-3H-imidazole-4-carboxylic acid dimethylamide and 2-methyl-1H-indol-5-ylamine by a procedure analogous to Example 3. MS: 431 (MH+), HPLC Rf: 4.01 min.; HPLC purity: 99%.

### EXAMPLE 41

### 2-{3-Methyl-2-[7-(2-methyl-1H-indol-5-ylamino)-thieno[3,2-b]pyridin-2-yl]-3H-imidazol-4-yl}-propane-1,2-diol

A. A solution of 7-chloro-2-(1-methyl-1H-imidazol-2-yl)-thieno[3,2-b]pyridine (250 mg, 1.0 mmol) in anhydrous THF (30 mL) was cooled to −78 C. via dry ice/acetone bath. n-BuLi solution (2.5 M in hexanes, 440 uL, 1.1 mmol) was added slowly, and the solution was stirred at −78 C. for 30 minutes. 1-(tert-Butyldimethylsilyloxy)-2-propane (380 mg, 2.0 mmol) was added dropwise. The reaction mixture was stirred at −78 C. for 30 minutes and the dry ice/acetone bath was removed. After warming to room temperature, the reaction mixture was diluted with methanol. The crude material was concentrated onto 1 gram of silica gel by removing the solvent in vacuo. The dry silica gel powder was eluted with dichloromethane:methanol (100:3) through a Biotage FLASH40M cartridge. 1-(tert-Butyl-dimethyl-silanyloxy)-2-[2-(7-chloro-thieno[3,2-b]pyridin-2-yl)-3-methyl-3H-imidazol-4-yl]-propan-2-ol was obtained as a white solid (176 mg, 0.40 mmol, 40%). %). MS: 438, 440 (MH+); HPLC Rf: 7.99 min.; HPLC purity 99%.

B. The title compound was prepared from 1-(tert-butyl-dimethyl-silanyloxy)-2-[2-(7-chloro-thieno[3,2-b]pyridin-2-yl)-3-methyl-3H-imidazol-4-yl]-propan-2-ol and 2-methyl-1H-indol-5-ylamine by a procedure analogous to Example 3 (this reaction introduces the indole and cleaves the tert-butyldimethylsilyl group simultaneously). MS:434 (MH+); HPLC Rf: 3.73 min.; HPLC purity 95%.

### EXAMPLE 42

### (2-Methyl-1H-indol-5-yl)-(2-oxazol-5-yl-thieno[3,2-b]pyridin-7-yl)-amine

A. 7-Chloro-thieno[3,2-b]pyridine-2-carbaldehyde.

To a solution of 7-chloro-thieno[3,2-b]pyridine (20g, 0.118 mol) in THF (240 ml) was added n-butyl lithium in hexane (2.5 M, 59 ml, 147 mmol) at −78° C. under an atmosphere of dry N₂. The mixture was stirred at −78° C. for 90 minutes. N,N-dimethylformamide (DMF, 27 ml, 354 mmol) was added at the same temperature. The solution was stirred at −78° C. for 2 hours and then quenched with a saturated aqueous solution of ammonium chloride. The reaction mixture was poured into 1000 ml water resulting in white precipitate. After filtration, and washing with ethyl ether, the product was obtained as a white solid (16.83 g, 72% yield).

¹H NMR (CDCl3) 10.25 (s, 1H), 8.76 (d, IH, J=4.98 Hz), 7.48 (d, 1 H, J=4.98 Hz), 7.39 (s, 1 H); HPLC Rf: 7.809 min.

B. 7-Chloro-2-oxazol-5-yl-thieno[3,2-b]pyridine

To a solution of 7-chloro-thieno[3,2-b]pyridine-2-carbaldehyde (0.50 g, 2.54 mmol) in methanol (10 ml) was added tosylmethyl isocynide (TOSMIC, 0.495 g, 2.54 mmol) followed by addition of potassium carbonate (0.737 g, 5.33 mmol) at room temperature under an atmosphere of dry N₂. The mixture was heated at 70° C. for 90 minutes. After cooling to room temperature and filtration, the white precipitate was collected to afford the title compound. C.I. MS: m/z 237.0 [M+1].

C. (2-Methyl-1H-indol-5-yl)-(2-oxazol-5-yl-thieno[3,2-b]pyridin-7-yl)-amine

To a solution of 7-chloro-2-oxazol-5-yl-thieno[3,2-b]pyridine (0.49 g, 2.09 mmol) in ethanol (6 ml) was added 2-methyl-1H-indol-5-ylamine (0.456 g, 3.11 mmol). The reaction was heated at 85° C. for 60 hours. Evaporated to remove the solvent. The residue was chromatographed on silica gel with MeOH—CHCl₃—NH₄OH (2:99:0.1 to 7:93:0.1) as eluents to afford the title compound (197 mg, 0.569 mmol, 27% yield). CI-MS: m/z 347.1 [M+1].

### EXAMPLE 43

### (2-Methyl-1H-indol-5-yl)-[2-(5-methyl-oxazol-2-yl)-thieno[3,2-b]pyridin-7-yl]-amine

A. 7-Chloro-thieno[3,2-b]pyridine-2-carboxylic acid (2-oxo-propyl)-amide

To a solution of 1-amino-propan-2-one hydrochloride (0.47 g, 4.33 mmol) in dichloromethane (8 ml) was added triethyl amine (1.26 ml, 9.09 mmol). After strring for 10 minutes, a solution of 7-chloro-thieno[3,2-b]pyridine-2-carbonyl chloride (1.0 g, 4.33 mmol) in dichloromethane (4 ml) was added dropwise at 0° C. under an atmosphere of dry N₂. The mixture was stirred at ambient temperature for 90 minutes and then was quenched with water followed by addition of chloroform. After separation, the organic layer was washed with brine, dried over MgSO₄ and concentrated under vacuum to yield the crude product. It was chromatographed on silica gel with MeOH—CHCl₃—NH₄OH (2:98:0.1 to 5:95:0.1) as eluents to afford the title compound (355 mg, 30.6% yield). CI-MS: mlz 269.1 HPLC Rf: 5.908 min.

B. 7-Chloro-2-(5-methyl-oxazol-2-yl)-thieno[3,2-b]pyridine

7-Chloro-thieno[3,2-b]pyridine-2-carboxylic acid (2-oxo-propyl)-amide (50 mg, 0.187 mmol) was dissolved in 0.5 ml of concentrated sulfuric acid. The mixture was heated at 80° C. for 4 hours. After cooling to room temperature, the mixture was poured onto ice water. The solution was adjust to pH 9 and was extracted with ethyl acetate. After separation, the organic layer was washed with brine, dried over MgSO₄ and concentrated under vacuum to yield the title compound (39 mg, 83.6 % yield). CI-MS: m/z 251.0 HPLC Rf: 9.20 min.

C. 2-Methyl-1H-indol-5-yl)-[2-(5-methyl-oxazol-2-yl)-thieno[3,2-b]pyridin-7-yl]-amine

To a solution of 7-chloro-2-(5-methyl-oxazol-2-yl)-thieno[3,2-b]pyridine (0.063 g, 0.252 mmol) in ethanol (1 ml) was added 2-methyl-1H-indol-5-ylamine (0.074 g, 0.504 mmol). The reaction mixture was heated at 85° C. for 15 hours. Evaporated to remove the solvent. The residue was chromatographed on silica gel with MeOH—CHCl₃—NH₄OH (2:99:0.1 to 7:93:0.1) as eluents to afford the title compound (30 mg, 33% yield). CI-MS: m/z 361.1 [M +1]. HPLC Rf: 6.728 min.

1. A compound of the formula 1 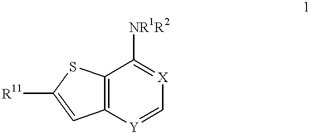
or a pharmaceutically acceptable salt, prodrug or hydrate thereof,
X is N, CH or C—CN;
Y is N, CH, CF, or N→O;
R¹ is H;
R² is 5 to 13 membered heterocyclic, wherein said R² group is optionally substituted by 1 to 5 R⁵ substituents,
each R⁵ is independently selected from halo, cyano, trifluoromethoxy, trifluoromethyl, —C(O)R⁸, —NR⁶C(O)R⁷, —C(O)NR⁶R⁷, —NR⁶R⁷, —(CH₂)ₜNR⁶R⁷, —OR⁹, —SO₂NR⁶R⁷, —NR⁹SO₂NR⁶R⁷, —SO₂R⁶, C₁-C₆ alkyl, C₂-C₆ alkenyl, —(CH₂)ⱼO(CH₂)_{q}NR⁶R⁷, —(CH₂)ₜO(CH₂)_{q}OR⁹, —(CH₂)ₜOR⁹, —S(O)ⱼ(C₁-C₆ alkyl), —(CH₂)ₜ(C₆-C₁₀ aryl), —(CH₂)ₜ(5 to 10 membered heterocyclic), —(CH₂)ₜO(CH₂)_{q}(5 to 10 membered heterocyclic), —C(O)(CH₂)ₜ(5 to 10 membered heterocyclic), —(CH₂)ⱼNR⁷(CH₂)_{q}NR⁶R⁷, —(CH₂)ⱼNR⁷CH₂C(O)NR⁶R⁷, —(CH₂)ⱼNR⁷(CH₂)_{q}NR⁹C(O)R⁸, —(CH₂)ⱼNR⁷(CH₂)ₜO(CH₂)_{q}OR⁹, —(CH₂)ⱼNR⁷(CH₂)_{q}S(O)ⱼ(C₁-C₆ alkyl), —(CH₂)ⱼNR⁷(CH₂)ₜR⁶, —SO₂(CH₂)ₜ(C₆-C₁₀ aryl), and —SO₂(CH₂)ₜ(5 to 10 membered heterocyclic), wherein j is an integer from 0 to 2, t is an integer from 0 to 6, q is an integer from 2 to 6, the —(CH₂)_{q}— and —(CH₂)ₜ— moieties of the foregoing R⁵ groups optionally include a carbon-carbon double or triple bond where t is an integer from 2 to 6, and the alkyl, aryl and heterocyclic moieties of the foregoing R⁵ groups are optionally substituted by 1 to 3 substituents independently selected from halo, cyano, trifluoromethyl, —C(O)R⁸, —(CH₂)ₜOR⁹, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6;
each R⁶ and R⁷ is independently selected from H, C₁-C₆ alkyl, —(CH₂)ₜ(C₆-C₁₀ aryl), —(CH₂)ₜ(C₆-C₁₀ cycloalkyl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, and the alkyl, aryl and heterocyclic moieties of the foregoing R⁶ and R⁷ groups are optionally substituted by 1 to 3 substituents independently selected from halo, cyano, trifluoromethyl, —C(O)R⁸, —NR⁹C(O)R¹⁰, —C(O)NR⁹R¹⁰, —NR⁹R¹⁰, C₁-C₆ alkyl, —(CH₂)ₜ(C₆-C₁₀ aryl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, with the proviso that where R⁶ and R⁷ are both attached to the same nitrogen, then R⁶ and R⁷ are not both bonded to the nitrogen directly through an oxygen;
each R⁸ is independently selected from H, C₁-C₁₀ alkyl, —O(C₁-C₁₀ alkyl), —(CH₂)ₜ(C₆-C₁₀ aryl), and —(CH₂)ₜ(5 to 10 membered heterocyclic), wherein t is an integer from 0 to 6;
each R⁹ and R¹⁰ is independently selected from H and C₁-C₆ alkyl; and,
R¹¹ is selected from the group consisting of imidazolyl, oxazolyl, oxadiazolyl, isoxazolyl, thiazolyl and thiadiazolyl, wherein said imidazolyl, oxazolyl, oxadiazolyl, isoxazolyl, thiazolyl and thiadiazolyl are optionally substituted by 1 to 5 R⁶ groups with the proviso that compound 1 is not
[2-(3-Methyl-3H-imidazol-4-yl)-thieno[3,2-b]pyridin-7-yl]-(2-methyl-1H-indol-5-yl)-amine;
2-{1-Methyl-5-[7-(2-methyl-1H-indol-5-ylamino)-thieno[3,2-b]pyridin-2-yl]-1H-imidazol-2-yl}-propan-2-ol;
[2-(1-Methyl-1H-imidazol-2-yl)-thieno[3,2-b]pyridin-7-yl]-(2-methyl-1H-indol-5-yl)-amine;
(2-Methyl-1H-indol-5-yl)-(2-thiazol-2-yl-thieno[3,2-b]pyridin-7-yl)-amine; or
2-{2-[7-(2-Methyl-1H-indol-5-ylamino)-thieno[3,2-b]pyridin-2-yl]-thiazol-5-yl}-propan-2-ol.

2. The compound of claim 1, wherein R¹¹ is imidazolyl, oxazolyl or thiazolyl, wherein said imidazolyl, oxazolyl and thiazolyl are optionally substituted by 1 to 5 R⁵ groups.

3. The compound of claim 2, wherein said imidazolyl, oxazolyl and thiazolyl are optionally substituted by 1 to 5 R⁵ groups, each R⁵ is independently selected from cyano, —C(O)R⁸, —NR⁶C(O)R⁷, —C(O)NR⁶R⁷, —NR⁶R⁷, —OR⁹, C₁-C₆ alkyl, —(CH₂)ⱼO(CH₂)_{q}NR⁶R⁷, —(CH₂)ₜO(CH₂)_{q}OR⁹, —(CH₂)ₜOR⁹, —(CH₂)ₜ(5 to 10 membered heterocyclic), —C(O)(CH₂)ₜ(5 to 10 membered heterocyclic), —(CH₂)ⱼNR⁷(CH₂)_{q}NR⁶R⁷, —(CH₂)ⱼNR⁷CH₂C(O)NR⁶R⁷ —(CH₂)ⱼNR⁷(CH₂)_{q}NR⁹C(O)R⁸, —(CH₂)ⱼNR⁷(CH₂)ₜO(CH₂)_{q}OR⁹, and —(CH₂)ⱼNR⁷(CH₂)ₜR⁶, wherein j is an integer from 0 to 2, t is an integer from 0 to 6, q is an integer from 2 to 6, the —(CH₂)_{q}— and —(CH₂)ₜ— moieties of the foregoing R⁵ groups optionally include a carbon-carbon double or triple bond where t is an integer from 2 to 6, and the alkyl, aryl and heterocyclic moieties of the foregoing R⁵ groups are optionally substituted by 1 to 3 substituents independently selected from halo, cyano, trifluoromethyl, —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

4. The compound of claim 3, wherein each R⁵ is independently selected from —C(O)R⁸, —C(O)NR⁶R⁷, —NR⁶R⁷, —OR⁹, C₁-C₆ alkyl, —C(O)(CH₂)ₜ(5 to 10 membered heterocyclic), wherein t is an integer from 0 to 6, the —(CH₂)ₜ— moiety of the foregoing R⁵ group optionally includes a carbon-carbon double or triple bond when t is an integer from 2 to 6, and the alkyl and heterocyclic moieties of the foregoing R⁵ groups are optionally substituted by 1 to 3 substituents independently selected from halo, cyano, trifluoromethyl, —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

5. The compound of claim 4, wherein each R⁵ is independently selected from —C(O)R⁸, —C(O)NR⁶R⁷, C₁-C₆ alkyl, —C(O)(CH₂)ₜ(5 to 10 membered heterocyclic), wherein t is an integer from 0 to 6, the —(CH₂)ₜ— moiety of the foregoing R⁵ group optionally includes a carbon-carbon double or triple bond when t is an integer from 2 to 6, and the alkyl and heterocyclic moieties of the foregoing R⁵ groups are optionally substituted by 1 to 3 substituents independently selected from —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

6. The compound of claim 1, wherein R² is a group of the formula 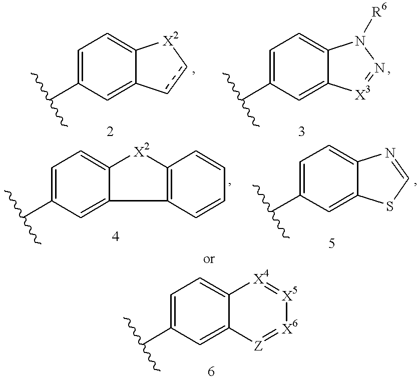
wherein X² is —S—, —N(R⁶)— or O, and X³, X⁴, X⁵, X⁶, and Z is N or CH, the dashed line in formula 2 represents an optional double bond, and the above R² groups of formulas 2, 4 and 6 are optionally substituted by 1 to 5 R⁵ substituents and the R² groups of formulas 3 and 5 are optionally substituted by 1 to 3 R⁵ substituents.

7. The compound of claim 6, wherein said R² group is a group of formula 2, wherein said group is optionally substituted by 1 to 3 R⁵ substituents.

8. The compound of claim 1, wherein said compound is selected from the group consisting of:
{1-Methyl-5-[7-(2-methyl-1H-indol-5-ylamino)-thieno[3,2-b]pyridin-2-yl]-1H-imidazol-2-yl}-morpholin-4-yl-methanone;
{1-Methyl-5-[7-(2-methyl-1H-indol-5-ylamino)-thieno[3,2-b]pyridin-2-yl]-1H-imidazol-2-yl}-(4-methyl-piperazin-1-yl)-methanone;
1-Methyl-5-[7-(2-methyl-1H-indol-5-ylamino)-thieno[3,2-b]pyridin-2-yl]-1H-imidazole-2-carboxylic acid dimethylamide;
1-Methyl-5-[7-(2-methyl-1H-indol-5-ylamino)-thieno[3,2-b]pyridin-2-yl]-1H-imidazole-2-carboxylic acid methylamide;
2-{1-Methyl-5-[7-(2-methyl-1H-indol-5-ylamino)-thieno[3,2-b]pyridin-2-yl]-1H-imidazol-2-yl}-propane-1,2-diol;
1-Methyl-5-[7-(2-methyl-1H-indol-5-ylamino)-thieno[3,2-b]pyridin-2-yl]-1H-imidazole-2-carboxylic acid amide;
2-{2-[7-(2-Methyl-1H-indol-5-ylamino)-thieno[3,2-b]pyridin-2-yl]-thiazol-4-yl}-propan-2-ol;
(2-methyl-1H-indol-5-yl)-(2-4 pyrrolidin-1-ylmethyl-thiazole-2-yl)-thieno[3,2-b]pyridin-7-yl)-amine; pharmaceutically acceptable salts of said compounds; solvates of said compounds; and prodrugs of said compounds.

9. A compound of the formula 1 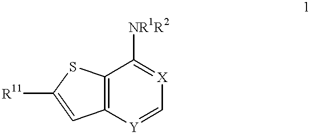
or a pharmaceutically acceptable salt, prodrug or hydrate thereof,
X is N, CH or C—CN;
Y is N, CH, CF, or N→O;
R¹ is H;
R² is 5 to 13 membered heterocyclic, wherein said R² group is optionally substituted by 1 to 5 R⁵ substituents,
each R⁵ is independently selected from halo, cyano, trifluoromethoxy, trifluoromethyl, —C(O)R⁸, —NR⁶C(O)R⁷, —C(O)NR⁶R⁷, —NR⁶R⁷, —(CH₂)ₜNR⁶R⁷, —OR⁹, —SO₂NR⁶R⁷, —NR⁹SO₂NR⁶R⁷, —SO₂R⁶, C₁-C₆ alkyl, C₂-C₆ alkenyl, —(CH₂)ⱼO(CH₂)_{q}NR⁶R⁷, —(CH₂)ₜO(CH₂)_{q}OR⁹, —(CH₂)ₜOR⁹, —S(O)ⱼ(C₁-C₆ alkyl), —(CH₂)ₜ(C₆-C₁₀ aryl), —(CH₂)ₜ(5 to 10 membered heterocyclic), —(CH₂)ₜO(CH₂)_{q}(5 to 10 membered heterocyclic), —C(O)(CH₂)ₜ(5 to 10 membered heterocyclic), —(CH₂)ⱼNR⁷(CH₂)_{q}NR⁶R⁷, —(CH₂)ⱼNR⁷CH₂C(O)NR⁶R⁷, —(CH₂)ⱼNR⁷(CH₂)_{q}NR⁹C(O)R⁸, —(CH₂)ⱼNR⁷(CH₂)ₜO(CH₂)_{q}OR⁹, —(CH₂)ⱼNR⁷(CH₂)_{q}S(O)ⱼ(C₁-C₆ alkyl), —(CH₂)ⱼNR⁷(CH₂)ₜR⁶, —SO₂(CH₂)ₜ(C₆-C₁₀ aryl), and —SO₂(CH₂)ₜ(5 to 10 membered heterocyclic), wherein j is an integer from 0 to 2, t is an integer from 0 to 6, q is an integer from 2 to 6, the —(CH₂)_{q}— and —(CH₂)ₜ— moieties of the foregoing R⁵ groups optionally include a carbon-carbon double or triple bond where t is an integer from 2 to 6, and the alkyl, aryl and heterocyclic moieties of the foregoing R⁵ groups are optionally substituted by 1 to 3 substituents independently selected from halo, cyano, trifluoromethyl, —C(O)R⁸, —(CH₂)ₜOR⁹, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), (CH₂)ₜO(CH₂)_{q}OR⁹, and —(CH₂)ₜOR⁹, wherein t is an integer from 0 to 6 and q is an integer from 2 to 6;
each R⁶ and R⁷ is independently selected from H, C₁-C₆ alkyl, —(CH₂)ₜ(C₆-C₁₀ aryl), —(CH₂)ₜ(C₆-C₁₀ cycloalkyl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, and the alkyl, aryl and heterocyclic moieties of the foregoing R⁶ and R⁷ groups are optionally substituted by 1 to 3 substituents independently selected from halo, cyano, trifluoromethyl, —C(O)R⁸, —NR⁹C(O)R¹⁰, —C(O)NR⁹R¹⁰, —NR⁹R¹⁰, C₁-C₆ alkyl, —(CH₂)ₜ(C₆-C₁₀ aryl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, or with the proviso that where R⁶ and R⁷ are both attached to the same nitrogen, then R⁶ and R⁷ are not both bonded to the nitrogen directly through an oxygen;
each R⁸ is independently selected from H, C₁-C₁₀ alkyl, —O(C₁-C₁₀ alkyl), —(CH₂)ₜ(C₆-C₁₀ aryl), and —(CH₂)ₜ(5 to 10 membered heterocyclic), wherein t is an integer from 0 to 6;
each R⁹ and R¹⁰ is independently selected from H and C₁-C₆ alkyl; and,
R¹¹ is selected from the group consisting of imidazolyl, oxazolyl, oxadiazolyl, isoxazolyl, thiazolyl and thiadiazolyl, wherein said imidazolyl, oxazolyl, oxadiazolyl, isoxazolyl, thiazolyl and thiadiazolyl are optionally substituted by 1 to 5 R⁵ groups with the proviso that compound 1 is not
[2-(3-Methyl-3H-imidazol-4-yl)-thieno[3,2-b]pyridin-7-yl]-(2-methyl-1H-indol-5-yl)-amine;
2-{1-Methyl-5-[7-(2-methyl-1H-indol-5-ylamino)-thieno[3,2-b]pyridin-2-yl]-1H-imidazol-2-yl}-propan-2-ol;
[2-(1-Methyl-1H-imidazol-2-yl)-thieno[3,2-b]pyridin-7-yl]-(2-methyl-1H-indol-5-yl)-amine;
(2-Methyl-1H-indol-5-yl)-(2-thiazol-2-yl-thieno[3,2-b]pyridin-7-yl)-amine;
2-{2-[7-(2-Methyl-1H-indol-5-ylamino)-thieno[3,2-b]pyridin-2-yl]-thiazol-5-yl}-propan-2-ol;
[2-(2-Ethoxy-thiazol-5-yl)-thieno[3,2-b]pyridin-7-yl]-(2-methyl-1H-indol-5-yl)-amine;
(2-Methyl-1H-indol-5-yl)-[2-(4-methyl-thiazol-2-yl)-thieno[3,2-b]pyridin-7-yl]-amine;
[2-(3-Methoxymethyl-3H-imidazol-4-yl)-thieno[3,2-b]pyridin-7-yl]-(2-methyl-1H-indol-5-yl)-amine;
{2-[5-(4-Methoxy-phenyl)-oxazol-2-yl}-thieno[3,2-b]pyridin-7-yl]-(2-methyl-1H-indol-5-yl)-amine; or
2-{4-Methyl-2-[7-(2-methyl-1H-indol-5-ylamino)-thieno[3,2-b]pyridin-2-yl]-thiazol-5-yl}-propan-2-ol.

10. The compound of claim 9, wherein R¹¹ is imidazolyl, oxazolyl or thiazolyl, wherein said imidazolyl, oxazolyl and thiazolyl are optionally substituted by 1 to 5 R⁵ groups.

11. The compound of claim 10, wherein said imidazolyl, oxazolyl and thiazolyl are optionally substituted by 1 to 5 R⁵ groups, each R⁵ is independently selected from cyano, —C(O)R⁸, —NR⁶C(O)R⁷, —C(O)NR⁶R⁷, —NR⁶R⁷, —OR⁹, C₁-C₆ alkyl, —(CH₂)ⱼO(CH₂)_{q}NR⁶R⁷, —(CH₂)ₜO(CH₂)_{q}OR⁹, —(CH₂)ₜOR⁹, —(CH₂)ₜ(5 to 10 membered heterocyclic), —C(O)(CH₂)ₜ(5 to 10 membered heterocyclic), —(CH₂)ⱼNR⁷(CH₂)_{q}NR⁶R⁷, —(CH₂)ⱼNR⁷CH₂C(O)NR⁶R⁷, —(CH₂)ⱼNR⁷(CH₂)_{q}NR⁹C(O)R⁸, —(CH₂)ⱼNR⁷(CH₂)ₜO(CH₂)_{q}OR⁹, and —(CH₂)ⱼNR⁷(CH₂)ₜR⁶, wherein j is an integer from 0 to 2, t is an integer from 0 to 6, q is an integer from 2 to 6, the —(CH₂)_{q}— and —(CH₂)ₜ— moieties of the foregoing R⁵ groups optionally include a carbon-carbon double or triple bond where t is an integer from 2 to 6, and the alkyl, aryl and heterocyclic moieties of the foregoing R⁵ groups are optionally substituted by 1 to 3 substituents independently selected from halo, cyano, trifluoromethyl, —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

12. The compound of claim 11, wherein each R⁵ is independently selected from —C(O)R⁸, —C(O)NR⁶R⁷, —NR⁶R⁷, —OR⁹, C₁-C₆ alkyl, —C(O)(CH₂)ₜ(5 to 10 membered heterocyclic), wherein t is an integer from 0 to 6, the —(CH₂)ₜ— moiety of the foregoing R⁵ group optionally includes a carbon-carbon double or triple bond when t is an integer from 2 to 6, and the alkyl and heterocyclic moieties of the foregoing R⁵ groups are optionally substituted by 1 to 3 substituents independently selected from halo, cyano, trifluoromethyl, —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

13. The compound of claim 12, wherein each R⁵ is independently selected from —C(O)R⁸, —C(O)NR⁶R⁷, C₁-C₆ alkyl, —C(O)(CH₂)ₜ(5 to 10 membered heterocyclic), wherein t is an integer from 0 to 6, the —(CH₂)ₜ— moiety of the foregoing R⁵ group optionally includes a carbon-carbon double or triple bond when t is an integer from 2 to 6, and the alkyl and heterocyclic moieties of the foregoing R⁵ groups are optionally substituted by 1 to 3 substituents independently selected from —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

14. The compound of claim 9, wherein R² is a group of the formula 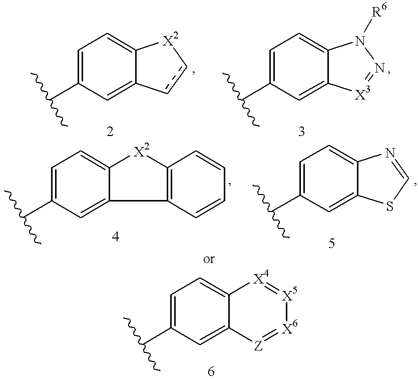
wherein X² is —S—, —N(R⁶)— or O, and X³, X⁴, X⁵, X⁶, and Z is N or CH, the dashed line in formula 2 represents an optional double bond, and the above R² groups of formulas 2, 4 and 6 are optionally substituted by 1 to 5 R⁵ substituents and the R² groups of formulas 3 and 5 are optionally substituted by 1 to 3 R⁵ substituents.

15. The compound of claim 14, wherein said R² group is a group of formula 2, wherein said group is optionally substituted by 1 to 3 R⁵ substituents.

16. The compound of claim 9, wherein said compound is selected from the group consisting of:
{1-Methyl-5-[7-(2-methyl-1H-indol-5-ylamino)-thieno[3,2-b]pyridin-2-yl]-1H-imidazol-2-yl}-morpholin-4-yl-methanone;
{1-Methyl-5-[7-(2-methyl-1H-indol-5-ylamino)-thieno[3,2-b]pyridin-2-yl]-1H-imidazol-2-yl}-(4-methyl-piperazin-1-yl)-methanone;
1-Methyl-5-[7-(2-methyl-1H-indol-5-ylamino)-thieno[3,2-b]pyridin-2-yl]-1H-imidazole-2-carboxylic acid dimethylamide;
1-Methyl-5-[7-(2-methyl-1H-indol-5-ylamino)-thieno[3,2-b]pyridin-2-yl]-1H-imidazole-2-carboxylic acid methylamide;
2-{1-Methyl-5-[7-(2-methyl-1H-indol-5-ylamino)-thieno[3,2-b]pyridin-2-yl]-1H-imidazol-2-yl}-propane-1,2-diol;
1-Methyl-5-[7-(2-methyl-1H-indol-5-ylamino)-thieno[3,2-b]pyridin-2-yl]-1H-imidazole-2-carboxylic acid amide;
2-{2-[7-(2-Methyl-1H-indol-5-ylamino)-thieno[3,2-b]pyridin-2-yl]-thiazol-4-yl}-propan-2-ol;
(2-methyl-1H-indol-5-yl)-(2-4 pyrrolidin-1-ylmethyl-thiazole-2-yl)-thieno[3,2-b]pyridin-7-yl)-amine; pharmaceutically acceptable salts of said compounds; solvates of said compounds; and prodrugs of said compounds.

17. A compound of the formula 1 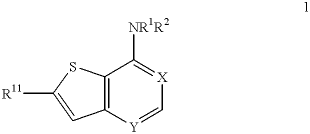
or a pharmaceutically acceptable salt, prodrug or hydrate thereof,
X is CH;
Y is N;
R¹ is H;
R² is 5 to 13 membered heterocyclic, wherein said R²group is optionally substituted by 1 to 5 R⁵ substituents,
each R⁵ is independently selected from cyano, —C(O)R⁸, —NR⁶C(O)R⁷, —C(O)NR⁶R⁷, —NR⁶R⁷, —OR⁹, —NR⁹SO₂NR⁶R⁷, —SO₂R⁶, C₁-C₆ alkyl, —(CH₂)ⱼO(CH₂)_{q}NR⁶R⁷, —(CH₂)ₜO(CH₂)_{q}OR⁹, —(CH₂)ₜOR⁹, —(CH₂)ₜ(5 to 10 membered heterocyclic), —C(O)(CH₂)ₜ(5 to 10 membered heterocyclic), —(CH₂)ⱼNR⁷(CH₂)_{q}NR⁶R⁷, —(CH₂)ⱼNR⁷CH₂C(O)NR⁶R⁷, —(CH₂)ⱼNR⁷(CH₂)_{q}NR⁹C(O)R⁸, —(CH²)ⱼNR⁷(CH₂)ₜO(CH₂)_{q}OR⁹, and —(CH₂)ⱼNR⁷(CH₂)ₜR⁶, wherein j is an integer from 0 to 2, t is an integer from 0 to 6, q is an integer from 2 to 6, the —(CH₂)_{q}— and —(CH₂)ₜ— moieties of the foregoing R⁵ groups optionally include a carbon-carbon double or triple bond where t is an integer from 2 to 6, and the alkyl, aryl and heterocyclic moieties of the foregoing R⁵ groups are optionally substituted by 1 to 3 substituents independently selected from halo, cyano, trifluoromethyl, —C(O)R⁸, —NR⁸C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6;
each R⁶ and R⁷ is independently selected from H, C₁-C₆ alkyl, —(CH₂)ₜ(C₆-C₁₀ aryl), —(CH₂)ₜ(C₆-C₁₀ cycloalkyl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, and the alkyl, aryl and heterocyclic moieties of the foregoing R⁶ and R⁷ groups are optionally substituted by 1 to 3 substituents independently selected from halo, cyano, trifluoromethyl, —C(O)R⁸, —NR⁹C(O)R¹⁰, —C(O)NR⁹R¹⁰, —NR⁹R¹⁰, C₁-C₆ alkyl, —(CH₂)ₜ(C₆-C₁₀ aryl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, with the proviso that where R⁶ and R⁷ are both attached to the same nitrogen, then R⁶ and R⁷ are not both bonded to the nitrogen directly through an oxygen;
each R⁸ is independently selected from H, C₁-C₁₀ alkyl, —O(C₁-C₁₀ alkyl), —(CH₂)ₜ(C₆-C₁₀ aryl), and —(CH₂)ₜ(5 to 10 membered heterocyclic), wherein t is an integer from 0 to 6;
each R⁹ and R¹⁰ is independently selected from H and C₁-C₆ alkyl; and,
R¹¹ is selected from the group consisting of imidazolyl, oxazolyl, oxadiazolyl, isoxazolyl, thiazolyl and thiadiazolyl, wherein said imidazolyl, oxazolyl, oxadiazolyl, isoxazolyl, thiazolyl and thiadiazolyl are optionally substituted by 1 to 5 R⁵ groups with the proviso that compound 1 is not
[2-(3-Methyl-3H-imidazol-4-yl)-thieno[3,2-b]pyridin-7-yl]-(2-methyl-1H-indol-5-yl)-amine;
2-{1-Methyl-5-[7-(2-methyl-1H-indol-5-ylamino)-thieno[3,2-b]pyridin-2-yl]-1H-imidazol-2-yl}-propan-2-ol;
[2-(1-Methyl-1H-imidazol-2-yl)-thieno[3,2-b]pyridin-7-yl]-(2-methyl-1H-indol-5-yl)-amine;
(2-Methyl-1H-indol-5-yl)-(2-thiazol-2-yl-thieno[3,2-b]pyridin-7-yl)-amine; or 2-{2-[7-(2-Methyl-1H-indol-5-ylamino)-thieno[3,2-b]pyridin-2-yl]-thiazol-5-yl}-propan-2-ol.

18. The compound of claim 17, wherein each R⁵ is independently selected from cyano, —C(O)R⁸, —NR⁶C(O)R⁷, —C(O)NR⁶R⁷, —NR⁶R⁷, —OR⁹, C₁-C₆ alkyl, (CH₂)ⱼO(CH₂)_{q}NR⁶R⁷, —(CH₂)ₜO(CH₂)_{q}OR⁹, —(CH₂)ₜOR⁹, —(CH₂)ₜ(5 to 10 membered heterocyclic), —C(O)(CH₂)ₜ(5 to 10 membered heterocyclic), —(CH₂)ⱼNR⁷(CH₂)_{q}NR⁶R⁷, —(CH₂)ⱼNR⁷CH₂C(O)NR⁶R⁷, —(CH₂)ⱼNR⁷(CH₂)_{q}NR⁹C(O)R⁸, —(CH₂)ⱼNR⁷(CH₂)ₜO(CH₂)_{q}OR⁹, and —(CH₂)ⱼNR⁷(CH₂)ₜR⁶, wherein j is an integer from 0 to 2, t is an integer from 0 to 6, q is an integer from 2 to 6, the —(CH₂)_{q}— and —(CH₂)ₜ— moieties of the foregoing R⁵ groups optionally include a carbon-carbon double or triple bond where t is an integer from 2 to 6, and the alkyl, aryl and heterocyclic moieties of the foregoing R⁵ groups are optionally substituted by 1 to 3 substituents independently selected from halo, cyano, trifluoromethyl, —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

19. The compound of claim 18, wherein each R⁵ is independently selected from —C(O)R⁸, —C(O)NR⁶R⁷, —NR⁶R⁷, —OR⁹, C₁-C₆ alkyl, —C(O)(CH₂)ₜ(5 to 10 membered heterocyclic), wherein t is an integer from 0 to 6, the —(CH₂)ₜ— moiety of the foregoing R⁵ group optionally includes a carbon-carbon double or triple bond when t is an integer from 2 to 6, and the alkyl and heterocyclic moieties of the foregoing R⁵ groups are optionally substituted by 1 to 3 substituents independently selected from halo, cyano, trifluoromethyl, —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

20. The compound of claim 19, wherein each R⁵ is independently selected from —C(O)R⁸, —C(O)NR⁶R⁷, C₁-C₆ alkyl, —C(O)(CH₂)ₜ(5 to 10 membered heterocyclic), wherein t is an integer from 0 to 6, the —(CH₂)ₜ— moiety of the foregoing R⁵ group optionally includes a carbon-carbon double or triple bond when t is an integer from 2 to 6, and the alkyl and heterocyclic moieties of the foregoing R⁵ groups are optionally substituted by 1 to 3 substituents independently selected from —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

21. The compound of claim 17, wherein R² is a group of the formula 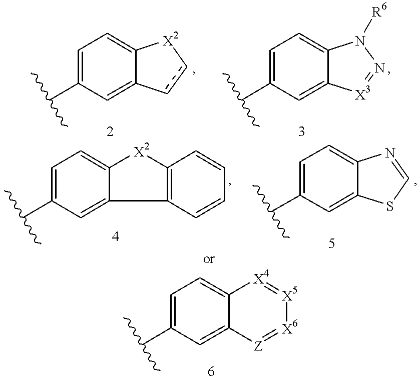
wherein X² is —S—, —N(R⁶)— or O, and X³, X⁴, X⁵, X⁶, and Z is N or CH, the dashed line in formula 2 represents an optional double bond, and the above R² groups of formulas 2, 4 and 6 are optionally substituted by 1 to 5 R⁵ substituents and the R²groups of formulas 3 and 5 are optionally substituted by 1 to 3 R⁵ substituents.

22. The compound of claim 21, wherein R²group is a group of formula 2, wherein said group is optionally substituted by 1 to 3 R⁵ substituents.

23. The compound of claim 17, wherein said compound is selected from the group consisting of:
{1-Methyl-5-[7-(2-methyl-1H-indol-5-ylamino)-thieno[3,2-b]pyridin-2-yl]-1H-imidazol-2-yl}-morpholin-4-yl-methanone;
{1-Methyl-5-[7-(2-methyl-1H-indol-5-ylamino)-thieno[3,2-b]pyridin-2-yl]-1H-imidazol-2-yl}-(4-methyl-piperazin-1-yl)-methanone;
1-Methyl-5-[7-(2-methyl-1H-indol-5-ylamino)-thieno[3,2-b]pyridin-2-yl]-1H-imidazole-2-carboxylic acid dimethylamide;
1-Methyl-5-[7-(2-methyl-1H-indol-5-ylamino)-thieno[3,2-b]pyridin-2-yl]-1H-imidazole-2-carboxylic acid methylamide;
2-{1-Methyl-5-[7-(2-methyl-1H-indol-5-ylamino)-thieno[3,2-b]pyridin-2-yl]-1H-imidazol-2-yl}-propane-1,2-diol;
1-Methyl-5-[7-(2-methyl-1H-indol-5-ylamino)-thieno[3,2-b]pyridin-2-yl]-1H-imidazole-2-carboxylic acid amide;
2-{2-[7-(2-Methyl-1H-indol-5-ylamino)-thieno[3,2-b]pyridin-2-yl]-thiazol4-yl}-propan-2-ol;
(2-methyl-1H-indol-5-yl)-(2-4 pyrrolidin-1-ylmethyl-thiazole-2-yl)-thieno[3,2-b]pyridin-7-yl)-amine; pharmaceutically acceptable salts of said compounds; solvates of said compounds; and prodrugs of said compounds.

24. A compound of the formula 1 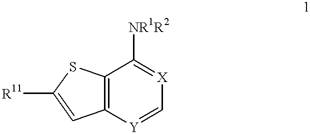
or a pharmaceutically acceptable salt, prodrug or hydrate thereof,
X is CH;
Y is N;
R¹ is H;
R² is 5 to 13 membered heterocyclic, wherein said R² group is optionally substituted by 1 to 5 R⁵ substituents,
each R⁵ is independently selected from cyano, —C(O)R⁸, —NR⁶C(O)R⁷, —C(O)NR⁶R⁷, —NR⁶R⁷, —OR⁹, —NR⁹SO₂NR⁶R⁷, —SO₂R⁶, C₁-C₆ alkyl, —(CH₂)ⱼO(CH₂)_{q}NR⁶R⁷, —(CH₂)ₜO(CH₂)_{q}OR⁹, —(CH₂)ₜOR⁹, —(CH₂)ₜ(5 to 10 membered heterocyclic), —C(O)(CH₂)ₜ(5 to 10 membered heterocyclic), —(CH₂)ⱼNR⁷(CH₂)_{q}NR⁶R⁷, —(CH₂)ⱼNR⁷CH₂C(O)NR⁶R⁷, —(CH₂)ⱼNR⁷(CH₂)_{q}NR⁹C(O)R⁸, —(CH₂)ⱼNR⁷(CH₂)ₜO(CH₂)_{q}OR⁹, and —(CH₂)ⱼNR⁷(CH₂)ₜR⁶, wherein j is an integer from 0 to 2, t is an integer from 0 to 6, q is an integer from 2 to 6, the —(CH₂)_{q}— and —(CH₂)ₜ— moieties of the foregoing R⁵ groups optionally include a carbon-carbon double or triple bond where t is an integer from 2 to 6, and the alkyl, aryl and heterocyclic moieties of the foregoing R⁵ groups are optionally substituted by 1 to 3 substituents independently selected from halo, cyano, trifluoromethyl, —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6;
each R⁶ and R⁷ is independently selected from H, C₁-C₆ alkyl, —(CH₂)ₜ(C₆-C₁₀ aryl), —(CH₂)ₜ(C₆-C₁₀ cycloalkyl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, and the alkyl, aryl and heterocyclic moieties of the foregoing R⁶ and R⁷ groups are optionally substituted by 1 to 3 substituents independently selected from halo, cyano, trifluoromethyl, —C(O)R⁸, —NR⁹C(O)R¹⁰, —C(O)NR⁹R¹⁰, —NR⁹R¹⁰, C₁-C₆ alkyl, —(CH₂)ₜ(C₆-C₁₀ aryl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, with the proviso that where R⁶ and R⁷ are both attached to the same nitrogen, then R⁶ and R⁷ are not both bonded to the nitrogen directly through an oxygen;
each R⁸ is independently selected from H, C₁-C₁₀ alkyl, —O(C₁-C₁₀ alkyl), —(CH₂)ₜ(C₆-C₁₀ aryl), and —(CH₂)ₜ(5 to 10 membered heterocyclic), wherein t is an integer from 0 to 6;
each R⁹ and R¹⁰ is independently selected from H and C₁-C₆ alkyl; and,
R¹¹ is selected from the group consisting of imidazolyl, oxazolyl, oxadiazolyl, isoxazolyl, thiazolyl and thiadiazolyl, wherein said imidazolyl, oxazolyl, oxadiazolyl, isoxazolyl, thiazolyl and thiadiazolyl are optionally substituted by 1 to 5 R⁵ groups with the proviso that compound 1 is not
[2-(3-Methyl-3H-imidazol-4-yl)-thieno[3,2-b]pyridin-7-yl]-(2-methyl-1H-indol-5-yl)-amine;
2-{1-Methyl-5-[7-(2-methyl-1H-indol-5-ylamino)-thieno[3,2-b]pyridin-2-yl]-1H-imidazol-2-yl}-propan-2-ol;
[2-(1-Methyl-1H-imidazol-2-yl)-thieno[3,2-b]pyridin-7-yl]-(2-methyl-1H-indol-5-yl)-amine;
(2-Methyl-1H-indol-5-yl)-(2-thiazol-2-yl-thieno[3,2-b]pyridin-7-yl)-amine;
2-{2-[7-(2-Methyl-1H-indol-5-ylamino)-thieno[3,2-b]pyridin-2-yl]-thiazol-5-yl}-propan-2-ol;
[2-(2-Ethoxy-thiazol-5-yl)-thieno[3,2-b]pyridin-7-yl]-(2-methyl-1H-indol-5-yl)-amine;
(2-Methyl-1H-indol-5-yl)-[2-(4-methyl-thiazol-2-yl)-thieno[3,2-b]pyridin-7-yl]-amine;
[2-(3-Methoxymethyl-3H-imidazol-4-yl)-thieno[3,2-b]pyridin-7-yl]-(2-methyl-1H-indol-5-yl)-amine;
{2-[5-(4-Methoxy-phenyl)-oxazol-2-yl]-thieno[3,2-b]pyridin-7-yl}-(2-methyl-1H-indol-5-yl)-amine; or
2-{4-Methyl-2-[7-(2-methyl-1H-indol-5-ylamino)-thieno[3,2-b]pyridin-2-yl]-thiazol-5-yl}-propan-2-ol.

25. The compound of claim 24, wherein said imidazolyl, oxazolyl and thiazolyl are optionally substituted by 1 to 5 R⁵ groups, each R⁵ is independently selected from cyano, —C(O)R⁸, —NR⁶C(O)R⁷, —C(O)NR⁶R⁷, —NR⁶R⁷, —OR⁹, C₁-C₆ alkyl, —(CH₂)ⱼO(CH₂)_{q}NR⁶R⁷, —(CH₂)ₜO(CH₂)_{q}OR⁹, —(CH₂)ₜOR⁹, —(CH₂)ₜ(5 to 10 membered heterocyclic), —C(O)(CH₂)ₜ(5 to 10 membered heterocyclic), —(CH₂)ⱼNR⁷(CH₂)_{q}NR⁶R⁷, —(CH₂)ⱼNR⁷CH₂C(O)NR⁶R⁷, —(CH₂)ⱼNR⁷(CH₂)_{q}NR⁹C(O)R⁸, —(CH₂)ⱼNR⁷(CH₂)ₜO(CH₂)_{q}OR⁹, and —(CH₂)ⱼNR⁷(CH₂)ₜR⁶, wherein j is an integer from 0 to 2, t is an integer from 0 to 6, q is an integer from 2 to 6, the —(CH₂)_{q}— and —(CH₂)ₜ— moieties of the foregoing R⁵ groups optionally include a carbon-carbon double or triple bond where t is an integer from 2 to 6, and the alkyl, aryl and heterocyclic moieties of the foregoing R⁵ groups are optionally substituted by 1 to 3 substituents independently selected from halo, cyano, trifluoromethyl, —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

26. The compound of claim 25, wherein each R⁵ is independently selected from —C(O)R⁸, —C(O)NR⁶R⁷, —NR⁶R⁷, —OR⁹, C₁-C₆ alkyl, —C(O)(CH₂)ₜ(5 to 10 membered heterocyclic), wherein t is an integer from 0 to 6, the —(CH₂)ₜ— moiety of the foregoing R⁵ group optionally includes a carbon-carbon double or triple bond when t is an integer from 2 to 6, and the alkyl and heterocyclic moieties of the foregoing R⁵ groups are optionally substituted by 1 to 3 substituents independently selected from halo, cyano, trifluoromethyl, —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

27. The compound of claim 26, wherein each R⁵ is independently selected from —C(O)R⁸, —C(O)NR⁶R⁷, C₁-C₆ alkyl, —C(O)(CH₂)ₜ(5 to 10 membered heterocyclic), wherein t is an integer from 0 to 6, the —(CH₂)ₜ— moiety of the foregoing R⁵ group optionally includes a carbon-carbon double or triple bond when t is an integer from 2 to 6, and the alkyl and heterocyclic moieties of the foregoing R⁵ groups are optionally substituted by 1 to 3 substituents independently selected from —C(O)R⁸, —NR⁶C(O)R⁷, —C(O)NR⁶R⁷, —(CH₂)ₜNR⁶R⁷ , C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

28. The compound of claim 24, wherein R² is a group of the formula 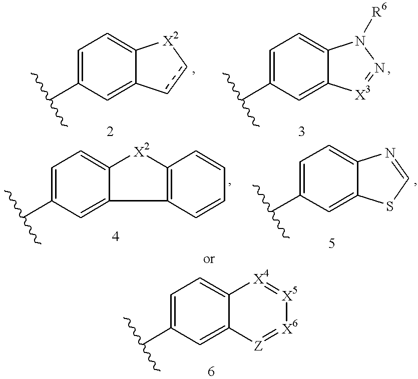
wherein X² is —S—, —N(R⁶)— or O, and X³, X⁴, X⁵, X⁶, and Z is N or CH, the dashed line in formula 2 represents an optional double bond, and the above R² groups of formulas 2, 4 and 6 are optionally substituted by 1 to 5 R⁵ substituents and the R² groups of formulas 3 and 5 are optionally substituted by 1 to 3 R⁵ substituents.

29. The compound of claim 28, wherein R² group is a group of formula 2, wherein said group is optionally substituted by 1 to 3 R⁵ substituents.

30. The compound of claim 24, wherein said compound is selected from the group consisting of:
{1-Methyl-5-[7-(2-methyl-1H-indol-5-ylamino)-thieno[3,2-b]pyridin-2-yl]-1H-imidazol-2-yl}-morpholin-4-yl-methanone;
{1-Methyl-5-[7-(2-methyl-1H-indol-5-ylamino)-thieno[3,2-b]pyridin-2-yl]-1H-imidazol-2-yl}-(4-methyl-piperazin-1-yl)-methanone;
1-Methyl-5-[7-(2-methyl-1H-indol-5-ylamino)-thieno[3,2-b]pyridin-2-yl]-1H-imidazole-2-carboxylic acid dimethylamide;
1-Methyl-5-[7-(2-methyl-1H-indol-5-ylamino)-thieno[3,2-b]pyridin-2-yl]-1H-imidazole-2-carboxylic acid methylamide;
2-{1-Methyl-5-[7-(2-methyl-1H-indol-5-ylamino)-thieno[3,2-b]pyridin-2-yl]-1H-imidazol-2-yl}-propane-1,2-diol;
1-Methyl-5-[7-(2-methyl-1H-indol-5-ylamino)-thieno[3,2-b]pyridin-2-yl]-1H-imidazole-2-carboxylic acid amide;
2-{2-[7-(2-Methyl-1H-indol-5-ylamino)-thieno[3,2-b]pyridin-2-yl]-thiazol-4-yl}-propan-2-ol;
(2-methyl-1H-indol-5-yl)-(2-4 pyrrolidin-1-ylmethyl-thiazole-2-yl)-thieno[3,2-b]pyridin-7-yl)-amine; pharmaceutically acceptable salts of said compounds; solvates of said compounds; and prodrugs of said compounds.

31. A compound of claim 1, having the formula 1 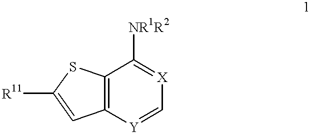
or a pharmaceutically acceptable salt, prodrug or hydrate thereof,
X is CH;
Y is N;
R¹ is H;
R² is 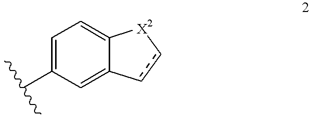
X² is —N(R⁶)—, the dashed line in formula 2 represents an optional double bond, and the above R² group of formula 2 is optionally substituted by 1 to 5 R⁵ substituents;
each R⁵ is independently selected from —C(O)R⁸, —C(O)NR⁶R⁷, —NR⁶R⁷, —OR⁹, C₁-C₆ alkyl, —C(O)(CH₂)ₜ(5 to 10 membered heterocyclic), wherein t is an integer from 0 to 6, the —(CH₂)ₜ— moiety of the foregoing R⁵ group optionally includes a carbon-carbon double or triple bond when t is an integer from 2 to 6, and the alkyl and heterocyclic moieties of the foregoing cyano, trifluoromethyl, —C(O)R⁸, —NR⁶C(O)R⁷, —C(O)NR⁶R⁷, —(CH₂)ₜNR⁶R⁷, C₁-C₆ alkyl, —CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6;
each R⁶ and R⁷ is independently selected from H, C₁-C₆ alkyl, —(CH₂)ₜ(C₆-C₁₀ aryl), —(CH₂)ₜ(C₆-C₁₀ cycloalkyl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, and the alkyl, aryl and heterocyclic moieties of the foregoing R⁶ and R⁷ groups are optionally substituted by 1 to 3 substituents independently selected from halo, cyano, trifluoromethyl, —C(O)R⁸, —NR⁹C(O)R¹⁰, —C(O)NR⁹R¹⁰, —NR⁹R¹⁰, C₁-C₆ alkyl, —(CH₂)ₜ(C₆-C₁₀ aryl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, with the proviso that where R⁶ and R⁷ are both attached to the same nitrogen, then R⁶ and R⁷ are not both bonded to the nitrogen directly through an oxygen;
each R⁸ is independently selected from H, C₁-C₁₀ alkyl, —O(C₁-C₁₀ alkyl), —(CH₂)ₜ(C₆-C₁₀ aryl), and —(CH₂)ₜ(5 to 10 membered heterocyclic), wherein t is an integer from 0 to 6;
each R⁹ and R¹⁰ is independently selected from H and C₁-C₆ alkyl; and,
R¹¹ is selected from the group consisting of imidazolyl, oxazolyl, or thiazolyl, wherein said imidazolyl, oxazolyl, or thiazolyl are optionally substituted by 1 to 5 R⁵ groups with the proviso that compound 1 is not
[2-(3-Methyl-3H-imidazol-4-yl)-thieno[3,2-b]pyridin-7-yl]-(2-methyl-1H-indol-5-yl)-amine;
2-{1-Methyl-5-[7-(2-methyl-1H-indol-5-ylamino)-thieno[3,2-b]pyridin-2-yl]-1H-imidazol-2-yl}-propan-2-ol;
[2-(1-Methyl-1H-imidazol-2-yl)-thieno[3,2-b]pyridin-7-yl]-(2-methyl-1H-indol-5-yl)-amine;
(2-Methyl-1H-indol-5-yl)-(2-thiazol-2-yl-thieno[3,2-b]pyridin-7-yl)-amine; or
2-{2-[7-(2-Methyl-1H-indol-5-ylamino)-thieno[3,2-b]pyridin-2-yl]-thiazol-5-yl}-propan-2-ol.

32. The compound of claim 31, wherein R¹¹ is thiazolyl and said thiazolyl is optionally substituted by 1 to 5 R⁵ groups.

33. The compound of claim 32, wherein each R⁵ is independently selected from —C(O)R⁸, —C(O)NR⁶R⁷, C₁-C₆ alkyl, —C(O)(CH₂)ₜ(5 to 10 membered heterocyclic), wherein t is an integer from 0 to 6, the —(CH₂)ₜ— moiety of the foregoing R⁵ group optionally includes a carbon-carbon double or triple bond when t is an integer from 2 to 6, and the alkyl and heterocyclic moieties of the foregoing R⁵ groups are optionally substituted by 1 to 3 substituents independently selected from —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

34. The compound of claim 31, wherein R² is 2-methyl-1H-indol-5-ylamino.

35. A compound of claim 1, having the formula 1 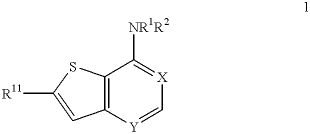
or a pharmaceutically acceptable salt, prodrug or hydrate thereof,
X is CH;
Y is N:
R¹ is H;
R² is 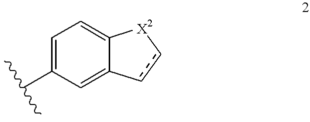
X² is —N(R⁶)—, the dashed line in formula 2 represents an optional double bond, and the above R² group of formula 2 is optionally substituted by 1 to 5 R⁵ substituents;
each R⁵ is independently selected from —C(O)R⁸, —C(O)NR⁶R⁷, —NR⁶R⁷, —OR⁹, C₁-C₆ alkyl, —C(O)(CH₂)ₜ(5 to 10 membered heterocyclic), wherein t is an integer from 0 to 6, the —(CH₂)ₜ— moiety of the foregoing R⁵ group optionally includes a carbon-carbon double or triple bond when t is an integer from 2 to 6, and the alkyl and heterocyclic moieties of the foregoing R⁵ groups are optionally substituted by 1 to 3 substituents independently selected from halo, cyano, trifluoromethyl, —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6;
each R⁶ and R⁷ is independently selected from H, C₁-C₆ alkyl, —(CH₂)ₜ(C₆-C₁₀ aryl), —(CH₂)ₜ(C₆-C₁₀ cycloalkyl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, and the alkyl, aryl and heterocyclic moieties of the foregoing R⁶ and R⁷ groups are optionally substituted by 1 to 3 substituents independently selected from halo, cyano, trifluoromethyl, —C(O)R⁸, —NR⁹C(O)R¹⁰, —C(O)NR⁹R¹⁰, —NR⁹R¹⁰, C₁-C₆ alkyl, —(CH₂)ₜ(C₆-C₁₀ aryl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, with the proviso that where R⁶ and R⁷ are both attached to the same nitrogen, then R⁶ and R⁷ are not both bonded to the nitrogen directly through an oxygen;
each R⁸ is independently selected from H, C₁-C₁₀ alkyl, —O(C₁-C₁₀ alkyl), —(CH₂)ₜ(C₆-C₁₀ aryl), and —(CH₂)ₜ(5 to 10 membered heterocyclic), wherein t is an integer from 0 to 6;
each R⁹ and R¹⁰ is independently selected from H and C₁-C₆ alkyl; and,
R¹¹ is selected from the group consisting of imidazolyl, oxazolyl, or thiazolyl, wherein said imidazolyl, oxazolyl, or thiazolyl are optionally substituted by 1 to 5 R⁵ groups with the proviso that compound 1 is not
[2-(3-Methyl-3H-imidazol-4-yl)-thieno[3,2-b]pyridin-7-yl]-(2-methyl-1H-indol-5-yl)-amine;
2-{1-Methyl-5-[7-(2-methyl-1H-indol-5-ylamino)-thieno[3,2-b]pyridin-2-yl]-1H-imidazol-2-yl}-propan-2-ol;
[2-(1-Methyl-1H-imidazol-2-yl)-thieno[3,2-b]pyridin-7-yl]-(2-methyl-1H-indol-5-yl)-amine;
(2-Methyl-1H-indol-5-yl)-(2-thiazol-2-yl-thieno[3,2-b]pyridin-7-yl)-amine;
2-{2-[7-(2-Methyl-1H-indol-5-ylamino)-thieno[3,2-b]pyridin-2-yl]-thiazol-5-yl}-propan-2-ol;
[2-(2-Ethoxy-thiazol-5-yl)-thieno[3,2-b]pyridin-7-yl]-(2-methyl-1H-indol-5-yl)-amine;
(2-Methyl-1H-indol-5-yl)-[2-(4-methyl-thiazol-2-yl)-thieno[3,2-b]pyridin-7-yl]-amine;
[2-(3-Methoxymethyl-3H-imidazol-4-yl)-thieno[3,2-b]pyridin-7-yl]-(2-methyl-1H-indol-5-yl)-amine;
{2-[5-(4-Methoxy-phenyl)-oxazol-2-yl]-thieno[3,2-b]pyridin-7-yl}-(2-methyl-1H-indol-5-yl)-amine; or
2-{4-Methyl-2-[7-(2-methyl-1H-indol-5-ylamino)-thieno[3,2-b]pyridin-2-yl]-thiazol-5-yl}-propan-2-ol.

36. The compound of claim 35, wherein R¹¹ is thiazolyl and said thiazolyl is optionally substituted by 1 to 5 R⁵ groups.

37. The compound of claim 36, wherein each R⁵ is independently selected from —C(O)R⁸, —C(O)NR⁶R⁷, C₁-C₆ alkyl, —C(O)(CH₂)ₜ(5 to 10 membered heterocyclic), wherein t is an integer from 0 to 6, the —(CH₂)ₜ— moiety of the foregoing R⁵ group optionally includes a carbon-carbon double or triple bond when t is an integer from 2 to 6, and the alkyl and heterocyclic moieties of the foregoing R⁵ groups are optionally substituted by 1 to 3 substituents independently selected from —C(O)R⁸, —NR⁶C(O)R⁷, —C(O)NR⁶R⁷, —(CH₂)ₜNR⁶R⁷, C₁-C₆ alkyl, —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6.

38. The compound of claim 39, wherein R² is 2-methyl-1H-indol-5-ylamino.

39. The compound of claim 1, having the formula 1 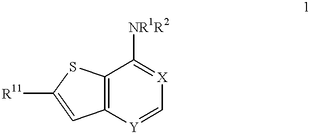
or a pharmaceutically acceptable salt, prod rug or hydrate thereof,
X is CH;
Y is N;
R¹ is H;
R² is 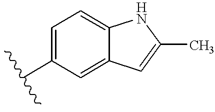
wherein each R⁵ is independently selected from —C(O)R⁸, —C(O)NR⁶R⁷, C₁-C₆ alkyl, —C(O)(CH₂)ₜ(5 to 10 membered heterocyclic), wherein t is an integer from 0 to 6;
each R⁶ and R⁷ is independently selected from H, C₁-C₆ alkyl, —(CH₂)ₜ(C₆-C₁₀ aryl), —(CH₂)ₜ(C₆-C₁₀ cycloalkyl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, and the alkyl, aryl and heterocyclic moieties of the foregoing R⁶ and R⁷ groups are optionally substituted by 1 to 3 substituents independently selected from halo, cyano, trifluoromethyl, —C(O)R⁸, —NR⁹C(O)R¹⁰, —C(O)NR⁹R¹⁰, —NR⁹R¹⁰, C₁-C₆ alkyl, —(CH₂)ₜ(C₆-C₁₀ aryl), —(CH₂)ₜ(5 to 10 membered heterocyclic), —(CH₂)ₜO(CH₂)_{q}OR⁹, and —(CH₂)ₜOR⁹, wherein t is an integer from 0 to 6 and q is an integer from 2 to 6, with the proviso that where R⁶ and R⁷ are both attached to the same nitrogen, then R⁶ and R⁷ are not both bonded to the nitrogen directly through an oxygen;
each R⁸ is independently selected from H, C₁-C₁₀ alkyl, —O(C₁-C₁₀ alkyl), —(CH₂)ₜ(C₆-C₁₀ aryl), and —(CH₂)ₜ(5 to 10 membered heterocyclic), wherein t is an integer from 0 to 6;
each R⁹ and R¹⁰ is independently selected from H and C₁-C₆ alkyl; and,
R¹¹ is selected from the group consisting of imidazolyl, oxazolyl, or thiazolyl, wherein said imidazolyl, oxazolyl, or thiazolyl are optionally substituted by 1 to 5 R⁵ groups with the proviso that compound 1 is not
[2-(3-Methyl-3H-imidazol-4-yl)-thieno[3,2-b]pyridin-7-yl]-(2-methyl-1H-indol-5-yl)-amine;
2-{1-Methyl-5-[7-(2-methyl-1H-indol-5-ylamino)-thieno[3,2-b]pyridin-2-yl]-1H-imidazol-2-yl}-propan-2-ol;
[2-(1-Methyl-1H-imidazol-2-yl)-thieno[3,2-b]pyridin-7-yl]-(2-methyl-1H-indol-5-yl)-amine;
(2-Methyl-1H-indol-5-yl)-(2-thiazol-2-yl-thieno[3,2-b]pyridin-7-yl)-amine; or
2-{2-[7-(2-Methyl-1H-indol-5-ylamino)-thieno[3,2-b]pyridin-2-yl]-thiazol-5-yl}-propan-2-ol.

40. The compound of claim 39, wherein each R⁶ and R⁷ is independently selected from H, C₁-C₆ alkyl, —(CH₂)ₜ(5 to 10 membered heterocyclic), and —(CH₂)ₜOR⁹, wherein t is an integer from 0 to 6 with the proviso that where R⁶ and R⁷ are both attached to the same nitrogen, then R⁶ and R⁷ are not both bonded to the nitrogen directly through an oxygen.

41. The compound of claim 40, wherein R¹¹ is thiazolyl and wherein said thiazolyl is optionally substituted by 1 to 5 R⁵ groups.

42. A compound of claim 1, having the formula 1 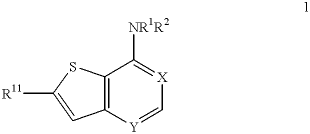
or a pharmaceutically acceptable salt, prodrug or hydrate thereof,
X is CH;
Y is N;
R¹ is H;
R² is 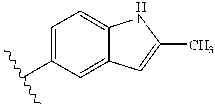
wherein each R⁵ is independently selected from —C(O)R⁸, —C(O)NR⁶R⁷, C₁-C₆ alkyl, —C(O)(CH₂)ₜ(5 to 10 membered heterocyclic), wherein t is an integer from 0 to 6;
wherein each R⁶ and R⁷ is independently selected from H, C₁-C₆ alkyl, —(CH₂)ₜ(5 to 10 membered heterocyclic), and —(CH₂)ₜOR⁹, wherein t is an integer from 0 to 6, with the proviso that where R⁶ and R⁷ are both attached to the same nitrogen, then R⁶ and R⁷ are not both bonded to the nitrogen directly through an oxygen;
each R⁸ is independently selected from H, C₁-C₁₀ alkyl, —(CH₂)ₜ(C₆-C₁₀ aryl), and —(CH₂)ₜ(5 to 10 membered heterocyclic), wherein t is an integer from 0 to 6;
each R⁹ and R¹⁰ is independently selected from H and C₁-C₆ alkyl; and,
R¹¹ is thiazolyl wherein said thiazolyl is optionally substituted by 1 to 5 R⁵ groups with the proviso that compound 1 is not
(2-Methyl-1H-indol-5-yl)-(2-thiazol-2-yl-thieno[3,2-b]pyridin-7-yl)-amine; or
2-{2-[7-(2-Methyl-1H-indol-5-ylamino)-thieno[3,2-b]pyridin-2-yl]-thiazol-5-yl}-propan-2-ol.

43. The compound of claim 1, having the formula 1 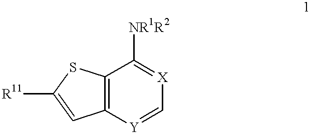
or a pharmaceutically acceptable salt, prodrug or hydrate thereof,
X is CH;
Y is N;
R¹ is H;
R² is 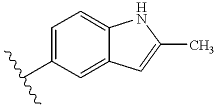
wherein each R⁵ is independently selected from —C(O)R⁸, —C(O)NR⁶R⁷, C₁-C₆ alkyl —C(O)(CH₂)ₜ(5 to 10 membered heterocyclic), wherein t is an integer from 0 to 6;
wherein each R⁶ and R⁷ is independently selected from H, C₁-C₁₀ alkyl, —(CH₂)ₜ(5 to 10 membered heterocyclic), and —(CH₂)ₜOR⁹, wherein t is an integer from 0 to 6, with the proviso that where R⁶ and R⁷ are both attached to the same nitrogen, then R⁶ and R⁷ are not both bonded to the nitrogen directly through an oxygen;
each R⁸ is independently selected from H, C₁-C₁₀ alkyl, —(CH₂)ₜ(C₆-C₁₀ aryl), and —(CH₂)ₜ(5 to 10 membered heterocyclic), wherein t is an integer from 0 to 6;
each R⁹ and R¹⁰ is independently selected from H and C₁-C₆ alkyl; and,
R¹¹ is oxazolyl, wherein said oxazolyl is optionally substituted by 1 to 5 R⁵ groups.

44. A compound of claim 1, having the formula 1 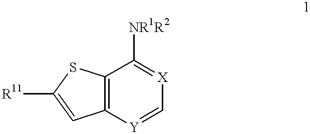
or a pharmaceutically acceptable salt, prodrug or hydrate thereof,
X is CH;
Y is N;
R¹ is H;
R² is 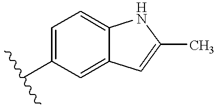
wherein each R⁵ is independently selected from —C(O)R⁸, —C(O)NR⁶R⁷, C₁-C₆ alkyl, —C(O)(CH₂)ₜ(5 to 10 membered heterocyclic), wherein t is an integer from 0 to 6;
wherein each R⁶ and R⁷ is independently selected from H, C₁-C₆ alkyl, —(CH₂)ₜ(5 to 10 membered heterocyclic), and —(CH₂)ₜOR⁹, wherein t is an integer from 0 to 6, with the proviso that where R⁶ and R⁷ are both attached to the same nitrogen, then R⁶ and R⁷ are not both bonded to the nitrogen directly through an oxygen;
each R⁸ is independently selected from H, C₁-C₁₀ alkyl, —(CH₂)ₜ(C₆-C₁₀ aryl), and —(CH₂)ₜ(5 to 10 membered heterocyclic), wherein t is an integer from 0 to 6;
each R⁹ and R¹⁰ is independently selected from H and C₁-C₆ alkyl; and,
R¹¹ is imidazolyl wherein said imidazolyl is optionally substituted by 1 to 5 R⁵ groups with the proviso that compound 1 is not
[2-(3-Methyl-3H-imidazol-4-yl)-thieno[3,2-b]pyridin-7-yl]-(2-methyl-1H-indol-5-yl)-amine;
2-{1-Methyl-5-[7-(2-methyl-1H-indol-5-ylamino)-thieno[3,2-b]pyridin-2-yl]-1H-imidazol-2-yl}-propan-2-ol; or
[2-(1-Methyl-1H-imidazol-2-yl)-thieno[3,2-b]pyridin-7-yl]-(2-methyl-1H-indol-5-yl)-amine.

45. A pharmaceutical composition for the treatment of a hyperproliferative disorder in a mammal which comprises a therapeutically effective amount of a compound of claim 1 and a pharmaceutically acceptable carrier.

46. The pharmaceutical composition of claim 45, wherein said hyperproliferative disorder is cancer.

47. The pharmaceutical composition of claim 46, wherein said cancer is brain, lung, kidney, renal, ovarian, squamous cell, bladder, gastric, pancreatic, breast, head, neck, oesophageal, gynecological, prostate, colorectal or thyroid cancer.

48. The pharmaceutical composition of claim 45, wherein said hyperproliferative disorder is noncancerous.

49. The pharmaceutical composition of claim 48, wherein said disorder is a benign hyperplasia of the skin or prostate.

50. A pharmaceutical composition for the treatment of a hyperproliferative disorder in a mammal which comprises a therapeutically effective amount of a compound of claim 1 in combination with an anti-tumor agent selected from the group consisting of mitotic inhibitors, alkylating agents, anti-metabolites, intercalating antibiotics, enzymes, topoisomerase inhibitors, biological response modifiers, anti-hormones, and anti-androgens, and a pharmaceutically acceptable carrier.

51. A pharmaceutical composition for the treatment of pancreatitis or kidney disease in a mammal which comprises a therapeutically effective amount of a compound of claim 1 and a pharmaceutically acceptable carrier.

52. A pharmaceutical composition for the blastocyte implantation in a mammal which comprises a therapeutically effective amount of a compound of claim 1 and a pharmaceutically acceptable carrier.

53. A pharmaceutical composition for treating a disease related to vasculogenesis or angiogenesis in a mammal which comprises a therapeutically effective amount of a compound of claim 1 and a pharmaceutically acceptable carrier.

54. The pharmaceutical composition of claim 53 wherein said disease is selected from the group consisting of tumor angiogenesis, chronic inflammatory disease such as rheumatoid arthritis, atherosclerosis, skin diseases such as psoriasis, excema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

55. A method of treating a hyperproliferative disorder in a mammal which comprises administering to said mammal a therapeutically effective amount of a compound of claim 1.

56. The method of claim 55 wherein said hyperproliferative disorder is cancer.

57. The method of claim 56 wherein said cancer is brain, lung, squamous cell, renal, kidney, ovarian, bladder, gastric, pancreatic, breast, head, neck, oesophageal, prostate, colorectal, gynecological or thyroid cancer.

58. The method of claim 55 wherein said hyperproliferative disorder is noncancerous.

59. The method of claim 58 wherein said disorder is a benign hyperplasia of the skin or prostate.

60. A method for the treatment of a hyperproliferative disorder in a mammal which comprises administering to said mammal a therapeutically effective amount of a compound of claim 1 in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

61. A method of treating pancreatitis or kidney disease in a mammal which comprises administering to said mammal a therapeutically effective amount of a compound of claim 1.

62. A method of preventing blastocyte implantation in a mammal which comprises administering to said mammal a therapeutically effective amount of a compound of claim 1.

63. A method for treating a disease related to vasculogenesis or angiogenesis in a mammal which comprises administering to said mammal a therapeutically effective amount of a compound of claim 1.

64. The method of claim 63, wherein said disease is selected from the group consisting of tumor angiogenesis, chronic inflammatory disease such as rheumatoid arthritis, atherosclerosis, skin diseases such as psoriasis, excema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.
